(19) United States
(12) Patent Application Publication
(10) Pub. No.: US 2009/0062218 A1
(43) Pub. Date: Mar. 5, 2009

(54) MACROLONE COMPOUNDS

(75) Inventors: Andrew Keith Forrest, Essex (GB); Robert John Sheppard, Essex (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(73) Assignee: GLAXO GROUP LIMITED, Greenford (GB)

(21) Appl. No.: 11/718,532

(22) PCT Filed: Nov. 9, 2005
(86) PCT No.: PCT/EP05/12036

§ 371 Date: May 3, 2007

(30) Foreign Application Priority Data
Nov. 11, 2004 (GB) 0424951.2

(51) Int. Cl. A61K 31/7048; C07H 17/08; A61P 31/00

(52) U.S. Cl. 514/29; 536/7.3; 514/28

(57) ABSTRACT

A compound of formula (I)

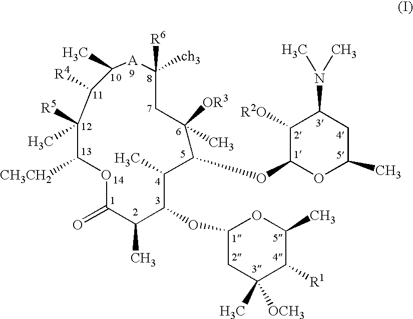

compositions comprising same, processes for their preparation and use of said compounds, particularly in the treatment of microbial infections.

The present invention relates to novel semi-synthetic macrolides having antimicrobial activity, in particular antibacterial activity. More particularly, the invention relates to 14- and 15-membered macrolides substituted at the 4″ position, to processes for their preparation, to compositions containing them and to their use in medicine.

Macrolide antibacterial agents are known to be useful in the treatment or prevention of bacterial infections. However, the emergence of macrolide-resistant bacterial strains has resulted in the need to develop new macrolide compounds.

According to the present invention, we have now found novel 14- and 15-membered macrolides substituted at the 4″ position which also have antimicrobial activity.

Thus, the present invention provides compounds of general formula (I)

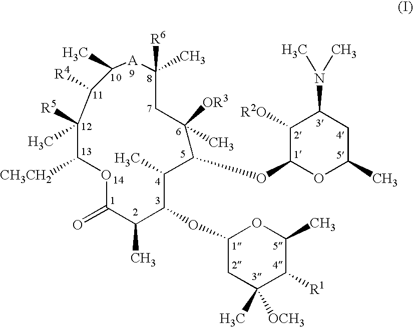

wherein
A is a bivalent radical —C(O)—, —N(R⁷)—CH₂—, —CH(NR⁸R⁹)— or —C(═NR¹⁰)—, or A and R⁴ taken together with the intervening atoms form a cyclic group having the following formula:

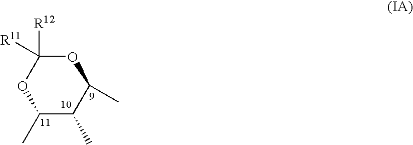

### R¹ is —OS(O)₂(CH₂)₂U¹R¹³,

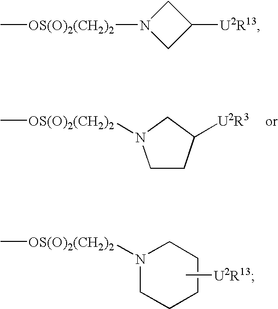

R² is hydrogen or a hydroxyl protecting group;
R³ is hydrogen, C₁₋₄alkyl, or C₃₋₆alkenyl optionally substituted by 9- or 10-membered fused bicyclic heteroaryl;
R⁴ is hydroxy, C₃₋₆alkenyloxy optionally substituted by 9- or 10-membered fused bicyclic heteroaryl, or C₁₋₆alkoxy optionally substituted by C₁₋₆alkoxy or —O(CH₂)_{d}NR⁷R¹⁴, or R⁴ and
A taken together with the intervening atoms form a cyclic group of formula (IA),
R⁵ is hydroxy, or
R⁴ and R⁵ taken together with the intervening atoms form a cyclic group having the following formula:

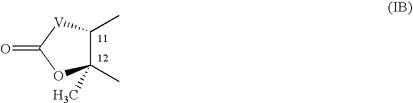

wherein V is a bivalent radical —CH₂—, —CH(CN)—, —O—, —N(R¹⁵)— or —CH(SR¹⁵)—;
R⁶ is hydrogen or fluorine;
R⁷ is hydrogen or C₁₋₆alkyl;
R⁸ and R⁹ are each independently hydrogen, C₁₋₆alkyl or —C(O)R¹⁶, or
R⁸ and R⁹ together form ═CH(CR¹⁶R¹⁷)ₑaryl, ═CH(CR¹⁶R¹⁷)ₐheterocyclyl, ═CR¹⁶R¹⁷ or ═C(R¹⁶)C(O)OR¹⁶, wherein the alkyl, aryl and heterocyclyl groups are optionally substituted by up to three groups independently selected from R¹⁸;

### R¹⁰ is —OR¹⁹;

R¹¹ and R¹² are each independently hydrogen, C₁₋₆alkyl, heteroaryl, or aryl optionally substituted by one or two groups independently selected from hydroxyl and C₁₋₆alkoxy;
R¹³ is a heterocyclic group having one of the following formulae:

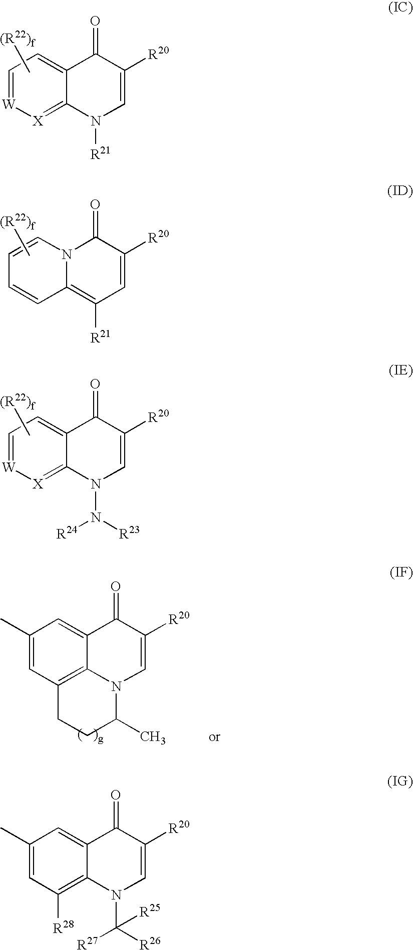

R¹⁴, R¹⁶ and R¹⁷ are each independently hydrogen or C₁₋₆alkyl;
R¹⁵ is hydrogen or C₁₋₄alkyl optionally substituted by a group selected from optionally substituted phenyl, optionally substituted 5- or 6-membered heteroaryl and optionally substituted 9- or 10-membered fused bicyclic heteroaryl;
R¹⁸ is halogen, cyano, nitro, trifluoromethyl, azido, —C(O)R²⁹, —C(O)OR²⁹, —OC(O)R²⁹, —OC(O)OR², —NR³⁰C(O)R³¹, —C(O)NR³⁰R³¹, —NR³OR³¹, hydroxy, C₁₋₆alkyl, —S(O)ₕC₁₋₆alkyl, C₁₋₆alkoxy, —(CH₂)ᵢaryl or —(CH₂)ⱼheteroaryl, wherein the alkoxy group is optionally substituted by up to three groups independently selected from —NR¹⁶R¹⁷, halogen and —OR¹⁶, and the aryl and heteroaryl groups are optionally substituted by up to five groups independently selected from halogen, cyano, nitro, trifluoromethyl, azido, —C(O)R³², —C(O)OR³², —OC(O)OR³², —NR³⁴C(O)R³⁴, —C(O)NR³³R³⁴, —NR³³R³⁴, hydroxy, C₁₋₆alkyl and C₁₋₆alkoxy;
R¹⁹ is hydrogen, C₁₋₆alkyl, C₃₋₇cycloalkyl, C₃₋₆alkenyl or a 5- or 6-membered heterocyclic group, wherein the alkyl, cycloalkyl, alkenyl and heterocyclic groups are optionally substituted by up to three groups independently selected from optionally substituted 5- or 6-membered heterocyclic group, optionally substituted 5- or 6-membered heteroaryl, —OR³⁵, —S(O)ⱼR³⁵, —NR³⁵R³⁶, —CONR³⁵R³⁶, halogen and cyano;

### R²⁰ is —C(O)OR³⁷, —C(O)NHR³⁷, —C(O)CH₂NO₂ or —C(O)CH₂SO₂R⁷;

R²¹ is hydrogen,
C₁₋₄alkyl optionally substituted by up to three groups independently selected from hydroxyl, C₁₋₄alkoxy and halogen,
C₁₋₄alkoxy,
C₂₋₆alkenyl,
C₃₋₇cycloalkyl, or
optionally substituted phenyl or benzyl, or
   R²¹ and R⁴⁴ are linked to form the bivalent radical —(CH₂)ₖ—;
   R²² is halogen, C₁₋₄alkyl, C₁₋₄thioalkyl, C₁₋₄alkoxy, —NH₂, —NH(C₁₋₄alkyl) or —N(C₁₋₄alkyl)₂;
   R²³ and R²⁴ are each independently hydrogen, C₁₋₄alkyl or C₃₋₇cycloalkyl, wherein the alkyl and cycloalkyl groups are optionally substituted by up to three groups independently selected from hydroxy, cyano, C₁₋₄alkoxy, —CONR³⁸R³⁹ and —NR³⁸R³⁹, R²³ and R²⁴, together with the nitrogen atom to which they are bound, form a 5- or 6-membered heterocyclic ring optionally containing one additional heteroatom selected from oxygen, sulfur and N—R⁴⁰, or
   R²³ is C₁₋₄alkyl, X is —C(R⁴⁴)—, and R²⁴ and R⁴⁴ are linked to form a cyclic group having the following formula:

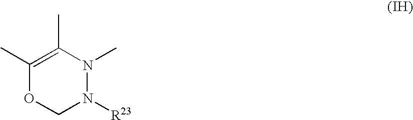

R²⁵ and R²⁶ are each independently hydrogen or methyl;
R²⁷ and R²⁸ are linked to form a bivalent radical —OCH₂—, —CH₂O—, —O(CH₂)₂—, —CH₂OCH₂— or —(CH₂)₂O—;
R²⁹ is hydrogen, C₁₋₁₀alkyl, —(CH₂)ₘaryl or —(CH₂)ₘheteroaryl;
R³⁰ and R³¹ are each independently hydrogen, —OR¹⁶, C₁₋₄alkyl, —(CH₂)ₙaryl or —(CH₂)ₙheterocyclyl;
R³² is hydrogen, C₁₋₁₀alkyl, —(CH₂)ₚaryl or —(CH₂)ₚheteroaryl;
R³³ and R³⁴ are each independently hydrogen, —OR¹⁶, C₁₋₆alkyl, —(CH₂)_{q}aryl or —(CH₂)_{q}heterocyclyl;
R³⁵ and R³⁸ are each independently hydrogen, C₁₋₄alkyl or C₁₋₄alkoxyC₁₋₄alkyl;
R³⁷ is hydrogen,
C₁₋₆alkyl optionally substituted by up to three groups independently selected from halogen, cyano, C₁₋₄alkoxy optionally substituted by phenyl or C₁₋₄alkoxy, —C(O)C₁₋₆alkyl, —C(O)OC₁₋₆alkyl, —OC(O)C₁₋₆alkyl, —OC(O)OC₁₋₆alkyl, —C(O)NR⁴¹R⁴², —NR⁴¹R⁴² and phenyl optionally substituted by nitro or —C(O)OC₁₋₆alkyl,
—(CH₂)ᵣC₃₋₇cycloalkyl,
—(CH₂)ᵣheterocyclyl,
—(CH₂)ᵣheteroaryl,
—(CH₂)ᵣaryl,
C₃₋₆alkenyl, or
C₃₋₆alkynyl;
   R³⁸ and R³⁹ are each independently hydrogen or C₁₋₄alkyl;
   R⁴⁰ is hydrogen or methyl;
   R⁴¹ and R⁴² are each independently hydrogen or C₁₋₆alkyl optionally substituted by phenyl or —C(O)OC₁₋₆alkyl, or
   R⁴¹ and R⁴², together with the nitrogen atom to which they are bound, form a 5- or 6-membered heterocyclic group optionally containing one additional heteroatom selected from oxygen, sulfur and N—R⁴⁰;
   R⁴³ is hydrogen, C₁₋₄alkyl, C₃₋₇cycloalkyl, optionally substituted phenyl or benzyl, acetyl or benzoyl;
   R⁴⁴ is hydrogen or R²², or, when X is —C(R⁴⁴)—, R⁴⁴ and R²⁴ may be linked to form a cyclic group of formula (IH) or R⁴⁴ and R²¹ may be linked to form the bivalent radical —(CH₂)ₖ—;
   U¹ is a bivalent radical —Y(CH₂)₅B—, —Y(CH₂)ₛ—, —Y(CH₂)₃B(CH₂)ₜD-, —Y(CH₂)ₛB(CH₂)ₜ—, —Y(CH₂)ₛB(CH₂)ₜD(CH₂)ᵤE- or —Y(CH₂)ₛB(CH₂)ₜD(CH₂)ᵤ—;
   U² is U¹ or a bivalent radical —O—, —N(R⁴³)—, —S(O)ᵥ— or —CH₂—;
   Y, B, D and E are each independently a bivalent radical —N(R⁴³)—, —O—, —S(O)ᵥ—, —N(R⁴³)C(O)—, —C(O)N(R⁴³)— or —N[C(O)R⁴³]—,
   W and X are each independently —C(R⁴⁴)— or a nitrogen, with the proviso that W and X are not both nitrogen;
   d is an integer from 2 to 4;
   e, i, m, n, p, q and r are each independently integers from 0 to 4;
   f, h, j and v are each independently integers from 0 to 2;
   g is 0 or 1;
   s, t and u are each independently integers from 2 to 5;
   k is 2 or 3;
   and pharmaceutically acceptable derivatives thereof.

The term “pharmaceutically acceptable” as used herein means a compound which is suitable for pharmaceutical use. Salts and solvates of compounds of the invention, which are suitable for use in medicine are those wherein the counterion or associated solvent is pharmaceutically acceptable. However, salts and solvates having non-pharmaceutically acceptable counterions or associated solvents are within the scope of the present invention, for example, for use as intermediates in the preparation of other compounds of the invention and their pharmaceutically acceptable salts and solvates.

The term “pharmaceutically acceptable derivative” as used herein means any pharmaceutically acceptable salt, solvate or prodrug, e.g. ester, of a compound of the invention, which upon administration to the recipient is capable of providing (directly or indirectly) a compound of the invention, or an active metabolite or residue thereof. Such derivatives are recognizable to those skilled in the art, without undue experimentation. Nevertheless, reference is made to the teaching of Burger's Medicinal Chemistry and Drug Discovery, 5^{th} Edition, Vol 1: Principles and Practice, which is incorporated herein by reference to the extent of teaching such derivatives. Examples of pharmaceutically acceptable derivatives are salts, solvates, esters, carbamates and phosphate esters. Additional examples of pharmaceutically acceptable derivatives are salts, solvates and esters. Further examples of pharmaceutically acceptable derivatives are salts and esters, such as salts.

The compounds of the present invention may be in the form of and/or may be administered as a pharmaceutically acceptable salt. For a review on suitable salts see Berge et al., J. Pharm. Sci., 1977, 66, 1-19.

Typically, a pharmaceutical acceptable salt may be readily prepared by using a desired acid or base as appropriate. The salt may precipitate from solution and be collected by filtration or may be recovered by evaporation of the solvent. For example, an aqueous solution of an acid such as lactobionic acid may be added to a solution of a compound of formula (I) in a solvent such as acetonitrile, acetone or THF, and the resulting mixture evaporated to dryness, redissolved in water and lyophilised to obtain the acid addition salt as a solid. Alternatively, a compound of formula (I) may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

The skilled person will appreciate that where the compound of formula (I) contains more than one basic group bis salts (2:1 acid:compound of formula (I)) or tris salts (3:1 acid:compound of formula (I)) may also be formed and are salts according to the present invention.

Suitable addition salts are formed from inorganic or organic acids which form non-toxic salts and examples are lactobionate, mandelate (including (S)-(+)-mandelate, (R)-(−)-mandelate and (R,S)-mandelate), hydrochloride, hydrobromide, hydroiodide, sulfate, bisulfate, nitrate, phosphate, hydrogen phosphate, acetate, trifluoroacetate, maleate, malate, fumarate, lactate, tartrate, citrate, formate, gluconate, succinate, ethyl succinate (4-ethoxy-4-oxo-butanoate), pyruvate, oxalate, oxaloacetate, saccharate, benzoate, alkyl or aryl sulphonates (eg methanesulphonate, ethanesulphonate, benzenesulphonate or p-toluenesulphonate) and isethionate. In one embodiment, suitable salts include lactobionate, citrate, succinate, (L)-(+)-tartrate, (S)-(+)-mandalete and bis-(S)-(+)-mandalete, for example lactobionate, citrate, succinate and (L)-(+)-tartrate, such as lactobionate and citrate.

Pharmaceutically acceptable base salts include ammonium salts, alkali metal salts such as those of sodium and potassium, alkaline earth metal salts such as those of calcium and magnesium and salts with organic bases, including salts of primary, secondary and tertiary amines, such as isopropylamine, diethylamine, ethanolamine, trimethylamine, dicyclohexyl amine and N-methyl-D-glucamine.

Compounds of the invention may have both a basic and an acidic centre may therefore be in the form of zwitterions.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. Solvates of the compounds of the invention are within the scope of the invention. The salts of the compound of formula (I) may form solvates (e.g. hydrates) and the invention also includes all such solvates.

The term “prodrug” as used herein means a compound which is converted within the body, e.g. by hydrolysis in the blood, into its active form that has medical effects. Pharmaceutically acceptable prodrugs are described in T. Higuchi and V. Stella, “prodrugs as Novel Delivery Systems”, Vol. 14 of the A.C.S. Symposium Series; Edward B. Roche, ed., “Bioreversible Carriers in Drug Design”, American Pharmaceutical Association and Pergamon Press, 1987; and in D. Fleisher, S. Ramon and H. Barbra “Improved oral drug delivery: solubility limitations overcome by the use of prodrugs”, Advanced Drug Delivery Reviews (1996) 19(2) 115-130, each of which are incorporated herein by reference.

Prodrugs are any covalently bonded carriers that release a compound of formula (I) in vivo when such prodrug is administered to a patient. Prodrugs are generally prepared by modifying functional groups in a way such that the modification is cleaved, either by routine manipulation or in vivo, yielding the parent compound. Prodrugs include, for example, compounds of this invention wherein hydroxy, amine or sulfhydryl groups are bonded to any group that, when administered to a patient, cleaves to form the hydroxy, amine or sulfhydryl groups. Thus, representative examples of prodrugs include (but are not limited to) acetate, formate and benzoate derivatives of alcohol, sulfhydryl and amine functional groups of the compounds of formula (I). Further, in the case of a carboxylic acid (—COOH), esters may be employed, such as methyl esters, ethyl esters, and the like. Esters may be active in their own right and/or be hydrolysable under in vivo conditions in the human body. Suitable pharmaceutically acceptable in vivo hydrolysable ester groups include those which break down readily in the human body to leave the parent acid or its salt.

References hereinafter to a compound according to the invention include both compounds of formula (I) and their pharmaceutically acceptable derivatives.

With regard to stereoisomers, the compounds of formula (I) have more than one asymmetric carbon atom. In the general formula (I) as drawn, the solid wedge shaped bond indicates that the bond is above the plane of the paper. The broken bond indicates that the bond is below the plane of the paper.

It will be appreciated that the substituents on the macrolide may also have one or more asymmetric carbon atoms. Thus, the compounds of structure (I) may occur as individual enantiomers or diastereomers. All such isomeric forms are included within the present invention, including mixtures thereof.

Where a compound of the invention contains an alkenyl group, cis (Z) and trans (E) isomerism may also occur. The present invention includes the individual stereoisomers of the compound of the invention and, where appropriate, the individual tautomeric forms thereof, together with mixtures thereof.

Separation of diastereoisomers or cis and trans isomers may be achieved by conventional techniques, e.g. by fractional crystallisation, chromatography or HPLC. A stereoisomeric mixture of the agent may also be prepared from a corresponding optically pure intermediate or by resolution, such as by HPLC, of the corresponding mixture using a suitable chiral support or by fractional crystallisation of the diastereoisomeric salts formed by reaction of the corresponding mixture with a suitable optically active acid or base, as appropriate.

The compounds of formula (I) may be in crystalline or amorphous form. Furthermore, some of the crystalline forms of the compounds of structure (I) may exist as polymorphs, which are included in the present invention.

Compounds wherein R² represents a hydroxyl protecting group are in general intermediates for the preparation of other compounds of formula (I).

When the group OR² is a protected hydroxyl group this is conveniently an ether or an acyloxy group. Examples of particularly suitable ether groups include those in which R² is a trialkylsilyl (i.e. trimethylsilyl). When the group OR² represents an acyloxy group, then examples of suitable groups R² include acetyl or benzoyl.

When R¹is

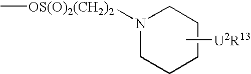

the —U²R¹³ group is typically attached at the 3- or 4-position on the piperidine ring.

When R¹³ is a heterocyclic group having the following structure:

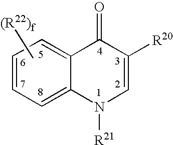

said heterocyclic is linked in the 5, 6, 7 or 8 position to the U¹ or U² group as above defined. In one embodiment, the heterocyclic is linked in the 6 or 7 position, for example the 6 position. In another embodiment, the heterocyclic is linked in the 5 or 8 position. When present, the R²² group or groups may be attached at any otherwise vacant or unoccupied position on the ring, for example an R²² group may be attached at the 7 position.

When R¹³ is a heterocyclic group having the following structure:

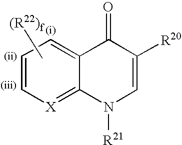

wherein X is N or —C(R⁴⁴)— where R⁴⁴ is R²² or R⁴⁴ and R²¹ are linked to form the bivalent radical —(CH₂)ₖ—, said heterocyclic is linked in the (i), (ii) or (iii) position to the U¹ or U² group as above defined. In one embodiment, the heterocyclic is linked in the (i) position. In another embodiment, the heterocyclic is linked in the (ii) or (iii) position. When present, the R²² group or groups may be attached at any otherwise vacant or unoccupied position on the ring, for example an R²² group may be attached at the (ii) position.

When R¹³ is a heterocyclic group having the following structure:

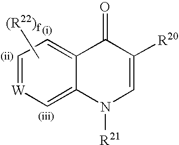

wherein W is N or —C(R⁴)— where R⁴⁴ is R²², said heterocyclic is linked in the (i), (ii) or (iii) position to the U¹ or U² group as above defined. In one embodiment, the heterocyclic is linked in the (i) or (iii) position. In another embodiment, the heterocyclic is linked in the (ii) position. When present, the R²² group or groups may be attached at any otherwise vacant or unoccupied position on the ring.

When R¹³ is a heterocyclic group having the following structure:

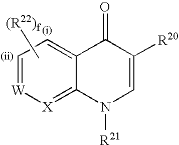

wherein W is N or —C(R⁴⁴)— where R⁴⁴ is R²² and X is —C(R⁴⁴>where R⁴⁴ is R²² or R⁴⁴ and R²¹ are linked to form the bivalent radical —(CH₂)ₖ—, or W is —C(R⁴⁴)— where R⁴⁴ is R²² and X is N, said heterocyclic is linked in the (i) or (ii) position, for example the (ii) position, to the U¹ or U² group as above defined. When present, the R²² group or groups may be attached at any otherwise vacant or unoccupied position on the ring.

When R¹³ is a heterocyclic group having the following structure:

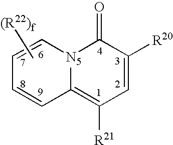

said heterocyclic is linked in the 6, 7, 8 or 9 position to the U¹ or U² group as above defined. In one embodiment, the heterocyclic is linked in the 7 or 8 position. In another embodiment, the heterocyclic is linked in the 6 or 9 position. When present, the R²² group or groups may be attached at any otherwise vacant or unoccupied position on the ring.

When R¹³ is a heterocyclic group having the following structure:

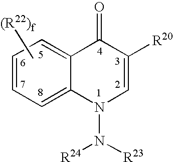

said heterocyclic is linked in the 5, 6, 7 or 8 position to the U¹ or U² group as above defined. In one embodiment, the heterocyclic is linked in the 6 or 7 position. When present, the R²² group or groups may be attached at any otherwise vacant or unoccupied position on the ring.

When R¹³ is a heterocyclic group having the following structure:

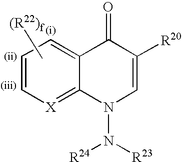

wherein X is —C(R⁴⁴)— where R⁴⁴ is R²², or R⁴⁴ and R²⁴ are linked to form a cyclic group of formula (IH), said heterocyclic is linked in the (i), (ii) or (iii) position to the U¹ or U² group as above defined. In one embodiment, the heterocyclic is linked in the (ii) or (iii) position.

When present, the R²² group or groups may be attached at any otherwise vacant or unoccupied position on the ring.

When R¹³ is a heterocyclic group having the following structure:

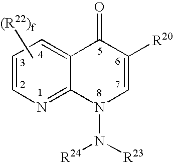

said heterocyclic is linked in the 2, 3 or 4 position to the U¹ or U² group as above defined.

In one embodiment, the heterocyclic is linked in the 2 or 3 position. When present, the R²² group or groups may be attached at any otherwise vacant or unoccupied position on the ring.

When R¹³ is a heterocyclic group having the following structure:

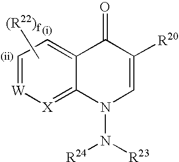

wherein W is N and X is —C(R⁴⁴)— where R⁴⁴ is R²² or R⁴⁴ and R²⁴ are linked to form a cyclic group of formula (IH), or W is —C(R²²)— and X is N or —C(R⁴⁴)— where R⁴⁴ is R²² or R⁴⁴ and R²⁴ are linked to form a cyclic group of formula (IH), said heterocyclic is linked in the (i) or (ii) position to the U¹ or U² group as above defined. In one embodiment, the heterocyclic is linked in the (ii) position. When present, the R²² group or groups may be attached at any otherwise vacant or unoccupied position on the ring.

When R¹³ is a heterocyclic group having the following structure:

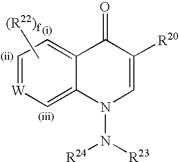

wherein W is N or —C(R²²)—, said heterocyclic is linked in the (i), (ii) or (iii) position to the U¹ or U² group as above defined. In one embodiment, the heterocyclic is linked in the (ii) position. When present, the R²² group or groups may be attached at any otherwise vacant or unoccupied position on the ring.

The term “alkyl” as used herein as a group or a part of a group refers to a straight or branched hydrocarbon chain containing the specified number of carbon atoms. For example, C₁₋₁₀alkyl means a straight or branched alkyl containing at least 1, and at most 10, carbon atoms. Examples of “alkyl” as used herein include, but are not limited to, methyl, ethyl, n-propyl, n-butyl, n-pentyl, isobutyl, isopropyl, t-butyl, hexyl, heptyl, octyl, nonyl and decyl. A C₁₋₄alkyl group is preferred, for example methyl, ethyl, n-propyl, isopropyl, n-butyl, isobutyl or t-butyl.

The term “C₃₋₇cycloalkyl” group as used herein refers to a non-aromatic monocyclic hydrocarbon ring of 3 to 7 carbon atoms such as, for example, cyclopropyl, cyclobutyl, cyclopentyl, cyclohexyl or cycloheptyl.

The term “alkoxy” as used herein refers to a straight or branched chain alkoxy group containing the specified number of carbon atoms. For example, C₁₋₆alkoxy means a straight or branched alkoxy containing at least 1, and at most 6, carbon atoms. Examples of “alkoxy” as used herein include, but are not limited to, methoxy, ethoxy, propoxy, prop-2-oxy, butoxy, but-2-oxy, 2-methylprop-1-oxy, 2-methylprop-2-oxy, pentoxy and hexyloxy. A C₁₋₄alkoxy group is preferred, for example methoxy, ethoxy, propoxy, prop-2-oxy, butoxy, but-2-oxy or 2-methylprop-2-oxy.

The term “alkenyl” as used herein as a group or a part of a group refers to a straight or branched hydrocarbon chain containing the specified number of carbon atoms and containing at least one double bond. For example, the term “C₂₋₆alkenyl” means a straight or branched alkenyl containing at least 2, and at most 6, carbon atoms and containing at least one double bond. Similarly, the term “C₃₋₆alkenyl” means a straight or branched alkenyl containing at least 3, and at most 6, carbon atoms and containing at least one double bond. Examples of “alkenyl” as used herein include, but are not limited to, ethenyl, 2-propenyl, 3-butenyl, 2-butenyl, 2-pentenyl, 3-pentenyl, 3-methyl-2-butenyl, 3-methylbut-2-enyl, 3-hexenyl and 1,1-dimethylbut-2-enyl. It will be appreciated that in groups of the form —O—C₂₋₆alkenyl, the double bond is preferably not adjacent to the oxygen.

The term “alkynyl” as used herein as a group or a part of a group refers to a straight or branched hydrocarbon chain containing the specified number of carbon atoms and containing at least one triple bond. For example, the term “C₃₋₆alkynyl” means a straight or branched alkynyl containing at least 3, and at most 6, carbon atoms and containing at least one triple bond. Examples of “alkynyl” as used herein include, but are not limited to, propynyl, 1-butynyl, 2-butynyl, 1-pentynyl and 3-methyl-1-butynyl.

The term “aryl” as used herein refers to an aromatic carbocyclic moiety such as phenyl, biphenyl or naphthyl, for example phenyl.

The term “heteroaryl” as used herein, unless otherwise defined, refers to an aromatic heterocycle of 5 to 10 members, having at least one heteroatom selected from nitrogen, oxygen and sulfur, and containing at least 1 carbon atom, including both mono and bicyclic ring systems. Examples of heteroaryl rings include, but are not limited to, furanyl, thiophenyl, pyrrolyl, pyrazolyl, imidazolyl, oxazolyl, isoxazolyl, thiazolyl, isothiazolyl, triazolyl, oxadiazolyl, tetrazolyl, thiadiazolyl, pyridyl, pyridazinyl, pyrazinyl, pyrimidinyl, triazinyl, quinolinyl, isoquinolinyl, 1,2,3,4-tetrahydroisoquinolinyl, benzofuranyl, benzimidazolyl, benzothienyl, benzoxazolyl, 1,3-benzodioxazolyl, indolyl, benzothiazolyl, furylpyridine, oxazolopyridyl and benzothiophenyl.

The term “5- or 6-membered heteroaryl” as used herein as a group or a part of a group refers to a monocyclic 5- or 6-membered aromatic heterocycle containing at least one heteroatom independently selected from oxygen, nitrogen and sulfur. Examples include, but are not limited to, furanyl, thiophenyl, pyrrolyl, pyrazolyl, imidazolyl, oxazolyl, isoxazolyl, thiazolyl, isothiazolyl, triazolyl, oxadiazolyl, tetrazolyl, pyridyl, pyridazinyl, pyrazinyl, pyrimidinyl and triazinyl.

The term “9- or 10-membered fused bicyclic heteroaryl” as used herein as a group or a part of a group refers to a 9- or 10-membered fused bicyclic heteroaryl containing at least one heteroatom selected from oxygen, nitrogen and sulphur. Examples include, but are not limited to, quinolinyl, isoquinolinyl, 1,2,3,4-tetrahydroisoquinolinyl, benzofuranyl, benzimidazolyl, benzothienyl, benzoxazolyl, 1,3-benzodioxazolyl, indolyl, benzothiazolyl, furylpyridine, oxazolopyridyl and benzothiophenyl.

The term “heterocyclyl” as used herein, unless otherwise defined, refers to a monocyclic or bicyclic 3- to 10-membered saturated or non-aromatic, unsaturated hydrocarbon ring containing at least one heteroatom selected from oxygen, nitrogen and sulfur. Preferably, the heterocyclyl ring has five or six ring atoms. Examples of heterocyclyl groups include, but are not limited to, pyrrolidinyl, tetrahydrofuranyl, tetrahydrothiophenyl, imidazolidinyl, pyrazolidinyl, piperidyl, piperazinyl, morpholino, tetrahydropyranyl and thiomorpholino.

The term “5- or 6-membered heterocyclic group” as used herein as a group or part of a group refers to a monocyclic 5- or 6-membered saturated hydrocarbon ring containing at least one heteroatom independently selected from oxygen, nitrogen and sulfur. Examples of such heterocyclyl groups include, but are not limited to, pyrrolidinyl, tetrahydrofuranyl, tetrahydrothiophenyl, imidazolidinyl, pyrazolidinyl, piperidyl, piperazinyl, morpholino, tetrahydropyranyl and thiomorpholino.

The term “halogen” refers to a fluorine, chlorine, bromine or iodine atom, such as fluorine and chlorine.

The terms “optionally substituted phenyl”, “optionally substituted phenyl or benzyl”, “optionally substituted 5- or 6-membered heteroaryl”, “optionally substituted 9- or 10-membered fused bicyclic heteroaryl” or “optionally substituted 5- or 6-membered heterocyclic group” as used herein refer to a group which is substituted by 1 to 3 groups independently selected from halogen, C₁₋₄alkyl, C₁₋₄alkoxy, hydroxy, nitro, cyano, amino, C₁₋₄alkylamino or diC₁₋₄alkylamino, phenyl and 5- or 6-membered heteroaryl.

As the skilled person will appreciate, the compounds of formula (I) are derivatives of known 14- and 15-membered macrolides derived from erythromycin A that have antibacterial activity and contain a cladinose moiety with a hydroxy group at 4″ position. The 14- and 15-membered macrolides which may be derivatised according to the invention include, for example, the following:
azithromycin,
11-O-methyl-azithromycin,
azithromycin 11,12-carbonate,
6-O-methyl erythromycin A,
6-O-methyl erythromycin A 9-oxime,
6-O-methyl erythromycin A 9-O-alkyl-oximes,
erythromycin 9-oxime,
erythromycin 9-O-alkyl-oximes,
erythromcyin 11,12-carbonate,
erythromycin 9-oxime 11,12-carbonate,
6-O-methyl-11-deoxy-11-amino-erythromycin A 11,12-carbamate,
(9S)-9-dihydro-erythromycin, and
(9S)-9-dihydro-erythromycin 9,11-ethylidene acetal.

In the compounds of formula (I), the heterocyclic group R¹³ is attached to the 4″ position of the 14- or 15-membered macrolide via a linker chain. Linker chains suitable for use according to the present invention include, for example, the following:

### —OS(O)₂(CH₂)₂NH(CH₂)₃—;

### —OS(O)₂(CH₂)₂—O—(CH₂)₃—;

### —OS(O)₂(CH₂)₂NH(CH₂)₂NH—;

### —OS(O)₂(CH₂)₂NH(CH₂)₂S—;

### —OS(O)₂(CH₂)₂NH(CH₂)₂O—;

### —OS(O)₂(CH₂)₂NH(CH₂)₃O—;

### —OS(O)₂(CH₂)₂—O—(CH₂)₂—O—(CH₂)₂NH—;

### —OS(O)₂(CH₂)₂NH(CH₂)₂—O—(CH₂)₂NH—;

### —OS(O)₂(CH₂)₂NH(CH₂)₂—O—(CH₂)₂—O—(CH₂)₂—; and

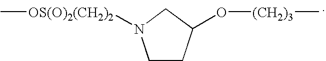

Further suitable linker chains include:

### —OS(O)₂(CH₂)₂NH(CH₃)(CH₂)₃—;

### —OS(O)₂(CH₂)₂—O—(CH₂)₂—O—(CH₂)₃—; and

### —OS(O)₂(CH₂)₂—O—(CH₂)₂—O—(CH₂)₂N(CH₃)—.

Representative examples of A include —C(O)—, —N(R⁷)—CH₂—, —C(═NR¹⁰)—, and A and R⁴ taken together with the intervening atoms forming a cyclic group having the following formula:

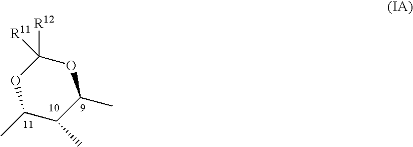

Representative examples of R¹ include:

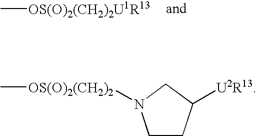

A representative example of R² is hydrogen.

Representative examples of R³ include hydrogen and C₁₋₄alkyl, such as hydrogen and methyl, for example hydrogen.

In one embodiment, R⁴ and R⁶ are hydroxy, R⁴ and A taken together with the intervening atoms form a cyclic group of formula (IA) and R⁵ is hydroxy, or R⁴ and R⁵ taken together with the intervening atoms form a cyclic group having the following structure:

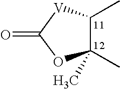

wherein V is a bivalent radical selected from —CH₂—, —CH(CN)—, —O—, —N(R¹⁶)— or —CH(SR¹⁵)—.

In a further embodiment, R⁴ and R⁵ are hydroxy.

A representative example of R³ is hydrogen.

A representative example of R⁷ is C₁₋₄alkyl, for example methyl.

In one embodiment, R¹¹ and R¹² are each independently hydrogen or C₁₋₆alkyl. In a further embodiment, one of R¹¹ and R¹² is hydrogen and the other is methyl.

Representative examples of R¹³ include heterocyclic groups having one of the following formulae:

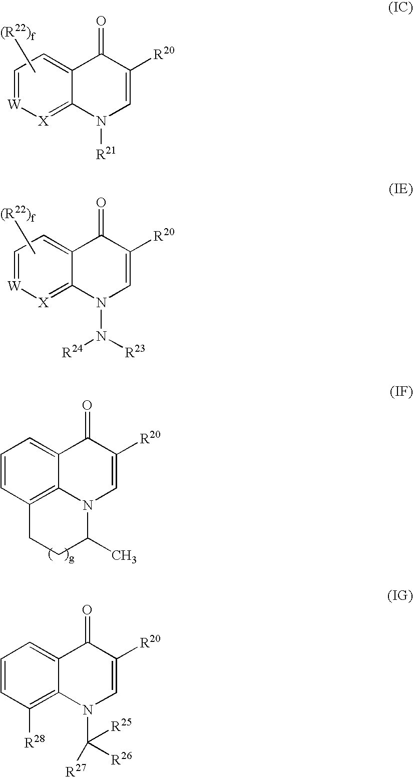

For example, R¹³ may be a heterocyclic group of formula (IC) or (IG).

In one embodiment, R¹⁶ is hydrogen.

Representative examples of R¹⁹ include hydrogen and C₁₋₄alkyl optionally substituted by —OR³⁵, for example hydrogen and methyl optionally substituted by —OR³⁵.

In one embodiment, R²⁰ is —C(O)OR³⁷, —C(O)NHR³⁷ or —C(O)CH₂NO₂. A representative example of R²⁰ is —C(O)OR³⁷.

Representative examples of R²¹ include C₁₋₄alkyl optionally substituted by up to three groups independently selected from hydroxyl, C₁₋₄alkoxy and halogen, for example methyl or ethyl optionally substituted by up to three fluorine atoms; C₁₋₄alkoxy, for example methoxy; C₂₋₆alkenyl, for example ethenyl; C₃₋₇cycloalkyl, for example cyclopropyl; and R²¹ and R⁴⁴ being linked to form the bivalent radical —(CH₂)ₖ—.

A representative example of R²² is halogen, for example fluorine or chlorine.

In one embodiment, R²³ and R²⁴ are each independently hydrogen or C₁₋₄alkyl, for example R²³ and R²⁴ are each hydrogen or R²³ and R²⁴ are each methyl. In another embodiment, R²³ and R²⁴, together with the nitrogen atom to which they are bound, form a five- to six-membered heterocyclic ring optionally containing one additional heteroatom selected from oxygen, sulfur and N—R⁴⁰, for example a six membered ring containing one additional heteroatom selected from oxygen, sulfur and N—R⁴⁰, such as morpholino.

In one embodiment, in the group (IG) one of R²⁵ and R²⁶ is hydrogen and the other is methyl.

In one embodiment, R²⁷ and R²⁸ are linked to form the bivalent radical —OCH₂— or —(CH₂)₂O—

A representative example of R³⁵ is C₁₋₄alkyl, for example methyl.

A representative example of R³⁷ is hydrogen.

A representative example of R⁴³ is hydrogen.

Representative examples of U¹ include —Y(CH₂)ₛB—, —Y(CH₂)ₛ—, —Y(CH₂)ₛB(CH₂)ₜD- and —Y(CH₂)ₛB(CH₂)ₜD(CH₂)ᵤ—, for example —Y(CH₂)ₛ—.

A representative example of U² is —Y(CH₂)ₛ—.

A representative example of V is —O—.

In one embodiment, W and X are each —C(R⁴⁴)—, W is —C(R⁴⁴)— and X is nitrogen or W is nitrogen and X is —C(R⁴⁴)—.

Representative examples of W include —CH—, —C(R²²)— and nitrogen.

Representative examples of X include —CH—, nitrogen and —C(R⁴⁴)— where R⁴⁴ and R²¹ are linked to form the bivalent radical —(CH₂)ₖ—.

Representative examples of Y include —N(R⁴³)— and —O—.

Representative examples of B include —N(R⁴³)—, —O— and —S—.

Representative examples of D include —N(R⁴³)— and —O—.

Representative examples of f include 0 and 1.

A representative example of g is 1.

A representative example of k is 3.

Representative examples of s include 2 and 3, for example 3.

A representative example of t is 2.

A representative example of u is 2.

A representative example of v is 0.

It is to be understood that the present invention covers all combinations of the embodiments and representative examples described hereinabove. It is also to be understood that the present invention encompasses compounds of formula (I) in which a particular group or parameter, for example R⁷, R¹⁶, R¹⁷, R¹⁸, R²², R²⁹, R³⁰, R³¹, R³², R³³, R³⁴, R³⁵, R³⁶, R³⁸, R³⁹, R⁴⁰, R⁴¹, R⁴², R⁴³, R⁴⁴, h, i, j, m, n, p, q and v may occur more than once. In such compounds it will be appreciated that each group or parameter is independently selected from the values listed.

Compounds of the invention include:
4″-O-{2-[3-(3-Carboxy-1,4-dihydro-1-ethyl-4-oxo-6-quinolinyl) propylamino]ethanesulfonyl}-6-O-methylerythromycin A,
4″-O-{2-[3-(3-Carboxy-1,4-dihydro-1-dimethylamino-4-oxo-6-quinolinyl)propylamino]ethanesulfonyl}-9-(S)-dihydroerythromycin A-9,11-ethylidene acetal,
4″-O-{2-[{2-(3-Carboxy-1,4-dihydro-1-ethyl-6-fluoro-4-oxo-[1,8]naphthyridin-7-ylamino)ethyl}amino]ethanesulfonyl}-6-O-methyl-erythromycin A formate,
4″-O-{2-[2-(3-Carboxy-1,4-dihydro-1-ethyl-4-oxo-[1,7]naphthyridin-6-ylsulfanyl)-ethylamino]ethanesulfonyl}-6-O-methyl-erythromycin A,
4″-O-{2-[3-(3-Carboxy-1,4-dihydro-1-dimethylamino-4-oxo-6-quinolinyl)propylamino]ethanesulfonyl}-6-O-methyl-erythromycin A,
4″-O-{2-[3-(3-Carboxy-1,4-dihydro-1-ethyl-4-oxo-[1,8]naphthyridin-6-yl)propylamino]ethanesulfonyl}-6-O-methylerythromycin A,
O-{2-[2-(3-Carboxy-1,4-dihydro-1-Dimethylamino-4-oxo-[1,7]naphthyridin-6-ylsulfanyl)-ethylamino]ethanesulfonyl}-6-O-methyl-erythromycin A formate,
4″-O-{2-[3-(6-Carboxy-7-oxo-2,3-dihydro-1H,7H-pyrido[3,2,1-ij]quinolin-9-yl)propylamino]ethanesulfonyl}-6-O-methylerythromycin A formate,
4″-O-{[(2-{[3-(6-Carboxy-3-methyl-7-oxo-1H,7H-[1,3]oxazino[5,4,3-ij]quinolin-9-yl)propyl]amino}ethyl)sulfonyl]-6-O-methylerythromycin A formate,
4″-O-{2-[3-(3-Carboxy-1,4-dihydro-1-(2,2,2-trifluoroethyl)-4-oxo-6-quinolinyl)propylamino]ethanesulfonyl}-6-O-methylerythromycin A,
4″-O-{2-[2-(3-Carboxy-1,4-dihydro-1-dimethylamino-4-oxo-6-quinolinyl)sulfanylethylamino]ethanesulfonyl}-6-O-methylerythromycin A,
4″-O-[(2-{[3-(7-Carboxy-8-oxo-3,4-dihydro-2H,8H-[1,4]oxazepino[2,3,4-ij]quinolin-10-yl)propyl]amino}ethyl)sulfonyl]-6-O-methylerythromycin A,
4″-O-[(2-{[3-(6-Carboxy-3,3-dimethyl-7-oxo-1H,7H-[1,3]oxazino[5,4,3-g]quinolin-9-yl)propyl]amino}ethyl)sulfonyl]-6-O-methylerythromycin A,
4″-O-{[2-({2-[(2-Carboxy-5-methyl-1-oxo-6,7-dihydro-1H,5H-pyrido[3,2,1-ij]quinolin-9-yl)thio]ethyl}amino)ethyl]sulfonyl}-6-O-methylerythromycin A,
4″-O-(2-[(2-{[6-carboxy-8-dimethylamino-5-oxo-5,8-dihydro-[1,8]naphthyridin-3-yl]thio}ethyl)amino]ethyl}sulfonyl)-6-O-methyl-erythromycin A formate,
4″-O-(2-({3-[6-Carboxy-3,3-dimethyl-7-oxo-1H,7H-[1,3]oxazino[5,4,3-ij]quinolin-9-yl]propyl}amino)ethanesulfonyl}-erythromycin A-(9E)-oxime,
4″-O-{[2-({2-[(2-Carboxy-5-methyl-1-oxo-6,7-dihydro-1H,5H-pyrido[3,2,1-ij]quinolin-9-yl)thio]ethyl}amino)ethyl]sulfonyl}erythromycin A-(9E)-oxime,
4″-O-{2-[3-(3-Carboxy-1,4-dihydro-1-(2-fluoroethyl)-4-oxo-4-quinolinyl)propylamino]ethanesulfonyl}-6-O-methylerythromycin A,
4″-O-{2-({3-[3-Carboxy-1-ethyl-4-oxo-1,4-dihydro-6-quinolinyl]propyl}amino)ethanesulfonyl}-erythromycin A-(9E)-oxime,
4″-O-{2-[3-(3-Carboxy-1,4-dihydro-1-dimethylamino-4-oxo-6-quinolinyl)propylamino]ethanesulfonyl}-azithromycin-11,12-carbonate,
4′-O-{2-[3-(3-Carboxy-1,4-dihydro-1-ethyl-4-oxo-6-quinolinyl) propylamino]ethanesulfonyl}-azithromycin-11,12-carbonate,
4″-O-{2-[3-(3-Carboxy-1,4-dihydro-1-ethyl-4-oxo-6-quinolinyl) propyloxy]ethanesulfonyl}-6-O-methylerythromycin A,
4″-O-{2-({3-[3-Carboxy-1-ethyl-4-oxo-1,4-dihydro-6-quinolinyl]propyl}amino)ethanesulfonyl}-erythromycin A-(9E)-O-methoxymethyloxime,
4″-O-{2-[3-(3-Carboxy-1,4-dihydro-1-Dimethylamino-4-oxo-6-quinolinyl)propylamino]ethanesulfonyl}-erythromycin A-(9E)-oxime-11,12-carbonate,
4″-O-{2-[3-(3-Carboxy-1,4-dihydro-1-ethyl-4-oxo-6-quinolinyl) propylamino]ethanesulfonyl}-erythromycin A-(9E)-oxime-11,12-carbonate,
4″-O-{2-[2-(3-Carboxy-1,4-dihydro-1-ethyl-6-fluoro-4-oxo-[1,8]naphthyridin-7-ylamino)ethylamino]ethanesulfonyl}-erythromycin A-(9E)-oxime-11,12-carbonate,
4″-O-{2-[3-(3-Carboxy-1,4-dihydro-1-(morpholin-4-yl)-4-oxo-6-quinolinyl)propylamino]ethanesulfonyl}-6-O-methylerythromycin A,
4″-O-{2-[3-(3-Carboxy-1,4-dihydro-1-dimethylamino-4-oxo-7-quinolinyl)oxyethylamino]ethanesulfonyl}-6-O-methylerythromycin A,
4″-O-{2-[(3R)-3-({3-[3-carboxy-1-ethyl-4-oxo-1,4-dihydro-6quinolinyl]propyl)oxy)-1-pyrrolidinyl]ethanesulfonyl}-erythromycin A-(9E)-oxime,
4″-O-{2-[3-(3-Carboxy-1,4-dihydro-1-dimethylamino-4-oxo-6-quinolinyl)propylamino]ethanesulfonyl}-azithromycin diformate salt,
4″-O-{2-[3-(3-Carboxy-1,4-dihydro-1-dimethylamino-4-oxo-6-quinolinyl)propylamino]ethanesulfonyl}-erythromycin A-11,12-carbonate,
4″-O-{2-[3-(3-Carboxy-1,4-dihydro-1-dimethylamino-4-oxo-7-quinolinyl)oxypropylamino]ethanesulfonyl}-6-O-methylerythromycin A,
4″-O-{2-[3-(3-Carboxy-1,4-dihydro-1-dimethylamino-4-oxo-6-quinolinyl)propyloxy]ethanesulfonyl}-6-O-methylerythromycin A,
4″-O-{2-[3-(3-Carboxy-1,4-dihydro-1-amino-4-oxo-7-quinolinyl)propylamino]ethanesulfonyl}-6-O-methylerythromycin A,
4″-O-{[2-({2-[(2-{[2-Carboxy-5-methyl-1-oxo-6,7-dihydro-1H,5H-pyrido[3,2,1-ij]quinolin-9-yl]amino}ethyl)oxy]ethyl}oxy)ethyl]sulfonyl}-6-O-methylerythromycin A,
4″-O-{[2-({2-[(2-{[2-Carboxy-5-methyl-1-oxo-6,7-dihydro-1H,5H-pyrido[3,2,1-ij]quinolin-9-yl]amino}ethyl)oxy]ethyl}amino)ethyl]sulfonyl)-6-O-methylerythromycin A,
4″-O-[(2-{[3-(6-Carboxy-3,3-dimethyl-7-oxo-2,3-dihydro-7H-[1,4]oxazino[2,3,4-ij]quinolin-9-yl)propyl]amino}ethyl)sulfonyl]-6-O-methyl erythromycin A,
4″-O-{2-[3-(3-Carboxy-1,4-dihydro-1-ethyl-4-oxo-6-quinolinyl)propylamino]ethanesulfonyl}-erythromycin A-11,12-carbonate,
4″-O-{2-[2-(3-Carboxy-1,4-dihydro-1-ethyl-6-fluoro-4-oxo-[1,8]naphthyridin-7-ylamino)ethylamino]ethanesulfonyl}-erythromycin A-11,12-carbonate,
4″-O-{2-[3-(3-Carboxy-1,4-dihydro-1-ethyl-4-oxo-6-qinolinyl)propylamino]ethanesulfonyl}-9-(S)-dihydroerythromycin A-9,11-ethylidene acetal,
4″-O-{2-[3-(6-Carboxy-7-oxo-1H,7H-[1,3]oxazino[5,4,3-ij]quinolin-9-yl)propylamino]ethanesulfonyl}-erythromycin A-(9E)-O-methoxymethyloxime,
4″-O-{2-[3-(3-Carboxy-1,4-dihydro-1-methyl-4-oxo-6-quinolinyl) propylamino]ethanesulfonyl}-6-O-methylerythromycin A,
4″-O-{2-[3-(3-Carboxy-1,4-dihydro-1-methyl-4-oxo-6-quinolinyl) propylamino]ethanesulfonyl}-erythromycin A-(9E)-oxime-11,12-carbonate,
4″-{2-[3-(3-Carboxy-1,4-dihydro-1-ethenyl-4-oxo-6-quinolinyl) propylamino]ethanesulfonyl}-6-O-methylerythromycin A,
4″-O-{2-{[2-({2-[(3-Carboxy-7-chloro-1-cyclopropyl-4-oxo-1,4-dihydro-6-quinolinyl)amino]ethyl}oxy)ethyl]oxy}ethanesulfonyl}-6-O-methylerythromycin A,
4″-O-{2-{[2-({2-[(3-Carboxy-7-chloro-1-cyclopropyl-4-oxo-1,4-dihydro-6-quinolinyl)amino]ethyl}oxy)ethy]oxy}ethanesulfonyl}-azithromycin,
4″-{2-(2-[(2-{[2-(3-Carboxy-1-ethyl-4-oxo-1,4-dihydro-6-quinolinyl)ethyl]oxy}ethyl)oxy]ethylamino)ethanesulfonyl}-erythromycin A-(9E)-O-methoxymethyloxime,
4″-O-{2-(2-{[(2-(3-Carboxy-1-ethyl-4-oxo-1,4-dihydro-6-quinolinyl)ethyl]oxy}ethyl)oxy]ethylamino)ethanesulfonyl}-6-O-methylerythromycin A,
4″-O-{2-[3-(3-Carboxy-1,4-dihydro-1-methoxy-4-oxo-6-quinolinyl) propylamino]ethanesulfonyl}-6-O-methylerythromycin A,
4″-O-{2-({3-[3-Carboxy-1-methoxy-4-oxo-1,4-dihydro-6-quinolinyl]propyl}amino)ethanesulfonyl}-erythromycin A-(9E)-O-methoxymethyloxime,
4″-O-{2-[2-(3-Carboxy-1,4-dihydro-1-ethyl-4-oxo-7-quinolinyloxy)ethylamino]ethanesulfonyl}-6-O-methylerythromycin A formate,
4″-O-{2-[2-(3-Carboxy-1,4-dihydro-1-ethyl-4-oxo-7-quinolinyloxy)ethylamino]ethanesulfonyl}erythromycin A-(9E)-oxime formate,
4″-O-{2-[2-(3-Carboxy-1,4Wihydro-1-ethyl-4-oxo-7-quinolinyloxy)ethylamino]ethanesulfonyl}erythromycin A-11,12-carbonate formate,
4″-O-{[2-({2-[(2-Carboxy-5-methyl-1-oxo-6,7-dihydro-1H,5H-pyrido[3,2,1-ij]quinolin-9-yl)amino]ethyl}amino)ethyl]sulfonyl}-6-O-methylerythromycin A,
4″-O-{2-[{2-(3-Carboxy-1,4-dihydro-1-ethyl-6-fluoro-4-oxo-[1,8]naphthyridin-7-ylamino)ethyl}amino]ethanesulfonyl-erythromycin A-(9E)-oxime,
4″-O-{2-[({2-(3-Carboxy-1,4-dihydro-1-ethyl-4-oxo-[1,8]naphthyridin-7-ylamino)ethyl}amino]ethanesulfonyl}-6-O-methylerythromycin A,
4″-O-{2-[3-(3-Carboxy-1,4-dihydro-1-ethyl-4-oxo-6-quinolinyl) propylamino]ethanesulfonyl}-erythromycin A (9E)-O-methyloxime,
4″-O-{2-[2-(3-Carboxy-1-ethyl-6-fluoro-4-oxo-1,4-dihydro-[1,8]naphthyridin-7-ylamino)ethyl}amino]ethanesulfonyl}-erythromycin A (9E)-O-methyloxime,
4″-O-{2-{2-({3-(3-Carboxy-1-ethyl-4-oxo-1,4-dihydro-6-quinolinyl)]propyl}oxy)ethylamino}ethanesulfonyl}-azithromycin triformate,
4″-O-{2-{[2-({3-[3-Carboxy-1-cyclopropyl-4-oxo-1,4-dihydro-6-quinolinyl]propyl}oxy)ethyl]oxy}ethanesulfonyl}-azithromycin,
4″-O-{2-{[2-({3-[3-Carboxy-1-ethyl-4-oxo-1,4-dihydro-6-quinolinyl]propyl}oxy)ethyl]oxy}ethanesulfonyl}-azithromycin,
4″-O-{2-([2-({2-[(3-Carboxy-1-cyclopropyl-4-oxo-1,4-dihydro-6-quinolinyl)amino]ethyl}oxy)ethyl]oxy}ethanesulfonyl}-azithromycin,
4″-O-{2-{[2-({2-[(3-Carboxy-1-cyclopropyl-4-oxo-1,4-dihydro-6-quinolinyl)methylamino]ethyl}oxy)ethyl]oxy}ethanesulfonyl}-azithromycin,
4″-O-{2-[2-(3-Carboxy-1,4-dihydro-1-ethyl-6-fluoro-4-oxo-[1,8]naphthyridin-7-ylamino)ethylamino]ethanesulfonyl}-erythromycin A-(9E)-O-cyanomethyloxime,
4″-O-{2-[3-(3-Carboxy-1,4-dihydro-1-dimethylamino-4-oxo-6-quinolinyl)propylamino]ethanesulfonyl}-erythromycin A-(9E)-O-(5-methylisoxazol-3-yl)-methyloxime,
4″-O-{2-[3-(3-Carboxy-1,4-dihydro-1-ethyl-4-oxo-4-quinolinyl)propylamino]ethanesulfonyl}-erythromycin A-(9E)-O(N,N-dimethylacetamido)-oxime,
4″-O-{2-({3-[3-carboxy-1-ethyl-4-oxo-1,4-dihydro-6-quinolinyl]propyl}amino)ethanesulfonyl}-erythromycin A (9E)-O-2-(diethylamino)ethyloxime,
4″-O-{2-[{2-(3-Carboxy-1,4-dihydro-1-ethyl-6-fluoro-4-oxo-[1,8]naphthyridin-7-ylamino)ethyl}amino]ethanesulfonyl}-erythromycin A (9E)-O-2-(diethylamino)ethyloxime,
4″-O-{2-[3-(3-Carboxy-1,4-dihydro-1-dimethylamino-4-oxo-6-quinolinyl)propylamino]ethanesulfonyl}-erythromycin A (9E)-2-(diethylamino)ethyloxime,
4″-O-[(2-{[3-(6-Carboxy-7-oxo-1H,7H-[1,3]oxazino[5,4,3-ij]quinolin-9-yl)propyl]amino}ethanesulfonyl]-erythromycin A (9E)-oxime,
4″-O-[(2-{[3-(6-carboxy-7-oxo-1H,7H-[1,3]oxazino[5,4,3-ij]quinolin-9-yl)propyl]amino}ethanesulfonyl]-erythromycin A (9E)-O-cyanomethyloxime,
4″-O-{2-[3-(7-Carboxy-8-oxo-3,4-dihydro-1H,8H-[1,4]oxazepino[6,5,4-ij]quinoline-10-yl)propylamino]ethanesulfonyl}-6-O-methyl-erythromycin A,
4″-O-{2-({3-[3-Carboxy-1-ethyl-4-oxo-1,4-dihydro-6-quinolinyl]propyl}methylamino)ethanesulfonyl}-erythromycin A-(9E)-oxime formate salt,
   and pharmaceutically acceptable derivatives thereof.

Compounds of the invention also include:
4″-O-{2-[3-(3-carboxy-1,4-dihydro-1-ethyl-4-oxo-6-quinolinyl) propylamino]ethanesulfonyl}-6-O-methylerythromycin A;
4″-O-{[(2-[3-(6-carboxy-3-methyl-7-oxo-1H,7H-[1,3]oxazino[5,4,3-ij]quinolin-9-yl)propyl]amino}ethyl)sulfonyl]-6-O-methylerythromycin A;
4″-O-2-[3-(3-carboxy-1,4-dihydro-1-methyl-4-oxo-6-quinolinyl) propylamino]ethanesulfonyl)-6-O-methylerythromycin A;
4″-O-{2-[3-(3-carboxy-1,4-dihydro-1-ethenyl-4-oxo-6-quinolinyl) propylamino]ethanesulfonyl}-6-O-methylerythromycin A;
4″-O-{2-{[2-({2-[(3-carboxy-7-chloro-1 cyclopropyl-4-oxo-1,4-dihydro-6-quinolinyl)amino]ethyl}oxy)ethyl]oxy}ethanesulfonyl}-azithromycin;
   and pharmaceutically acceptable derivatives thereof.

One or more compounds according to the invention exhibit antimicrobial activity, in particular antibacterial activity, against a range of clinical pathogenic microorganisms. Using a standard microtiter broth serial dilution test, one or more compounds of the invention have been found to exhibit useful levels of activity against a range of pathogenic microorganisims. For example, the compounds of the invention may be active against strains of Staphylococcus aureus, Streptopococcus pneumoniae, Moraxella catarrhalis, Streptococcus pyogenes, Haemophilus influenzae, Enterococcus faecalis, Chlamydia pneumoniae, Mycoplasma pneumoniae and Legionella pneumophila. The compounds of the invention may also be active against resistant strains, for example erythromycin resistant strains. Thus, for example, the compounds of the invention may be active against erythromycin resistant strains of Streptococcus pneumoniae, Streptococcus pyogenes and Staphylococcus aureus.

The compounds of the invention may therefore be used for treating a variety of diseases caused by pathogenic microorganisms, in particular bacteria, in human beings and animals. It will be appreciated that reference to treatment includes acute treatment or prophylaxis as well as the alleviation of established symptoms.

Thus, according to another aspect of the present invention we provide a compound of formula (I) or a pharmaceutically acceptable derivative thereof for use in therapy.

According to a further aspect of the invention we provide a compound of formula (I) or a pharmaceutically acceptable derivative thereof for use in the treatment or prophylaxis of systemic or topical microbial infections in a human or animal body.

According to a further aspect of the invention we provide the use of a compound of formula (I) or a pharmaceutically acceptable derivative thereof in the manufacture of a medicament for use in the treatment or prophylaxis of systemic or topical microbial infections in a human or animal body.

According to a yet further aspect of the invention we provide a method of treatment of the human or non-human animal body to combat microbial infections comprising administration to a body in need of such treatment of an effective amount of a compound of formula (I) or a pharmaceutically acceptable derivative thereof.

Infections include, but are not limited to, infections of soft tissue such as skin, which may include infections associated with acne and/or impetigo.

While it is possible that, for use in therapy, a compound of the invention may be administered as the raw chemical it is preferable to present the active ingredient as a pharmaceutical formulation e.g. when the agent is in admixture with a suitable pharmaceutical excipient, diluent or carrier selected with regard to the intended route of administration and standard pharmaceutical practice.

Accordingly, in one aspect, the present invention provides a pharmaceutical composition or formulation comprising a compound of the invention or a pharmaceutically acceptable derivative thereof in association with a pharmaceutically acceptable excipient, diluent and/or carrier. The excipient, diluent and/or carrier must be “acceptable” in the sense of being compatible with the other ingredients of the formulation and not deleterious to the recipient thereof.

In another aspect, the invention provides a pharmaceutical composition comprising, as active ingredient, a compound of the invention or a pharmaceutically acceptable derivative thereof in association with a pharmaceutically acceptable excipient, diluent and/or carrier for use in therapy, and in particular, in the treatment of human or animal subjects suffering from a condition susceptible to amelioration by an antimicrobial compound.

In another aspect, the invention provides a pharmaceutical composition comprising a therapeutically effective amount of the compounds of the present invention and a pharmaceutically acceptable excipient, diluent and/or carrier (including combinations thereof).

There is further provided by the present invention a process of preparing a pharmaceutical composition, which process comprises mixing a compound of the invention or a pharmaceutically acceptable derivative thereof, together with a pharmaceutically acceptable excipient, diluent and/or carrier.

The compounds of the invention may be formulated for administration in any convenient way for use in human or veterinary medicine and the invention therefore includes within its scope pharmaceutical compositions comprising a compound of the invention adapted for use in human or veterinary medicine. Such compositions may be presented for use in a conventional manner with the aid of one or more suitable excipients, diluents and/or carriers. Acceptable excipients, diluents and carriers for therapetic use are well known in the pharmaceutical art, and are described, for example, in Remington's Pharmaceutical Sciences, Mack Publishing Co. (A. R. Gennaro edit. 1985). The choice of pharmaceutical excipient, diluent and/or carrier can be selected with regard to the intended route of administration and standard pharmaceutical practice. The pharmaceutical compositions may comprise as—or in addition to—the excipient, diluent and/or carrier any suitable binder(s), lubricant(s), suspending agent(s), coating agent(s), solubilising agent(s).

Preservatives, stabilisers, dyes and even flavouring agents may be provided in the pharmaceutical composition. Examples of preservatives include sodium benzoate, sorbic acid and esters of p-hydroxybenzoic acid. Antioxidants and suspending agents may be also used.

For some embodiments, the agents of the present invention may also be used in combination with a cyclodextrin. Cyclodextrins are known to form inclusion and non-inclusion complexes with drug molecules. Formation of a drug-cyclodextrin complex may modify the solubility, dissolution rate, bioavailability and/or stability property of a drug molecule. Drug-cyclodextrin complexes are generally useful for most dosage forms and administration routes. As an alternative to direct complexation with the drug the cyclodextrin may be used as an auxiliary additive, e.g. as a carrier, diluent or solubiliser. Alpha-, beta- and gamma-cyclodextrins are most commonly used and suitable examples are described in WO 91/11172, WO 94/02518 and WO 98/55148.

The compounds of the invention may be milled using known milling procedures such as wet milling to obtain a particle size appropriate for tablet formation and for other formulation types. Finely divided (nanoparticulate) preparations of the compounds of the invention may be prepared by processes known in the art, for example see international patent application No. WO 02/00196 (SmithKline Beecham).

The routes for administration (delivery) include, but are not limited to, one or more of: oral (e.g. as a tablet, capsule, or as an ingestable solution), topical, mucosal (e.g. as a nasal spray or aerosol for inhalation), nasal, parenteral (e.g. by an injectable form), gastrointestinal, intraspinal, intraperitoneal, intramuscular, intravenous, intrauterine, intraocular, intradermal, intracranial, intratracheal, intravaginal, intracerebroventricular, intracerebral, subcutaneous, ophthalmic (including intravitreal or intracameral), transdermal, rectal, buccal, epidural and sublingual.

There may be different composition/formulation requirements depending on the different delivery systems. By way of example, the pharmaceutical composition of the present invention may be formulated to be delivered using a mini-pump or by a mucosal route, for example, as a nasal spray or aerosol for inhalation or ingestable solution, or parenterally in which the composition is formulated by an injectable form, for delivery, by, for example, an intravenous, intramuscular or subcutaneous route. Alternatively, the formulation may be designed to be delivered by both routes.

Where the agent is to be delivered mucosally through the gastrointestinal mucosa, it should be able to remain stable during transit though the gastrointestinal tract; for example, it should be resistant to proteolytic degradation, stable at acid pH and resistant to the detergent effects of bile.

Where appropriate, the pharmaceutical compositions can be administered by: inhalation; in the form of a suppository or pessary; topically in the form of a lotion, solution, cream, ointment or dusting powder; transdermally, for example, by a skin patch; orally in the form of tablets containing excipients such as starch or lactose, as capsules or ovules either alone or in admixture with excipients, or in the form of elixirs, solutions or suspensions containing flavouring or colouring agents; or parenterally, for example intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

The compositions of the invention include those in a form especially formulated for parenteral, oral, buccal, rectal, topical, implant, ophthalmic, nasal or genito-urinary use. For some applications, the agents of the present invention are delivered systemically (such as orally, buccally, sublingually), more preferably orally. Hence, preferably the agent is in a form that is suitable for oral delivery.

If the compound of the present invention is administered parenterally, then examples of such administration include one or more of: intravenously, intraarterially, intraperitoneally, intrathecally, intraventricularly, intraurethrally, intrasternally, intracranially, intramuscularly or subcutaneously administering the agent, and/or by using infusion techniques.

For parenteral administration, the compound is best used in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The compounds according to the invention may be formulated for use in human or veterinary medicine by injection (e.g. by intravenous bolus injection or infusion or via intramuscular, subcutaneous or intrathecal routes) and may be presented in unit dose form, in ampoules, or other unit-dose containers, or in multi-dose containers, if necessary with an added preservative. The compositions for injection may be in the form of suspensions, solutions, or emulsions, in oily or aqueous vehicles, and may contain formulatory agents such as suspending, stabilising, solubilising and/or dispersing agents. Alternatively the active ingredient may be in sterile powder form for reconstitution with a suitable vehicle, e.g. sterile, pyrogen-free water, before use.

The compounds of the invention can be administered (e.g. orally or topically) in the form of tablets, capsules, ovules, elixirs, solutions or suspensions, which may contain flavouring or colouring agents, for immediate-, delayed-, modified-, sustained-, pulsed- or controlled-release applications.

The compounds of the invention may also be presented for human or veterinary use in a form suitable for oral or buccal administration, for example in the form of solutions, gels, syrups, mouth washes or suspensions, or a dry powder for constitution with water or other suitable vehicle before use, optionally with flavouring and colouring agents. Solid compositions such as tablets, capsules, lozenges, pastilles, pills, boluses, powder, pastes, granules, bullets or premix preparations may also be used. Solid and liquid compositions for oral use may be prepared according to methods well known in the art. Such compositions may also contain one or more pharmaceutically acceptable carriers and excipients which may be in solid or liquid form.

Some compounds of the invention may be more suitable for a particular type of formulation/administration route than others.

Tablets may contain excipients such as microcrystalline cellulose, lactose, sodium citrate, calcium carbonate, dibasic calcium phosphate and glycine, disintegrants such as starch (preferably corn, potato or tapioca starch), sodium starch glycollate, croscarmellose sodium and certain complex silicates, and granulation binders such as polyvinylpyrrolidone, hydroxypropylmethylcellulose (HPMC), hydroxypropylcellulose (HPC), sucrose, gelatin and acacia.

Additionally, lubricating agents such as magnesium stearate, stearic acid, glyceryl behenate and talc may be included.

Solid compositions of a similar type may also be employed as fillers in gelatin capsules. Suitable excipients in this regard include lactose, starch, a cellulose, milk sugar or high molecular weight polyethylene glycols. For aqueous suspensions and/or elixirs, the agent may be combined with various sweetening or flavouring agents, colouring matter or dyes, with emulsifying and/or suspending agents and with diluents such as water, ethanol, propylene glycol and glycerin, and combinations thereof.

The compounds of the invention may also be administered orally in veterinary medicine in the form of a liquid drench such as a solution, suspension or dispersion of the active ingredient together with a pharmaceutically acceptable carrier or excipient.

The compounds of the invention may also, for example, be formulated as suppositories e.g. containing conventional suppository bases for use in human or veterinary medicine or as pessaries e.g. containing conventional pessary bases.

The compounds according to the invention may be formulated for topical administration, for use in human and veterinary medicine, in the form of ointments, creams, gels, hydrogels, lotions, solutions, shampoos, powders (including spray or dusting powders), pessaries, tampons, sprays, dips, aerosols, drops (e.g. eye ear or nose drops) or pour-ons.

For application topically to the skin, the agent of the present invention can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water.

Alternatively, it can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyidodecanol, benzyl alcohol and water.

The compounds may also be dermally or transdermally administered, for example, by use of a skin patch.

For ophthalmic use, the compounds can be formulated as micronised suspensions in isotonic, pH adjusted, sterile saline, or, preferably, as solutions in isotonic, pH adjusted, sterile saline, optionally in combination with a preservative such as a benzylalkonium chloride. Alternatively, they may be formulated in an ointment such as petrolatum.

As indicated, the compound of the present invention can be administered intranasally or by inhalation and is conveniently delivered in the form of a dry powder inhaler or an aerosol spray presentation from a pressurised container, pump, spray or nebuliser with the use of a suitable propellant, e.g. dichlorodifluoromethane, trichlorofluoromethane, dichlorotetrafluoroethane, a hydrofluoroalkane such as 1,1,1,2-tetrafluoroethane (HFA 134AT“ ”) or 1,1,1,2,3,3,3-heptafluoropropane (HFA 227EA), carbon dioxide or other suitable gas. In the case of a pressurised aerosol, the dosage unit may be determined by providing a valve to deliver a metered amount. The pressurised container, pump, spray or nebuliser may contain a solution or suspension of the active compound, e.g. using a mixture of ethanol and the propellant as the solvent, which may additionally contain a lubricant, e.g. sorbitan trioleate.

Capsules and cartridges (made, for example, from gelatin) for use in an inhaler or insufflator may be formulated to contain a powder mix of the compound and a suitable powder base such as lactose or starch.

For topical administration by inhalation the compounds according to the invention may be delivered for use in human or veterinary medicine via a nebuliser.

The compounds of the invention may also be used in combination with other therapeutic agents. The invention thus provides, in a further aspect, a combination comprising a compound of the invention or a pharmaceutically acceptable derivative thereof together with a further therapeutic agent.

When a compound of the invention or a pharmaceutically acceptable derivative thereof is used in combination with a second therapeutic agent active against the same or different disease state the dose of each compound may differ from that when the compound is used alone. Appropriate doses will be readily appreciated by those skilled in the art. It will be appreciated that the amount of a compound of the invention required for use in treatment will vary with the nature of the condition being treated and the age and the condition of the patient and will be ultimately at the discretion of the attendant physician or veterinarian. The compounds of the present invention may for example be used for topical administration with other active ingredients such as corticosteroids or antifungals as appropriate.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations by any convenient route.

When administration is sequential, either the compound of the invention or the second therapeutic agent may be administered first. When administration is simultaneous, the combination may be administered either in the same or different pharmaceutical composition.

When combined in the same formulation it will be appreciated that the two compounds must be stable and compatible with each other and the other components of the formulation. When formulated separately they may be provided in any convenient formulation, conveniently in such manner as are known for such compounds in the art. The compositions may contain from 0.01-99% of the active material. For topical administration, for example, the composition will generally contain from 0.01-10%, more preferably 0.01-1% of the active material.

Typically, a physician will determine the actual dosage which will be most suitable for an individual subject. The specific dose level and frequency of dosage for any particular individual may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy.

For oral and parenteral administration to humans, the daily dosage level of the agent may be in single or divided doses.

For systemic administration the daily dose as employed for adult human treatment it will range from 2-100 mg/kg body weight, preferably 5-60 mg/kg body weight, which may be administered in 1 to 4 daily doses, for example, depending on the route of administration and the condition of the patient. When the composition comprises dosage units, each unit will preferably contain 200 mg to 1 g of active ingredient. The duration of treatment will be dictated by the rate of response rather than by arbitrary numbers of days.

Compounds of general formula (I) and pharmaceutically acceptable derivatives thereof may be prepared by the general methods outlined hereinafter, said methods constituting a further aspect of the invention. In the following description, the groups R¹ to R⁴⁴, U¹, U², V, W, X, Y, B, D, E, d, e, f, g, h, i, j, k, m, n, p, q, r, s, t, u and v have the meaning defined for the compounds of formula (I) unless otherwise stated.

The groups U^{1z}R^{13z}, U^{2z}R^{13z} and R^{13z} are U²R¹³, U²R¹³ and R¹³ as defined for formula (I) or groups convertible to U¹R¹³, U²R¹³ and R¹³. Conversion of a group U^{1z}R^{13z}, U^{2z}R¹³ or R^{13z} to a U¹R¹³, U²R¹³ or R¹³ group typically arises if a protecting group is needed during the reactions described below. A comprehensive discussion of the ways in which such groups may be protected and methods for cleaving the resulting protected derivatives is given by for example T. W. Greene and P. G. M Wuts in Protective Groups in Organic Synthesis 2^{nd} ed., John Wiley & Son, Inc 1991 and by P. J. Kocienski in Protecting Groups, Georg Thieme Verlag 1994 which are incorporated herein by reference. Examples of suitable amino protecting groups include acyl type protecting groups (e.g. formyl, trifluoroacetyl and acetyl), aromatic urethane type protecting groups (e.g. benzyloxycarbonyl (Cbz) and substituted Cbz, and 9-fluorenylmethoxycarbonyl (Fmoc)), aliphatic urethane protecting groups (e.g. t-butyloxycarbonyl (Boc), isopropyloxycarbonyl and cyclohexyloxycarbonyl) and alkyl type protecting groups (e.g. benzyl, trityl and chlorotrityl). Examples of suitable oxygen protecting groups may include for example alkyl silyl groups, such as trimethylsilyl or tert-butyldimethylsilyl; alkyl ethers such as tetrahydropyranyl or tert-butyl; or esters such as acetate. Hydroxy groups may be protected by reaction of for example acetic anhydride, benzoic anhydride or a trialkylsilyl chloride in an aprotic solvent. Examples of aprotic solvents are dichloromethane, N,N-dimethylformamide, dimethylsulfoxide, tetrahydrofuran and the like.

The compounds of general formula (I) and derivatives thereof may be purified by conventional methods known in the art. For example, the compounds may be purified by HPLC using an aqueous solution of an acid such as formic acid or trifluoroacetic acid with an organic co-solvent such as acetonitrile or methanol.

In one embodiment of the invention, compounds of formula (I) wherein R¹ is —OS(O)₂(CH₂)₂U¹R³ where U¹ is as above defined and Y is —N(R⁴³)— or —S—, or compounds of formula (I) wherein R¹ is:

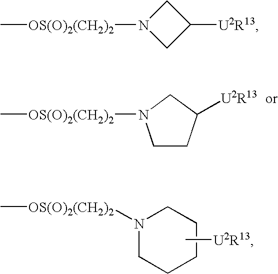

may be prepared by Michael reaction of a compound of formula (II), wherein R² is optionally a hydroxyl protecting group

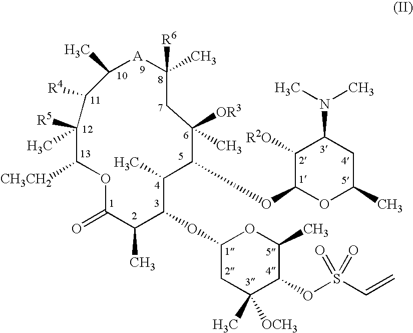

with a compound of formula HU^{1z}R^{13z} (III) or an amine (IV), (IVA) or (IVB)

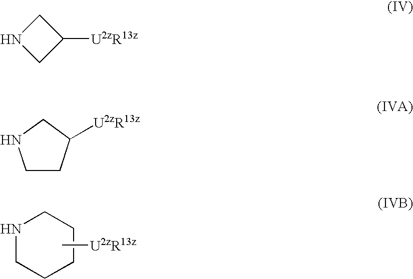

The reaction is suitably carried out in a solvent such as dimethylsulfoxide, N,N-dimethylformamide, 1-methyl-pyrrolidinone, a halohydrocarbon (e.g. dichloromethane), an ether (e.g. tetrahydrofuran or dimethoxyethane), acetonitrile or alcohol (e.g methanol or isopropanol) and the like, and in the presence of a base, followed, if desired, by removal of hydroxyl protecting group R² and conversion of the U^{1z}R^{13z} or U^{2z}R^{3z} group to U¹R¹³ or U²R¹³. Similarly, compounds of formula (I) wherein R¹ is —OS(O)₂(CH₂)₂U¹R¹³ where U¹ is as above defined and Y is —O— may also be prepared by Michael reaction in a solvent such as dimethylsulfoxide, N,N-dimethylformamide, 1-methyl-pyrrolidinone, a halohydrocarbon (e.g. dichloromethane), an ether (e.g. tetrahydrofuran or dimethoxyethane) or acetonitrile, and in the presence of a base.

Compounds of formula (I) may be converted into other compounds of formula (I). Thus compounds of formula (I) wherein Y is —S(O)ᵥ— and v is 1 or 2 may be prepared by oxidation of the corresponding compound of formula (I) wherein v is 0. The oxidation is preferably carried out using a peracid, e.g. peroxybenzoic acid, followed by treatment with a phosphine, such as triphenylphosphine. The reaction is suitably carried out in an organic solvent such as methylene chloride. Compounds of formula (I) wherein Y is —N(R⁴³)— and R⁴³ is C₁₋₄alkyl can be prepared from compounds wherein R⁴³ is hydrogen by reductive alkylation. Compounds of formula (I) wherein Y is —N(R⁴³)— and R⁴³ is acetyl or benzoyl can be prepared from compounds wherein R⁴³ is hydrogen by acylation.

Compounds of formula (II) wherein A is —C(O)—, —N(R⁷)—CH₂— or —CH(NR⁸R⁹)—, R⁴ or R⁵ are hydroxy or R⁴ and R⁵ taken together with the intervening atoms form a cyclic group having the following structure:

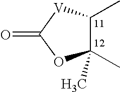

wherein V is a bivalent radical selected from —O— and —N(R¹⁵)—, and R³ is C₁₋₄alkyl, or C₃₋₆alkenyl optionally substituted by 9- or 10-membered fused bicyclic heteroaryl are known compounds or they may be prepared by analogous methods to those known in the art, such as the procedures described in EP 0 307 177, EP 0 248 279, WO 00/78773 and WO 97/42204.

Compounds of formula (II) wherein A is —N(CH₃)—CH₂—, R⁴ or R⁵ are hydroxy or R⁴ and R⁵ taken together with the intervening atoms form a cyclic group having the following structure:

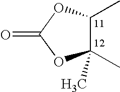

and R⁶ is hydrogen are known compounds or they may be prepared by analogous methods to those known in the art, such as the procedures described in EP 0 508 699, J. Chem. Res. Synop. (1988, pages 152-153) and U.S. Pat. No. 6,262,030.

Compounds of formula (II) wherein A is —C(═NR¹⁰)—, R⁴ or R⁵ are hydroxy or R⁴ and R⁵ taken together with the intervening atoms form a cyclic group having the following structure:

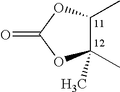

and R⁶ is hydrogen, are known compounds or they may be prepared by analogous methods to those known in the art. Thus they can be prepared according to the procedures described in EP 0 284 203.

Compounds of formula (II) wherein A is —C(O)—, R⁴ and R⁵ taken together with the intervening atoms form a cyclic group having the following structure:

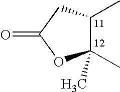

R⁶ is hydrogen and R³ is C₁₋₄alkyl may be prepared by decarboxylation of a compound of formula (V), wherein R⁴⁵ is a hydroxy protecting group followed, if required, by removal of the protecting group R² or R⁴⁵.

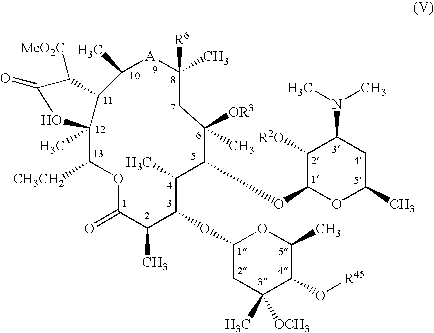

The decarboxylation may be carried out in the presence of a lithium salt such as lithium chloride, preferably in an organic solvent such as dimethylsulfoxide.

Compounds of formula (II) wherein A is —C(O)—, R⁴ and R⁵ taken together with the intervening atoms form a cyclic group having the following structure:

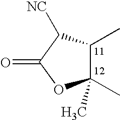

and R³ is C₁₋₄ alkyl may be prepared according to the procedures described in WO 02/50091 and WO 02/50092.

Compounds of formula (III) wherein U¹ is —Y(CH₂)₉B— in which B is —N(R⁴³)—, —O— or —S—, may be prepared by reaction of a compound of formula R^{13z}L (VI), wherein L is a suitable leaving group such as chlorine, fluorine or bromine, with a compound of formula —Y(CH₂)₅B— (VII) in which B is —N(R⁴³)—, —O— or —S—.

All publications, including but not limited to patents and patent applications, cited in this specification are herein incorporated by reference as if each individual publication were specifically and individually indicated to be incorporated by reference herein as though fully set forth.

In order that the invention may be more fully understood the following examples are given by way of illustration only.

The following abbreviations are used in the text: DCM for dichloromethane, DMF for N,N-dimethylformamide, DMSO for dimethyl sulfoxide, EtOAc for ethyl acetate, MeCN for acetonitrile, MeOH for methanol, Et₂O for diethyl ether, THF for tetrahydrofuran, TFA for trifluoroacetic acid, MgSO₄ for anhydrous magnesium sulphate, Na₂SO₄ for anhydrous sodium sulphate, SCX chromatography for strong cation exchange chromatography, and HPLC for high performance liquid chromatography.

### EXAMPLES

Erythromycin A (9E)-oxime may be prepared by the procedure described by R. R. Wilkening in EP 0 508 726 A1.

(9S)-9-O, 11-O-Ethylidene-9-dihydroerythromycin A may be prepared by the procedure described by E. Hunt et al. in J. Antibiotics, 1989, 42, 293-298.

2′-O-Acetyl-azithromycin-11,12-carbonate may be prepared by the procedure described by S. Djokic et al. in J. Chem. Research, Synopses, 1988, 5, 152-153.

Erythromycin A-(9E)-O-methoxymethyloxime may be prepared by the procedure described by Gasc, Jean Claude et al. in Journal of Antibiotics, 1991, 44(3), 313-30.

2′-O-Acetyl-erythromydin A-(9E)-O-acetyl-oxime may be prepared by the procedure described by J. Berge et. al. in WO 04/039822.

2′-O-Acetyl-azithromycin may be prepared by the procedure described by S. Djokic et al. in J. Chem. Research, Synopses, 1988, 5, 152-153.

2′-O-Acetyl-erythromycin A 11,12-carbonate may be prepared by the procedure described by L. Freidberg et al. in U.S. Pat. No. 4,686,207A.

Erythromycin A (9E)-O-methyloxime may be prepared by the procedure described by J. R. Everett et al in J. Chem. Soc. Perkin 2, 1989, 11, 1719-1728.

Erythromycin A-(9E)-O-(2-diethylaminoethyl)-oxime may be prepared by the procedure described by S Gouln d'Ambrieres et al. in U.S. Pat. No. 4,349,545.

Reverse phase HPLC refers to the use of a C18 column with a gradient of MeCN containing 0.1% TFA in water containing 0.1% TFA as eluent.

Mass directed automatic preparative HPLC refers to the use of a Waters Atlantis dC18 5 micron column with a gradient of MeCN containing 0.1% HCO₂H in H₂O containing 0.1% HCO₂H as eluent.

### Intermediate 1: 4″-O-Vinylsulfonyl-6-O-methyl-erythromycin A

To a stirred solution of 6-O-methyl-erythromycin A (8.94 g) in pyridine (180 mL) at 0° C. was added dropwise 2-chloro-1-ethanesulfonyl chloride (4.37 mL). After 25 min the mixture was allowed to warm to ca. 20° C. and after a further 60 min the mixture was poured into a 5% sodium bicarbonate solution. The mixture was extracted with DCM, and the organic extracts combined, dried (Na₂SO₄), filtered, and concentrated in vacuo to give a solid which was purified by flash chromatography (silica gel, 0-7% methanolic ammonia [2M] in DCM) to give the title compound as a white solid (2.57 g); ESMS m/z 838.5 [M+H]⁺.

### Intermediate 2: 4″-O-Vinylsulfonyl-(9S)-9-O, 11-O-ethylidene-9-dihydroerythromycin A

To a stirred solution of (9S)-9-O-11-C-ethylidene-9-dihydroerythromycin A (0.4 g) in pyridine (10 mL) at 0° C. was added dropwise 2-chloro-1-ethanesulfonyl chloride (0.06 mL). After 2 h the mixture was allowed to warm to ca. 20° C. and after a further 75 min a second aliquot of 2-chloro-1-ethanesulfonyl chloride (0.18 mL) was added. The mixture was stirred at ca. 20° C. for a further 2 h then concentrated in vacuo. The residue was taken up in DCM, washed with a 5% sodium bicarbonate solution, dried (Na₂SO₄), filtered, and concentrated in vacuo to give a solid which was purified by flash chromatography (silica gel, 0-5% methanolic ammonia [2M] in DCM) to give the title compound as a white solid (0.2 g); ESMS m/z 852.8 [M+H]⁺.

### Intermediate 3: 7-(2-Amino-ethylamino)-1-ethyl-6-fluoro-4-oxo-1,4-dihydro-[1,8]naphthyridine-3-carboxylic acid trifluoroacetate

### a) 7-(2-tert-Butoxycarbonylamino-ethylamino)-1-ethyl-6-fluoro-4-oxo-1,4-dihydro-[1,8]naphthyridine-3-carboxylic acid ethyl ester

7-Chloro-1-ethyl-6-fluoro-4-oxo-1,4-dihydro-[1,8]naphthyridine-3-carboxylic acid ethyl ester (2.2 g) in THF (20 mL) and MeCN (20 mL) was treated with triethylamine (3.07 mL), followed by (2-Amino-ethyl)-carbamic acid tert-butyl ester (1.41 g) and the mixture heated to 70° C. After 26 h additional (2-amino-ethyl)-carbamic acid tert-butyl ester (0.3 g) was added. After a further 15 h the heating was stopped and the solvent removed in vacuo. The residue was taken up in EtOAc, washed with water, dried (MgSO₄), filtered, and concentrated in vacuo to give a residue which was purified by flash chromatography (silica gel, 30-100% EtOAc in petroleum ether [b.p. 40-60° C.]) to give the title compound as a cream solid (2.89 g); ESMS m/z 423.0 [M+H]⁺.

### b) 7-(2-tert-Butoxycarbonylamino-ethylamino)-1-ethyl-6-fluoro-4-oxo-1,4-dihydro-[1,8]naphthyridine-3-carboxylic acid

To Intermediate 3a (2.89 g) in THF (30 mL) was added 2 N aqueous sodium hydroxide (3.4 mL), and the mixture stirred. After 24 h 2 N aqueous sodium hydroxide (0.6 mL) was added and stirring continued for a further 24 h. The solvent was then removed in vacuo, and the residue taken up in water (10 mL). Solid carbon dioxide was added, the resulting precipitate filtered off and dried in vacuo to give the title compound as a cream solid (2.65 g); ESMS m/z 395.1 [M+H]⁺.

### c) 7-(2-Amino-ethylamino)-1-ethyl-6-fluoro-4-oxo-1,4-dihydro-[1,8]naphthyridine-3-carboxylic acid trifluoroacetate

Intermediate 3b (2.65 g) was suspended in DCM (30 mL), TFA (15 mL) added, and the solution stirred for 35 min. The mixture was concentrated in vacuo, and again from toluene, and again from hexane to give the title compound as a tan powder (2.92 g); ESMS m/z 295.1 [M+H]⁺.

### Intermediate 4: 6-(3-Aminopropyl)-1-dimethylamino-4-oxo-1,4-dihydro-3-quinolinecarboxylic acid hydrochloride

### a) Ethyl 3-dimethylamino-2-(2-fluoro-5-iodobenzoyl)-2-propenoate

A stirred suspension of 2-fluoro-5-iodobenzoic acid (28.1 g) in DCM (300 mL) at 20° C. was treated with oxalyl chloride (13.9 mL) and DMF (5 drops). After 3 h the clear solution was evaporated and re-evaporated from toluene (2×). The acid chloride was re-dissolved in toluene (500 mL) and treated with triethylamine (22.5 mL) and ethyl 3-dimethylaminopropenoate (19.95 g); After stirring for 1.5 h at 90° C. the mixture was filtered and the solution purified by flash chromatography (silica gel, 40-70% EtOAc in petroleum ether [b.p. 40-60C]) to give the title compound as a yellow solid (30.8 g); APCI m/z 392.1 [M+H]⁺.

### b) Ethyl 3-(2,2-dimethylhydrazino)-2-(2-fluoro-5-iodobenzoyl)-2-propenoate

A stirred suspension of Intermediate 4a (28.2 g) in ethanol (300 mL) was treated with 1,1-dimethylhydrazine (4.76 mL). After stirring overnight the clear solution was evaporated to give the title compound as a yellow gum (29.6 g); APCI m/z 407.0 [M+H]⁺.

### c) Ethyl 1-dimethylamino-6-iodo-4-oxo-1,4-dihydro-3-quinolinecarboxylate

A mixture of Intermediate 4b (28.5 g) and potassium carbonate (14.5 g) in DMF (300 mL) was stirred at 100° C. for 1 h and then cooled. The mixture was poured into water, the solid filtered off then washed with water and dried to give the title compound as a white solid (22.8 g); APCI m/z 387.0 [M+H]⁺.

### d) 1-Dimethylamino-6-iodo-4-oxo-1,4-dihydro-3-quinolinecarboxylic acid

Intermediate 4c (12.8 g) was suspended in ethanol (130 mL) and treated with aqueous 1N sodium hydroxide (49.7 mL). The mixture was stirred overnight, diluted with 50% aqueous ethanol (200 mL) and heated at 50° C. for 4 h to complete the hydrolysis. The solution was evaporated to ca. 200 mL and then acidified. The solid was filtered off, washed with water and dried to give the title compound as a white solid (11 g); APCI m/z 359.0 [M+H]⁺.

### e) 6-(3-tert-Butoxycarbonylaminopropyn-1-yl)-1-dimethylamino-4-oxo-1,4-dihydro-3-quinolinecarboxylic acid

A stirred suspension of Intermediate 4d (20.46 g) and copper (I) iodide (1.08 g) in triethylamine (260 mL) and MeCN (380 mL) was degassed and covered with argon. After 15 min N-tert-butoxycarbonylpropargylamine (Casara, P. et. al. J. Chem. Soc. Perkin Trans. 1; 1985; 2201-2208) (10.6 g) and dichlorobis(triphenylphosphine)palladium (II) (1.26 g) were added. After 30 min the mixture was evaporated and redissolved in aqueous potassium carbonate (16 g in 300 mL). The mixture was washed with Et₂O (3×), filtered and acidified with citric acid. The solid was filtered off, washed with water and dried to give the title compound as a white solid (16.5 g); APCI m/z 386.0 [M+H]⁺.

### f) 6-(3-tert-Butoxycarbonylaminopropyl)-1-dimethylamino-4-oxo-1,4-dihydro-4-quinolinecarboxylic acid

A solution of Intermediate 4e (17.26 g) and sodium hydroxide (2.7 g) in MeOH (150 mL) and water (300 mL) was treated with 10% palladium on carbon (1 g) and hydrogenated at room temperature and atmospheric pressure overnight. The reaction mixture was filtered, acidified with citric acid, the solid filtered off, washed with water and dried to give the title compound as a white solid (16.2 g); APCI m/z 390.1 [M+H]⁺.

### g) 6-(3-Aminopropyl)-1-dimethylamino-4-oxo-1,4-dihydro-3-quinolinecarboxylic acid hydrochloride

A solution of Intermediate 4f (16.2 g) in DCM (500 mL) at ca. 20° C. was treated with 4M HCl in 1,4-dioxane (100 mL). After 1.5 h the solid was filtered off, washed with acetone and dried to yield the title compound as a white solid (13.5 g); APCI m/z 290.2 [M+H]⁺.

### Intermediate 5: 6-(3-Aminopropyl)-1-ethyl-4-oxo-1,4-dihydro-[1,8]-naphthyridine-3-carboxylic acid trifluoroacetate salt

### a) Ethyl 2-[(2-chloro-5-iodo-3-pyridinyl)carbonyl]-3-(ethylamino)-2-propenoate

5-Iodo-2-hydroxypyridine-3-carboxylic acid (T. R. Elworthy et al., J. Med. Chem., 40, 17, 1997, 2674-2687) (7.95 g) was suspended in thionyl chloride (40 mL). DMF (4 drops) was added and the mixture refluxed for 4 h. The resultant solution was evaporated to dryness. This acid chloride was then dissolved in 1,4-dioxane (40 mL) and added dropwise to a solution of ethyl 3-(ethylamino)-2-propenoate (5.15 g) and triethylamine (10.5 mL) in 1,4-dioxane at 0° C. After 1 h the cooling bath was removed, and the reaction stirred for 16 h. The mixture was then evaporated, saturated sodium hydrogen carbonate solution added, and extracted with EtOAc. The combined organic extracts were washed with brine, dried and concentrated in vacuo to give a dark oil which was purified by flash chromatography (silica gel, 33-5% Et₂O in petroleum ether [b.p. 40-60° C.]) to give the title compound as a pale brown solid (5.35 g); ESMS m/z 409.1 [M+H]⁺.

### b) Ethyl 1-ethyl-6-iodo-4-oxo-1,4-dihydro-[1,8]-naphthyridine-3-carboxylate

Intermediate 5a (4.92 g) was dissolved in DMF (50 mL), potassium carbonate (1.662 g) added, and the mixture heated at 50° C. for 16 h and 60° C. for 2 h. After evaporation the mixture was diluted with water and extracted with DCM. The combined organic extracts were dried and evaporated to give an oil. This was purified by flash chromatography (silica gel, 0-20% Et₂O in DCM) to give the title compound as a pale yellow solid (4.33 g); ESMS m/z 373.2 [M+H]⁺.

### c) Ethyl 6-[3-(tert-butoxycarbonylamino)-1-propyn-1-yl]-1-ethyl-4-oxo-1,4-dihydro-[1,8]-naphthyridine-3-carboxylate

A mixture of Intermediate 5b (2.176 g), copper (I) iodide (0.115 g) and triethylamine (27.9 mL) were suspended in dry MeCN (40 mL). The light green suspension was heated to 43° C. whilst argon was bubbled through. After 30 min, dichlorobis-(triphenylphosphine)palladium (II) (0.127 g) and N-tert-butoxycarbonylpropargylamine (1.542 g) were added and the mixture was heated at 43° C. for 25 min. The reaction mixture was cooled, filtered and concentrated in vacuo. The residue was taken up in DCM and washed with water. The organic phase was dried and concentrated in vacuo to provide a dark solid which was purified by flash chromatography (silica gel, 0-25% {MeOH: Et₂O [1:24]} in DCM) to give the title compound as a pale yellow solid (1.8 g); ESMS m/z 400.3 [M+H]⁺.

### d) Ethyl 6-[3-(tert-butoxycarbonylamino)-1-propyl]-1-ethyl-4-oxo-1,4-dihydro-[1,8]-naphthyridine-3-carboxylate

Intermediate 5c (0.91 g) in DCM (50 mL) was treated with 10% palladium on carbon (0.06 g) and hydrogenated at room temperature and atmospheric pressure for 75 min. The reaction mixture was filtered and concentrated, and the residue purified by flash chromatography (silica gel, 0-4% MeOH in DCM) to give the title compound as an off-white solid (0.83 g); ESMS m/z 404.3 [M+H]⁺.

### e) 6-[3-(tert-Butoxycarbonylamino)-1-propyl]-1-ethyl-oxo-1,4-dihydro-[1,8]-naphthyridine-3-carboxylic acid

Intermediate 5d (0.825 g) was dissolved in THF (15 mL), 0.2N sodium hydroxide (15 mL) added, and the reaction stirred for 16 h. The mixture was evaporated to a small volume then solid carbon dioxide added. The precipitate which formed was filtered off, washed well with water, and dried in vacuo over phosphorous pentoxide to give the title compound as an off-white powder (0.709 g); ESMS m/z 376.3 [M+H]⁺.

### f) 6-(3-Aminopropyl)-1-ethyl-4-oxo-1,4-dihydro-[1,8]-naphthyridine-3-carboxylic acid trifluoroacetate salt

Intermediate 5e (0.72 g) was dissolved in DCM (12 mL), TFA (4 mL) was added and the reaction stirred under argon for 0.75 h. The solution was evaporated to dryness, and the residue triturated with Et₂O to give, after drying, the title compound as an off-white powder (0.859 g); δ_{H} (400 MHz; DMSO-d6) 1.43 (3H, t), 1.95 (2H, m), 2.84 (2H, m), 2.92 (2H, t), 4.68 (2H, q), 7.78 (3H, br s), 8.63 (1H, d), 8.95 (1H, d), 9.24 (1H, s) and 14.8 (1H, br s).

### Intermediate 6: 6-(3-Aminopropyl)-1-ethyl-4-oxo-1,4-dihydro-quinoline-3-carboxylic acid trifluoroacetate salt

### a) 1-Ethyl-6-Iodo-4-oxo-1,4-dihydro-quinoline-3-carboxylic acid ethyl ester

A mixture of 1,4-dihydro-6-iodo-4-oxo-quinoline-3-carboxylic acid (J. Ellis et al., Aust J. Chem., 1973, 26, 907) (3.15 g), potassium carbonate (6.9 g) and iodoethane (15.6 g) in dry DMF was heated at 70° C. with vigorous stirring. After 16 h the mixture was cooled and diluted with EtOAc. The resultant mixture was washed with water and the organic phase separated, dried and evaporated to yield the title compound as pale yellow solid; δ_{H} (400 MHz; CDCl₃) 1.41 (3H, t, J=7.1 Hz), 1.54 (3H, t, J=7.3 Hz), 4.23 (2H, q, J=7.2 Hz), 4.40 (2H, q, J=7.1 Hz), 7.20 (1H, d, J=8.9 Hz), 7.95 (1H, dd, J=2.1 & 8.9 Hz), 8.48 (1H, s), 8.86 (1H, d, J=2.1 Hz).

### b) 6-(3-tert-Butoxycarbonylamino-prop-1-ynyl)-1-ethyl-4-oxo-1,4-dihydro-quinoline-3-carboxylic acid ethyl ester

Intermediate 6a (0.371 g), copper (I) iodide (0.026 g) and triethylamine (6.16 mL) were suspended in dry MeCN (22 mL). The light green suspension was heated to 50° C. whilst argon was bubbled through. After 20 min, dichlorobis(triphenylphosphine)palladium (II) (0.026 g) and N-tert-butoxycarbonylpropargylamine (0.264 g) were added and the brown suspension was heated under reflux. After 2 h the reaction mixture was cooled, filtered and concentrated. The residue was taken up in DCM and washed with water. The organic phase was dried and concentrated to provide a brown oil which was purified by flash chromatography (silica gel, 0-2.5% [9:1 MeOH:20M aqueous ammonia] in DCM) to give the title compound as a yellow solid; ESMS m/z 399.2 [M+H]⁺.

### c) 6-(3-tert-Butoxycarbonylaminopropyl)-1-ethyl-4-oxo-1,4-dihydro-quinoline-3-carboxylic acid ethyl ester

Intermediate 6b (0.366 g) in DCM (10 mL) was hydrogenated over 10% palladium on charcoal (0.05 g) for 16 h. The resultant mixture was filtered and the solvent evaporated to give the title compound as a yellow oil; ESMS m/z 403.2 [M+H]⁺.

### d) 6-(3-Aminopropyl)-1-ethyl-4-oxo-1,4-dihydro-quinoline-3-carboxylic acid ethyl ester

To Intermediate 6c (0.355 g) was added TFA (5 mL). After 1 h the solvent was evaporated to yield the title compound as a yellow oil; ESMS m/z 303.2 [M+H}]⁺.

### e) 6-(3-Aminopropyl)-1-ethyl-4-oxo-1,4-dihydro-quinoline-3-carboxylic acid sodium salt

To a solution of Intermediate 6d (0.25 g) in THF (6 mL) was added 2M sodium hydroxide (0.42 mL). After stirring for 16 h the mixture was saturated with carbon dioxide and the solvent evaporated. The residue was treated with MeOH (40 mL), filtered and the solvent evaporated to yield the title compound as a yellow solid; ESMS m/z 275.2 [M+H]⁺.

### f) 6-(3-Aminopropyl)-1-ethyl-4-oxo-1,4-dihydro-quinoline-3-carboxylic acid trifluoroacetate salt

Intermediate 6e (0.06 g) was subjected to reverse phase HPLC purification to give the title compound as white solid; δ_{H} (400 MHz; DMSO-d₆) 1.54 (3H, t, J=7.2 Hz), 2.0-2.1 (2H, m), 2.9-3.0 (4H, m), 4.58 (2H, q, J=7.2 Hz), 7.85 (1H, dd, J=2.2 & 8.8 Hz), 7.96 (1H, d, J=8.8 Hz), 8.36 (1H, d, J=1.8 Hz), 8.97 (1H, s).

### Intermediate 7: 9-(3-Amino-propyl)-1-oxo-6,7-dihydro-1H,5H-pyrido[3,2,1-ij]quinoline-2-carboxylic acid trifluoroacetate salt

### a) Diethyl 2-((3,4-dihydro-2H-quinolin-1-yl)methylene)malonate

A mixture of tetrahydroquinoline (13.32) and diethyl ethoxymethylenemalonate (21.62 g) was heated to 130° C. using a Dean-Stark apparatus. After 1 hour the reaction mixture was concentrated to give the title compound as a brown oil; ESMS m/z 304.3 [M+H]⁺.

### b) Ethyl 1-oxo-6,7-dihydro-1H,5H-pyrido[3,2,1-ij]quinoline-2-carboxylate

Intermediate 7a (2.5 g) was dissolved in polyphosphoric acid and the viscous mixture stirred for 4 hours at 110° C. The reaction mixture was cooled down before adding ice. The resulting precipitate was filtered off, washed with water then dried in a dessicator in the presence of phosphorous pentoxide to give the title compound as a beige solid; ESMS m/z 258.2 [M+H]⁺; δ_{H} (400 MHz; DMSO-d₆) 8.55 (s, 1H), 8.05 (dd, 1H), 7.54 (dd, 1H), 7.36 (dd, 1H), 4.27 (q, 2H), 4.22 (q, 2H), 3.00 (t, 2H), 2.10 (tt, 2H), 1.28 (t, 3H).

### c) Ethyl 9-bromo-1-oxo-6,7-dihydro-1H,5H-pyrido[3,2,1-ij]quinoline-2-carboxylate

Intermediate 7b (0.29 g) was dissolved in acetic acid (3 mL) and bromine (0.197 g) was added dropwise. The reaction was followed by LC/MS, additional bromine (2×0.197 g) was added. After 24 h water was added and the precipitate was filtered off, washed with Et₂O then dried in a dessicator in the presence of phosphorous pentoxide to provide an orange solid which was purified by flash chromatography (silica gel, 0-1.5% [9:1 MeOH:20M aqueous ammonia] in DCM) to give the title compound as a white solid; ESMS m/z 336.1/338.1 [M+H]⁺; δ_{H} (400 MHz; CDCl₃) δ 8.34 (d, 1H), 8.31 (s, 1H), 7.48 (d, 1H), 4.37 (q, 2H), 4.17 (t, 2H), 3.03 (t, 2H), 2.23 (ft, 2H), 1.40 (t, 3H).

### d) Ethyl 9-(3-tert-butoxycarbonylamino-prop-1-ynyl)-1-oxo-6,7-dihydro-1H,5H-pyrido[3,2,1-ij]quinoline-2-carboxylate

A yellow suspension of palladium acetate (0.073 g) and triphenylphosphine (0.191 g) in dry THF (6 mL) under argon was cooled to 0° C. A solution of n-butyllithium (2.5M in hexanes, 0.284 mL) was added dropwise and after 15 min the dark green suspension is warmed to ca. 20° C. for 15 mins. This suspension was then cannulated under argon into a white suspension of Intermediate 7c (0.337 g), copper (I) iodide (0.084 g) and N-tert-butoxycarbonylpropargylamine (0.198 g) in diethylamine (6 mL). The brown suspension was warmed to 45° C. for 2 h then filtered off, preabsorbed on silica gel and purified by flash chromatography (silica gel, 0-5% [9:1 MeOH:20M aqueous ammonia] in DCM) to give the title compound as a brown oil; ESMS m/z 411.3 [M+H]⁺; St (400 MHz; CDCl₃) 8.23 (s, 1H), 8.12 (d, 1H), 7.29 (d, 1H), 5.1 (m, 1H), 4.35 (q, 2H), 4.15 (m, 2×2H), 2.97 (t, 2H), 2.19 (ft, 2H), 1.49 (s, 9H), 1.38 (t, 3H).

### e) Ethyl 9-(3-tert-butoxycarbonylamino-propyl)-1-oxo-6,7-dihydro-1H,5H-pyrido[3,2,1-ij]quinoline-2-carboxylate

Intermediate 7d (0.318 g) was dissolved in DCM (50 mL), treated with 10% palladium on carbon (0.2 g) and hydrogenated at room temperature and atmospheric pressure overnight. The reaction mixture was filtered and concentrated to provide a brown oil which was purified by flash chromatography (silica gel, 0-1% [9:1 MeOH/20M aqueous ammonia] in DCM) to give the title compound as a brown oil; ESMS m/z 415.4 [M+H]⁺; δ_{H} (400 MHz; CDCl₃) 8.34 (s, 1H), 8.11 (bs, 1H), 7.25 (bs, 1H), 4.60 (m, 1H), 4.37 (q, 2H), 4.17 (t, 2H), 3.13 (q, 2H), 3.02 (t, 2H), 2.71 (t, 2H), 2.20 (ft, 2H), 1.85 (ft, 2H), 1.44 (s, 9H), 1.40 (t, 3H).

### f) 9-(3-tert-butoxycarbonylamino-propyl)-1-oxo-6,7-dihydro-1H,5H-pyrido[3,2,1-ij]quinoline-2-carboxylic acid sodium salt

Intermediate 7e (0.24 g) was dissolved in THF (3 mL) and treated with 2N aqueous sodium hydroxide (0.32 mL). The solution was heated to 50° C. overnight then treated with excess solid carbon dioxide. Evaporation of the solvent gave the title compound as a beige solid; ESMS m/z 387.2 [M+H]⁺; δ_{H} (400 MHz; DMSO-d₆) 8.83 (s, 1H), 8.11 (bs, 1H), 7.99 (s, 1H), 7.57 (s, 1H), 6.89 (bt, 1H), 4.41 (bt, 2H), 3.04 (t, 2H), 2.94 (q, 2H), 2.71 (t, 2H), 2.13 (m, 2H), 1.74 (m, 2H), 1.37 (s, 9H).

### g) 9-(3-Amino-propyl)-1-oxo-6,7-dihydro-1H,5H-pyrido[3,2,1-ij]quinoline-2-carboxylic acid trifluoroacetate salt

Intermediate 7f (0.224 g) was dissolved in TFA (3 mL). After 0.5 h the reaction mixture was concentrated to provide the title compound as a beige solid; ESMS m/z 287.2 [M+H]⁺; δ_{H} (400 MHz; CD₃OD) 8.83 (s, 1H), 8.15 (d, 1H), 7.62 (d, 1H), 4.43 (t, 2H), 3.14 (t, 2H), 2.98 (t, 2H), 2.89 (t, 2H), 2.66 (ft, 2H), 2.05 (ft, 2H).

### Intermediate 8: 6-(3-Aminopropyl)-1-(2,2,2-trifluoroethyl)-4-oxo-1,4-dihydro-quinoline-3-carboxylic acid trifluoroacetate salt

### a) 6-(3-tert-Butoxycarbonylamino-prop-1-ynyl)-1,4-dihydro-4-oxo-quinoline-3-carboxylic acid ethyl ester

A stirred mixture of 1,4-dihydro-6-iodo-4-oxo-quinoline-3-carboxylic acid ethyl ester (J. Tucker; V. Vaillancourt; J. Strohbach; K. Romines; M. Schnute; M. Cudahy; S. Thaisrivongs; and S. Turner, WO 99/32450) (1.97 g), copper (I) iodide (0.12 g), and triethylamine (25 mL) in dry DMF (50 mL) was heated to 50° C. whilst argon was bubbled through. After 10 min the mixture was cooled then treated with dichlorobis-(triphenylphosphine)palladium (II) (0.12 g) and N-tert-butoxycarbonylpropargylamine (1.34 g); The mixture was heated to 57° C. for 5.5 h, during which time further aliquots of N-tert-butoxycarbonylpropargylamine (0.11 g) and dichlorobis(triphenylphosphine)palladium (II) (0.02 g) were added. The mixture was then allowed to cool and the resulting precipitate filtered off, washed with diethyl ether, and dried in vacuo to yield the title compound as a cream solid (1.84 g); ESMS m/z 371.1 [M+H]⁺.

### b) 6-(3-tert-Butoxycarbonylaminopropyl)-1,4-dihydro-4-oxo-quinoline-3-carboxylic acid ethyl ester

A stirred solution of Intermediate 8a (1.0 g) in DCM:MeOH 3:1 (100 mL) was treated with 10% palladium on carbon (0.25 g) and hydrogenated at room temperature and atmospheric pressure for 2 h. The reaction mixture was filtered, and concentrated in vacuo to yield the title compound as a tan solid (1.01 g); ESMS m/z 375.1 [M+H]⁺.

### c) 6-(3-tert-Butoxycarbonylaminopropyl)-1-(2,2,2-trifluoroethyl)-4-oxo-1,4-dihydro-quinoline-3-carboxylic acid ethyl ester

To a stirred solution of Intermediate 8b (0.69 g) in DMF (7 mL) was added potassium carbonate (0.51 g), and 2,2,2-trifluoroethyl trifluoromethanesulfonate (0.47 g). After 6 h further aliquots of potassium carbonate (0.13 g), and 2,2,2-trifluoroethyl trifluoromethanesulfonate (0.13 g) were added. The mixture was stirred for a further 17 h then concentrated in vacuo to give a residue which was taken up in water and extracted with EtOAc. The organic extracts were combined, dried (MgSO₄), filtered, and concentrated in vacuo to give a residue which was purified by flash chromatography (silica gel, 0-4% methanolic ammonia [2M] in DCM) to give the title compound as a white solid (0.61 g); ESMS m/z 457.2 [M+H]⁺.

### d) 6-(3-tert-Butoxycarbonylaminopropyl)-1-(2,2,2-trifluoroethyl)-4-oxo-1,4-dihydro-quinoline-3-carboxylic acid

Intermediate 8c (0.61 g) was dissolved in THF (10 mL) and treated with 0.2N aqueous sodium hydroxide (8 mL). The solution was stirred for 18 h then concentrated in vacuo to give a residue which was taken up in water and treated with excess solid carbon dioxide. The resulting precipitate was removed by filtration and dried in vacuo to give the title compound as a white solid (0.53 g); ESMS m/z 429.2 [M+H]⁺.

### e) 6-(3-Aminopropyl)-1-(2,2,2-trifluoroethyl)-4-oxo-1,4-dihydro-quinoline-3-carboxylic acid trifluoroacetate salt

Intermediate 8d (0.53 g) was dissolved in DCM (9 mL) and treated with TFA (3 mL). After stirring for 35 min the mixture was concentrated in vacuo. Toluene and DCM were added to the residue and the mixture concentrated in vacuo to give the title compound as a white solid (0.612 g); ESMS m/z 329.3 [M+H]⁺.

### Intermediate 9: 6-(2-Aminoethylsulfanyl)-1-ethyl-4-oxo-1,4-dihydro-[1,7]-naphthyridine-3-carboxylic acid hydrochloride salt

### a) Ethyl 3-dimethylamino-2-(2,5-dichloroisonicotinoyl)-2-propenoate

A stirred suspension of 2,5-dichloroisonicotinic acid (1.49 g) in DCM (20 mL) at 20° C. was treated with oxalyl chloride (1 mL) and DMF (1 drop). After 1.5 h the clear solution was evaporated and re-evaporated from toluene (2×). The acid chloride was re-dissolved in toluene (50 mL) and treated with triethylamine (1.62 mL) and ethyl 3-dimethylaminopropenoate (1.44 g). After stirring for 1.5 h at 90° C. the mixture was filtered and the solution flash chromatographed (silica gel 50 to 70% EtOAc in petroleum ether [b.p. 40-60° C.]) to give the title compound as a yellow gum (2.3 g); APCI m/z 317.0, 319.0 [M+H]⁺.

### b) Ethyl 6-chloro-1-ethyl-4-oxo-1,4-dihydro-[1,7]-naphthyridine-3-carboxylate

A solution of Intermediate 9a (4.25 g) in ethanol (30 mL) at 20° C. was treated with 2M ethylamine in THF (8 mL) and stood overnight. The solution was evaporated and the residue redissolved in DMF (40 mL), treated with potassium carbonate (2.78 g) and stirred at 100° C. for 1 h. The mixture was cooled, filtered, evaporated to low volume and poured into water. The title compound was filtered off, washed with water and dried to give a yellow solid (2.12 g); APCI m/z 281.0, 283.0 [M+H]⁺.

### c) Ethyl 6-(2-tert-butoxycarbonylaminoethylsulfanyl)-1-ethyl-4-oxo-1,4-dihydro-[1,7]-naphthyridine-3-carboxylate

A stirred mixture of Intermediate 9b (1.5 g), tert-butyl 2-mercaptoethylcarbamate (1.1 mL) and potassium carbonate (1.1 g) In DMF (15 mL) was heated at 70° C. for 18 h. Further tert-butyl 2-mercaptoethylcarbamate (0.5 mL) was added and heating continued for 5 h. The mixture was cooled to 20° C., diluted with water and extracted with DCM (2×). The combined extracts were dried (Na₂SO₄) and evaporated. The residue was flash chromatographed (silica gel 30 to 50% EtOAc in DCM) to give the title compound as a yellow solid (1.33 g); APCI m/z 422.1 [M+H]⁺.

### d) 6-(2-tert-Butoxycarbonylaminoethylsulfanyl)-1-ethyl-4-oxo-1,4-dihydro-[1,7]-naphthyridine-3-carboxylic acid

Intermediate 9c (1.32 g) was suspended in MeOH (15 mL) and treated with 1N aqueous sodium hydroxide (4.7 mL) and water (10 mL). The mixture was stirred overnight. The solution was evaporated to low volume and then acidified with 5% citric acid. The solid was filtered off, washed with water and dried to give the title compound as a yellow solid (1.2 g); APCI m/z 394.1 [M+H]⁺.

### e) 6-(2-Aminoethylsulfanyl)-1-ethyl-4-oxo-1,4-dihydro-[1,7]-naphthyridine-3-carboxylic acid hydrochloride salt

A solution of Intermediate 9d (1.19 g) in DCM (20 mL) was treated with 4M HCl in 1,4-dioxan (10 mL). After 1.5 h the mixture was evaporated. The residue was triturated with acetone. The solid was filtered off, washed with acetone and dried to give the title compound as a yellow solid (1.02 g); APCI m/z 294.1 [M+H]⁺.

### Intermediate 10: 6-(2-Aminoethylsulfanyl)-1-dimethylamino-4-oxo-1,4-dihydro-[1,7]-naphthyridine-3-carboxylic acid hydrochloride salt

### a) Ethyl 6-chloro-1-dimethylamino-4-oxo-1,4-dihydro-[1,7]-naphthyridine-3-carboxylate

A solution of intermediate 9a (2.3 g) in ethanol (25 mL) was treated with 1,1-dimethylhydrazine (0.61 mL) and stood for 2 h. The solution was evaporated and the residue redissolved in DMF (20 mL), treated with potassium carbonate (1.09 g) and stirred at 100° C. for 1 h. The mixture was cooled, filtered, evaporated to low volume and poured into water. The title compound was filtered off, washed with water and dried to give a yellow solid (1.13 g); APCI m/z 296.0, 298.0 [M+H]⁺.

### b) Ethyl 6-(3-tert-butoxycarbonylaminoethylsulfanyl)-1-dimethylamino-4-oxo-1,4-dihydro-[1,7]-naphthyridine-3-carboxylate

A stirred mixture of Intermediate 10a (1.5 g), tert-butyl 2-mercaptoethylcarbamate (1.05 mL) and potassium carbonate (1.05 g) in DMF (15 mL) was heated at 70° C. for 18 h. Further tert-butyl 2-mercaptoethylcarbamate (0.5 mL) was added and heating continued for 5 h. The mixture was cooled to 20° C., diluted with water and extracted with DCM (2×). The combined extracts were dried (Na₂SO₄) and evaporated. The residue was flash chromatographed (silica gel 30-50% EtOAc in DCM) to give the title compound as a yellow solid (2.8 g); APCI m/z 437.1 [M+H]⁺.

### c) 6-(3-tert-Butoxycarbonylaminoethylsulfanyl)-1-dimethylamino-4-oxo-1,4-dihydro-[1,7]-naphthyridine-3-carboxylic acid

Intermediate 10b (2.8 g) was suspended in ethanol (20 mL) and treated with 1N aqueous sodium hydroxide (7.6 mL) and water (10 mL). The mixture was stirred overnight. The solution was evaporated to low volume and then acidified with 5% citric acid. The solid was filtered off, washed with water and dried to give the title compound as a yellow solid (1.2 g); APCI m/z 409.2 [M+H]⁺.

### d) 6-(2-Aminoethylsulfanyl)-1-dimethylamino-4-oxo-1,4-dihydro-[1,7]-naphthyridine-3-carboxylic acid hydrochloride salt

A solution of Intermediate 10c (1.19 g) in DCM (20 mL) at was treated with 4M HCl in 1,4-dioxan (5 mL). After 1.5 h the mixture was evaporated. The residue was triturated with acetone. The solid was filtered off, washed with acetone and dried to give the title compound as a yellow solid (0.95 g); APCI m/z 309.1 [M+H]⁺.

### Intermediate 11: 9-(3-Aminopropyl)-3-methyl-7-oxo-1H,7H-[1,3]oxazino[5,4,3-ij]quinoline-6-carboxylic acid trifloroacetate salt

### a) 2-[(2-Hydroxymethyl-4-iodo-phenylamino)-methylene]malonic acid diethyl ester

A mixture of (2-amino-5-iodo-phenyl)methanol (2.22 g) (C. Alabaster et al., J. Med. Chem., 1988, 31(10), 2048) and diethyl ethoxymethylenemalonate (1.93 g) was heated to 115° C. After 5 h the reaction mixture was concentrated and the residue was purified by chromatography on silica gel eluting with 0-5% [9:1 MeOH/20M aqueous ammonia] in DCM to give the title compound as a beige solid (3.43 g); ESMS m/z 420.0 [M+H]⁺.

### b) 2-[(2-Acetoxymethyl-4-iodo-phenylamino)-methylene]malonic acid diethyl ester

To a solution of Intermediate 11a (14.22 g) in triethylamine (15.5 mL) and DCM (150 mL) was added dropwise a solution of acetyl chloride (4.73 mL) in DCM (45 mL). After 1 h the solution was washed with water, the organic phase dried (MgSO₄), filtered, and concentrated in vacuo to give a residue which was purified by chromatography (silica gel, 0-6% Et₂O in DCM) to give the title compound as a pale yellow solid (14.58 g); ESMS m/z 462.1 [M+H]⁺.

### c) 8-Acetoxymethyl-6-iodo-4-oxo-1,4-dihydro-quinoline-3-carboxylic acid ethyl ester

A suspension of Intermediate 11b (14.57 g) and diphenyl ether (200 mL) was heated to 260° C. using a Dean-Stark apparatus. After the collection of ethanol was over the reaction mixture was allowed to cool down. The precipitate was filtered off, washed with diisopropyl ether then dried in a desiccator to give the title compound as a grey solid (9.8 g); ESMS m/z 416.0 [M+H]⁺.

### d) 8-Hydroxymethyl-6-iodo-4-oxo-1,4-dihydro-quinoline-3-carboxylic acid ethyl ester

A suspension of Intermediate 11c (1 g) and sodium ethoxide (0.165 g) in ethanol (50 mL) was heated at 80° C. for 2 h. The reaction mixture was allowed to cool down then the product was preabsorbed on silica gel and purified by chromatography eluting with 0 to 25% [9:1 MeOH/20M aqueous ammonia] in DCM to give the title compound as a beige solid (0.73 g); ESMS m/z 374.0 [M+H]⁺.

### e) 9-Iodo-3-methyl-7-oxo-1H,7H-[1,3]oxazino[5,4,3-ij]quinoline-6-carboxylic acid ethyl ester

A mixture of Intermediate 11d (0.50 g), 4-toluenesulfonic acid (0.211 g) and 1,1-diethoxyethane (1.4 mL) in N-methylpyrrolidinone (1 mL) was heated at 80° C. for 7 h. The reaction mixture was allowed to cool and diluted with chloroform (40 mL). The resultant solution was washed with 5% aqueous sodium hydrogen carbonate solution. The organic layer was separated, dried and evaporated to yield the title compound as a pale yellow solid (0.478 g); ESMS m/z 399.9 [M+H]⁺.

### f) 9-(3-tert-Butoxycarbonylamino-prop-1-ynyl)-3-methyl-7-oxo-1H,7H-[1,3]oxazino[5,4,3-ij]quinoline-6-carboxylic acid ethyl ester

A mixture of Intermediate 11e (0.478 g), copper (I) iodide (0.025 g) and triethylamine (5.8 mL) were suspended in dry MeCN (12 mL). The light green suspension was heated to 50° C. whilst argon was bubbled through. After 20 min, dichlorobis(triphenylphosphine) palladium (II) (0.025 g) and N-tert-butoxycarbonylpropargylamine (0.314 g) were added and the mixture was heated at 50° C. for 2 h. The reaction mixture was cooled, concentrated in vacuo and purified by chromatography (silica gel, 0 to 5% [9:1 MeOH/20 M aq. ammonia] in DCM to give the title compound as a beige solid (0.56 g); ESMS m/z 427.2 [M+H]⁺.

### g) 9-(3-tert-Butoxycarbonylamino-propyl)-3-methyl-7-oxo-1H,7H-[1,3]oxazino[5,4,3-ij]quinoline-6-carboxylic acid ethyl ester

A solution of Intermediate 11f (0.507 g) in DCM (30 mL) and was treated with 10% palladium on carbon (0.20 g) and hydrogenated at room temperature and atmospheric pressure for 17 h. The reaction mixture was filtered and concentrated to give the title compound as a yellow solid (0.53 g); ESMS m/z 431.2 [M+H]⁺.

### h) 9-(3-tert-Butoxycarbonylamino-propyl)-3-methyl-7-oxo-1H,7H-[1,3]oxazino[5,4,3-ij]quinoline-6-carboxylic acid sodium salt

A solution of Intermediate 11 g (0.51 g) in THF (10 mL) was treated with 2M sodium hydroxide (0.65 mL). After stirring at 50° C. for 17 h the mixture was treated with solid carbon dioxide and concentrated to give the title compound as a pale yellow solid (0.486 g); ESMS m/z 403.1 [M+H]⁺.

### l) 9-(3-Aminopropyl)-3-methyl-7-oxo-1H,7H-[1,3]oxazino[5,4,3-ij]quinoline-6-carboxylic acid trifloroacetate salt

A solution of Intermediate 11 h (0.486 g) in DCM (1 mL) was treated with trifluoroacetic acid (1 mL) and the reaction stirred at room temperature for 0.25 h. The solution was evaporated to dryness to give the title compound as a white solid (0.50 g); ESMS m/z 303.1 [M+H]⁺.

### Intermediate 12: 6-(3-Aminopropyl)-1-(morpholin-4-yl)-4-oxo-1,4-dihydro-3-quinolinecarboxylic acid hydrochloride

### a) Ethyl 3-dimethylamino-2-(2-fluoro-5-iodobenzoyl)-2-propenoate

A stirred suspension of 2-fluoro-5-iodobenzoic acid (28.1 g) In DCM (300 mL) at 20° C. was treated with oxalyl chloride (13.9 mL) and DMF (5 drops). After 3 h the clear solution was evaporated and re-evaporated from toluene (2×). The acid chloride was re-dissolved in toluene (500 mL) and treated with triethylamine (22.5 mL) and ethyl 3-dimethylaminopropenoate (19.95 g). After stirring for 1.5 h at 90° C. the mixture was filtered and the solution flash chromatographed on silica gel eluting with 40 to 70% EtOAc in petroleum ether [b.p. 40-60° C.] to give the title compound as a yellow solid (30.8 g); APCI m/z 392.1 [M+H]⁺.

### b) Ethyl 3-dimethylamino-2-{[5-(3-tert-butoxycarbonylamino)propyn-1-yl]-2-fluorobenzoyl}-2-propenoate

A stirred suspension of Intermediate 12a (2.64 g) and copper (I) iodide (0.129 g) in triethylamine (30 mL) and MeCN (60 mL) was degassed and covered with argon. After 15 min N-tert-butoxycarbonylpropargylamine (1.58 g) and dichlorobis(triphenylphosphine)-palladium (II) (0.15 g) were added. After 30 min the mixture was evaporated and redissolved in EtOAc. The mixture was washed with saturated sodium hydrogen carbonate solution, water (2×), dried (Na₂SO₄) and evaporated. The residue was flash chromatographed on silica gel eluting with 10 to 40% EtOAc in DCM to give the title compound as a yellow solid (2.97 g); APCI m/z 419.2 [M+H]⁺.

### c) Ethyl 2-(5-{3-[tert-butoxycarbonylamino]propyl}-2-fluorobenzoyl)-3-dimethylaminopropenoate

A solution of Intermediate 12b (3.97 g) in DCM (100 mL) was treated with 10% palladium on carbon (0.2 g). After 10 sec the solution was filtered and the catalyst replaced (0.3 g). The mixture was hydrogenated at room temperature and atmospheric pressure for 2 h, filtered and evaporated to give the title compound as a yellow solid (3.71 g);); APCI m/z 423.2 [M+H]⁺.

### d) Ethyl 6-(3-[tert-butoxycarbonylamino]propyl)-1-(morpholin-4-yl)-4-oxo-1,4-dihydro-3-quinolinecarboxylate

A mixture of Intermediate 12c (0.57 g), 4-aminomorpholine (0.152 g) and potassium carbonate (0.224 g) in DMF (10 mL) was stirred at 100° C. for 1 h and then cooled to 20° C. The mixture was diluted with EtOAc, washed with water (2×) and a solid crystallised out. The solid was filtered off, washed with EtOAc and dried to give the title compound as a white solid (0.445 g); APCI m/z 460.2 [M+H]⁺.

### e) 6-(3-tert-Butoxycarbonylaminopropyl)-1-(morpholin-4-yl)-4-oxo-1,4-dihydro-3-quinolinecarboxylic acid

Intermediate 12d (0.439 g) was suspended in THF (5 mL) and treated with 1N aqueous sodium hydroxide (1.43 mL) and water (5 mL). The mixture was stirred overnight. The solution was evaporated to ca. 5 mL and then acidified with citric acid. The solid was filtered off, washed with water and dried to give the title compound as a white solid (0.308 g); APCI m/z 432.0 [M+H]⁺.

### f) 6-(3-Aminopropyl)-1-(morpholin-4-yl)-4-oxo-1,4-dihydro-3-quinolinecarboxylic acid hydrochloride

A solution of Intermediate 12e (0.384 g) in DCM (5 mL) at ca. 20° C. was treated with 4M HCl in 1,4-dioxan (5 mL). After 1.5 h the mixture was evaporated. The residue was triturated with acetone. The solid was filtered off, washed with acetone and dried to yield the title compound as a white solid (0.325 g); APCI m/z 332.0 [M+H]⁺.

### Intermediate 13: 6-(2-Aminoethylsulfanyl)-1-dimethylamino-4-oxo-1,4-dihydro-quinoline-3-carboxylic acid trifluoroacetate salt

### a) Ethyl 2-[2,5-difluorobenzoyl]-3-dimethylamino-2-propenoate

A solution of 2,5-difluorobenzoyl chloride (5.26 g) in toluene (100 mL) was treated with ethyl-3-dimethylamino-2-propenoate (5.27 g), followed by triethylamine (5.9 mL). The mixture was stirred at 90° C. for 6.5 h then allowed to cool, and the precipitate removed by filtration. The filtrate was concentrated in vacuo to give a residue which was purified by flash chromatography (silica gel, 50-100% Et₂O in petroleum ether [b.p. 40-60° C.]) to give the title compound as a yellow oil (0.95 g); ESMS m/z 284.2 [M+H]⁺, 306.1 [M+Na]⁺.

### b) Ethyl 2-[2,5-difluorobenzoyl]-3-(2,2-dimethylhydrazino)-2-propenoate

A stirred solution of Intermediate 13a (0.93 g) in ethanol (10 mL) was treated with 1,1-dimethylhydrazine (0.27 mL). After 2 h a further aliquot of 1,1-dimethylhydrazine (0.05 mL) was added, and stirring continued for another 25 min. The mixture was concentrated in vacuo to give the title compound as a yellow oil (1.01 g); ESMS m/z 299.1 [M+H]⁺.

### c) Ethyl 1-dimethylamino-6-fluoro-4-oxo-1,4-dihydro-quinoline-3-carboxylate

A mixture of Intermediate 13b (0.98 g) and potassium carbonate (0.68 g) in DMF (10 mL) was stirred at 100° C. for 55 min and then cooled. The mixture was treated with water, the solid filtered off, washed with water then dried in vacuo to give the title compound as a pale yellow solid (0.63 g); ESMS m/z 279.2 [M+H]⁺.

### d) 6-(2-tert-Butoxycarbonylamino-ethylsulfanyl)-1-dimethylamino-4-oxo-1,4-dihydro-quinoline-3-carboxylic acid ethyl ester

A mixture of Intermediate 13c (1.0 g) and potassium carbonate (0.99 g) in DMSO (18 mL) was treated with tert-butyl N-(2-mercaptoethyl)-carbamate (1.3 mL) and heated to 70° C. After 21 h the mixture was allowed to cool then diluted with water and extracted with EtOAc. The organic extracts were combined, dried (MgSO₄), filtered, and concentrated in vacuo to give a residue which was purified by flash chromatography (silica gel, 20-100% EtOAc in petroleum ether [b.p. 40-60° C.]) to give the title compound as a white solid (1.08 g); ESMS m/z 436.2 [M+H]⁺.

### e) 6-(2-tert-Butoxycarbonylamino-ethylsulfanyl)-1-dimethylamino-4-oxo-1,4-dihydro-quinoline-3-carboxylic acid

Intermediate 13d (1.07 g) was dissolved in THF (16 mL) and treated with 0.2N aqueous sodium hydroxide (15 mL). The solution was stirred for 18.5 h then concentrated in vacuo to give a residue which was taken up in water and treated with excess solid carbon dioxide. The resulting precipitate was removed by filtration and dried in vacuo to give the title compound as a white solid (0.92 g); ESMS m/z 408.2 [M+H]⁺.

### f) 6-(2-Aminoethylsulfanyl)-1-dimethylamino-4-oxo-1,4-dihydro-quinoline-3-carboxylic acid trifluoroacetate salt

Intermediate 13e (0.9 g) was dissolved in DCM (20 mL) and treated with TFA (8 mL). After stirring for 30 min the mixture was concentrated in vacuo. Toluene and DCM were added to the residue and the mixture concentrated in vacuo. The resulting residue was triturated with Et₂O then dried in vacuo to give the title compound as a white solid (0.92 g); ESMS m/z 308.1 [M+H]⁺.

### Intermediate 14: 2-(3-Aminopropyl)-4-oxo-8,9-dihydro-4H,7H-10-oxa-6a-aza-cyclohepta[de]naphthalene-5-carboxylic trifluoroacetate salt

### a) 8-Fluoro-1-(3-hydroxypropyl)-6-iodo-4-oxo-1,4-dihydro-quinoline-3-carboxylic acid ethyl ester

A suspension of 8-fluoro-6-iodo-4-oxo-1,4-dihydro-quinoline-3-carboxylic acid ethyl ester (J. Tucker et al., WO 99/32450) (0.722 g), sodium iodide (0.3 g), sodium carbonate (0.636 g) and 3-bromopropanol (1.8 mL) in DMF (5 mL) was heated at 80° C. for 3 h. The reaction mixture was cooled, filtrated and concentrated in vacuo. The residue was preabsorbed on silica gel and purified by chromatography (silica gel, 0 to 1% [9:1 MeOH/20M aqueous ammonia] in DCM to give the title compound as a beige solid (0.62 g); ESMS m/z 420.1 [M+H]⁺.

### b) 2-Iodo-4-oxo-8,9-dihydro-4H,7H-10-oxa-6a-aza-cyclohepta[de]naphthalene-5-carboxylic acid ethyl ester

A solution of Intermediate 14a (0.575 g) and 1,8-diazabicyclo[5,4,0]undec-7-ene (0.205 mL) in DMF (14 mL) was heated at 100° C. for 17 h. The reaction mixture was concentrated in vacuo and the residue was purified by chromatography (silica gel, 0 to 5% [9:1 MeOH/20M aqueous ammonia] in DCM to give the title compound as a pale yellow solid (0.504 g); ESMS m/z 400.1 [M+H]⁺.

### c) 2-(3-tert-Butoxycarbonylamino-prop-1-ynyl)-4-oxo-8,9-dihydro-4H,7H-10-oxa-6a-aza-cyclohepta[de]naphthalene-5-carboxylic acid ethyl ester

A mixture of Intermediate 14b (0.493 g), copper (I) iodide (0.026 g) and triethylamine (6 mL) were suspended in dry MeCN (12 mL). The suspension was heated to 50° C. whilst argon was bubbled through. After 20 min, dichlorobis(triphenylphosphine)palladium (II) (0.026 g) and N-tert-butoxycarbonylpropargylamine (0.326 g) were added and the mixture was heated at 50° C. for 1.5 h. The reaction mixture was cooled, concentrated in vacuo and purified by chromatography (silica gel, 0 to 5% [9:1 MeOH/20M aqueous ammonia] in DCM to give the title compound as a beige solid (0.7 g); ESMS m/z 427.3 [M+H]⁺.

### d) 2-(3-tert-Butoxycarbonylamino-propyl)-4-oxo-8,9-dihydro-4H,7H-10-oxa-6a-aza-cyclohepta[de]naphthalene-5-carboxylic acid ethyl ester

A solution of Intermediate 14c (0.7 g) in DCM (150 mL) was treated with 10% palladium on carbon (0.3 g) and hydrogenated at ca. 20° C. and atmospheric pressure for 17 h. The reaction mixture was filtered, concentrated and purified by chromatography (silica gel, 0 to 5% [9:1 MeOH/20M aqueous ammonia] in DCM) to give the title compound as a pale yellow solid (0.5 g); ESMS m/z 431.4 [M+H]⁺.

### e) 2-(3-tert-Butoxycarbonylamino-propyl)-4-oxo-4,9-dihydro-4H,7H-10-oxa-6a-aza-cyclohepta[de]naphthalene-5-carboxylic acid sodium salt

A solution of Intermediate 14d (0.5 g) in THF (10 mL) and 1,4dioxan (12 mL) was treated with 0.2 N sodium hydroxide (0.961 mL). The reaction was stirred for 17 h at 50° C. then at 60° C. for 7 h. The reaction was cooled, solid carbon dioxide was added and the reaction mixture was concentrated to give the title compound as a pale yellow solid (0.66 g); ESMS m/z 403.3 [M+H]⁺.

### f) 2-(3-Amino-propyl)-4-oxo-8,9-dihydro-4H,7H-10-oxa-6a-aza-cyclohepta[de]naphthalene-5-carboxylic acid trifluoroacetate salt

A solution of Intermediate 14e (0.49 g) in DCM (10 mL) was treated with TFA (3 mL) and the reaction stirred for 0.33 h. The solution was evaporated to give the title compound as a beige solid (0.5 g); ESMS m/z 303.3 [M+H]⁺.

### Intermediate 15: 9-(3-Aminopropyl)-3,3-dimethyl-7-oxo-1H,7H-[1,3]oxazino[5,4,3-ij]quinoline-6-carboxylic acid trifluoroacetate salt

### a) 9-Iodo-3,3-dimethyl-7-oxo-1H,7H-[1,3]oxazino[5,4,3-ij]quinoline-6 carboxylic acid ethyl ester

A suspension of Intermediate 1d (3.45 g), p-toluene sulfonic acid (1.45 g) and dimethoxypropane (11.3 mL) in N-methylpyrrolidinone (8 mL) was heated at 80° C. for 3 h. The reaction mixture was concentrated and the residue partitioned between water and DCM. The organic extracts were combined, dried (MgSO₄), filtered, and concentrated in vacuo to give the title compound as an orange solid as a mixture with its methyl ester (3.18 g); ESMS m/z 414.0 and 400.0 [M+H]⁺.

### b) 9-(3-tert-Butoxycarbonylamino-prop-1-ynyl)-3,3-dimethyl-7-oxo-1H,7H-[1,3]oxazino[5,4,3-ij]quinoline-6-carboxylic acid ethyl ester

A mixture of Intermediate 15a (3.1 g), copper (I) iodide (0.166 g) and triethylamine (38 mL) were suspended in dry MeCN (75 mL). The suspension was heated to 50° C. whilst argon was bubbled through. After 20 min, dichlorobis(triphenylphosphine)palladium (II) (0.166 g) and N-tert-butoxycarbonylpropargylamine (2 g) were added and the mixture was heated at 50° C. for 1.5 h. The reaction mixture was cooled, concentrated in vacuo and purified by chromatography (silica gel, 0 to 5% [9:1 MeOH/20 M aq. ammonia] in DCM) to give the title compound as a beige solid as a mixture with its methyl ester (3 g); ESMS m/z 441.3 and 427.3 [M+H]⁺.

### c) 9-(3-tert-Butoxycarbonylaminopropyl)-3,3-dimethyl-7-oxo-1H,7H-[1,3]oxazino[5,4,3-ij]quinoline-6-carboxylic acid ethyl ester

A solution of Intermediate 15b (3 g) in DCM (100 mL) and MeOH (100 mL) was treated with 10% palladium on carbon (1 g) and hydrogenated at room temperature and atmospheric pressure for 65 h. The reaction mixture was filtered and concentrated to give the title compound as a beige solid as a mixture with its methyl ester (1.345 g); ESMS m/z 445.4 and 431.3 [M+H]⁺.

### d) 9-(3-tert-Butoxycarbonylaminopropyl)-3,3-dimethyl-7-oxo-1H,7H-[1,3]oxazino[5,4,3-ij]quinoline-6-carboxylic acid sodium salt

A solution of Intermediate 15c (1.345 g) in THF (30 mL) was treated with 0.2N sodium hydroxide (1.7 mL). The reaction was stirred for 17 h at 50° C. then solid carbon dioxide was added and the reaction mixture was concentrated to give the title compound as a white solid (1.4 g); ESMS m/z 417.3 [M+H]⁺.

### e) 9-(3-Aminopropyl)-3,3-dimethyl-7-oxo-1H,7H-[1,3]oxazino[5,4,3-ij]quinoline-6-carboxylic acid trifluoroacetate salt

A solution of Intermediate 15d (1.4 g) in DCM (50 mL) was treated with TFA (15 mL) and the reaction stirred at ca. 20° C. for 0.33 h. The solution was evaporated to dryness then purified by reverse phase HPLC to give the title compound as a beige solid (1.13 g); ESMS m/z 317.3 [M+H]⁺.

### Intermediate 16: 9-(2-Aminoethylsulfanyl)-5-methyl-1-oxo-6,7-dihydro-1H,5H-pyrido[3,2,1-ij]quinoline-2-carboxylic acid trifluoroacetate salt

### a) 9-Fluoro-5-methyl-1-oxo-6,7-dihydro-1H,5H-pyrido[3,2,1-ij]quinoline-2-carboxylic acid ethyl ester

A suspension of flumequine (1.045 g), ethyl iodide (3.1 mL) and potassium carbonate (2.75 g) in DMF (50 mL) was heated at 70° C. for 4 h. The reaction mixture was concentrated in vacuo. The residue was preabsorbed on silica gel and purified by chromatography (silica gel, 0 to 1% [9:1 MeOH/20M aqueous ammonia] in DCM to give the title compound as a white solid (1.1 g); ESMS m/z 290.3 [M+H]⁺.

### b) 9-(2-tert-Butoxycarbonylamino-ethylsulfanyl)-5-methyl-1-oxo-6,7-dihydro-1H,5H-pyrido[3,2,1-ij]quinoline-2-carboxylic acid ethyl ester

A suspension of Intermediate 16a (1.1 g), tert-butyl N-(2-mercaptoethyl)-carbamate (2.89 mL) and potassium carbonate (2.36 g) in dimethylsulfoxide (60 mL) was heated at 100° C. for 24 h. The reaction mixture was cooled, filtrated then partitioned between water and DCM. The combined organic extracts were dried (MgSO₄), concentrated in vacuo and the residue was purified by chromatography (silica gel, 0 to 3% [9:1 MeOH/20M aqueous ammonia] in DCM to give the title compound as a beige solid (1.7 g) contaminated with tert-butyl N-(2-mercaptoethyl)-carbamate (0.8 g); ESMS m/z 447.3 [M+H]⁺.

### c) 9-(2-Amino-ethylsulfanyl)-5-methyl-1-oxo-6,7-dihydro-1H,5H-pyrido[3,2,1-ij]quinoline-2-carboxylic acid ethyl ester

A solution of Intermediate 16b (1.7 g) in DCM (100 mL) was treated with TFA (8 mL) and the reaction stirred for 1 h. The solution was evaporated and the residue was partitioned between water and DCM. The combined organic extracts were dried (MgSO₄), concentrated in vacuo to give the title compound as a beige solid (0.92 g); ESMS m/z 347.3 [M+H]⁺.

### d) 9-(2-Amino-ethylsulfanyl)-5-methyl-1-oxo-6,7-dihydro-1H,5H-pyrido[3,2,1-ij]quinoline-2-carboxylic acid trifluoroacetate salt

A suspension of Intermediate 16c (0.92 g) in 1,4-dioxan (20 mL) was ultrasonicated then treated with 0.2N sodium hydroxide (1.46 mL). The reaction was stirred for 17 h. Solid carbon dioxide was added and the reaction mixture was concentrated. The residue was treated with DCM/TFA (3 mL/3 mL) and purified by reverse phase HPLC to give the title compound as a white solid (0.615 g); ESMS m/z 319.3 [M+H]⁺.

### Intermediate 17: 6-(2-Aminoethylsufanyl)-1-dimethylamino-4-oxo-1,4-dihydro-[1,8]-naphthyridine-3-carboxylic acid trifluoroacetate salt

### a) [1-(5-Bromo-2-chloro-3-pyridin-3-yl)methanoyl]dimethylaminoacrylic acid ethyl ester

A stirred suspension of 2-chloro-5-bromo pyridine-3-carboxylic acid (5 g) in DCM (50 mL) at 20° C. was treated with oxalyl chloride (2.8 mL) and DMF (1 drop). After 1 h the clear solution was evaporated and re-evaporated from toluene (2×). The acid chloride was re-dissolved in toluene (80 mL) and treated with triethylamine (4.7 mL) and ethyl 3-dimethylaminopropenoate (3.94 g). After stirring for 1 h at 90° C. the mixture cooled and poured onto ice. Saturated aqueous sodium hydrogen carbonate (50 mL) was added and the organic layer washed with water and brine, dried (MgSO₄) evaporated and the residue purified by chromatography (silica gel, 50-100% Et₂O in petroleum ether [b.p. 40-60° C.]) to give the title compound as a colourless gum, (6.45 g); ESMS m/z 361.0, 363.0, 365.0 [M+H]⁺, 315.0, 317.0, 319.0 [M-OEt]⁺.

### b) [1-(5-Bromo-2-chloro-3-pyridin-3-yl)methanoyl]2,2-dimethylhydrazino acrylic acid ethyl ester

A stirred suspension of Intermediate 17a (6.45 g) in ethanol (50 mL) was treated with 1,1-dimethylhydrazine (1.29 mL). After standing overnight the colourless solid was collected by filtration. A second crop was obtained by concentrating the mother liquors to give the title compound as a white solid (6.36 g); ESMS m/z 376.0, 378.0, 380.0 [M+H]⁺.

### c) 6-Bromo-1-dimethylamino-4-oxo-1,4-dihydro-[1,8]-naphthyridine-3-carboxylic acid ethyl ester

A mixture of Intermediate 17b (3.65 g) and potassium carbonate (3.5 g) in DMF (50 mL) was stirred at 60° C. for 1 h and then cooled. The mixture was poured into water, the solid filtered off then washed with water and dried to give the title compound as a white solid, (5.36 g); ESMS m/z 340.0, 342.0 [M+H]⁺.

### d) 6-(2-tert-Butylcarbonylaminoethylsufanyl)-1-dimethylamino-4-oxo-1,4-dihydro-[1,8]-naphthyridine-3-carboxylic acid ethyl ester

Intermediate 17c (1.5 g) was suspended in DMSO (20 mL) treated with potassium carbonate (1.12 g) and tert-butyl N-(2-mercaptoethyl)-carbamate (1.06 g). The mixture was stirred at 55° C. for 2.25 h and then cooled, poured into water and extracted with EtOAc. The EtOAc extracts were dried (MgSO₄) evaporated, and the residue triturated with diethyl ether/petroleum ether [b.p. 40-60° C.] (1:1, 20 mL) to give the title compound as a yellow solid (1.46 g); ESMS m/z 437.2 [M+H]⁺.

### e) 6-(2-tert-Butylcarbonylaminoethylsufanyl)-1-dimethylamino-4-oxo-1,4-dihydro-[1,8]-naphthyridine-3-carboxylic acid

Intermediate 17d (1.46 g) in THF (30 mL) and water (10 mL) was treated with 2M NaOH (2 mL) and the mixture stirred under argon for 17 h. Solid carbon dioxide was added to precipitate a yellow solid. This was filtered, washed with water and dried to give the title compound as a pale yellow solid (0.954 g); ESMS m/z 409.2 [M+H]⁺.

### f) 6-(2-Aminoethylsufanyl)-1-dimethylamino-oxo-1,4-dihydro-[1,8]-naphthyridine-3-carboxylic acid trifluoroacetate salt

Intermediate 17e (0.954 g) in DCM (10 mL) was treated with TFA (5 mL). After 15 min toluene (15 mL) was added, and the mixture evaporated to dryness to give a yellow gum. Addition of Et₂O and sonication gave the title compound as a white solid, (1.01 g); δ_{H} (250 MHz; DMSO-d₆) 14.4 (1H, bs), 9.1 (1H, d), 9.0 (1H, s), 8.7 (1H, d), 8.0 (3H, bs) 3.4 (2H, t), 3.2 (6H, s), 3.0 (2H, bm).

### Intermediate 18: 4″-O-Vinvisulfonyl-ervthromycin A-(9E)-Oxime

### a) 4″-O-Vinylsulfonyl-2′-O-acetyl-erythromycin A-(9E)-O-acetyl-oxime

To a stirred mixture of 2′-O-acetyl-erythromycin A-(9E)-O-acetyl-oxime (12.36 g) in toluene (150 mL) were added triethylamine (6.2 mL) and then dropwise 2-chloro-1-ethanesulfonyl chloride (2.4 mL) at 20° C. under argon. After 16 hours, additional triethylamine (3.1 mL) and 2-chloro-1-ethanesulfonyl chloride (2.1 mL) were added. The mixture was stirred for 30 minutes then concentrated in vacuo and the residue purified by flash chromatography (silica gel, 0-10% MeOH in DCM) to give the title compound as a brown solid (8.86 g); ESMS m/z 923.7 [M+H]⁺.

### b) 4″-O-Vinylsulfonyl-erythromycin A-(9E)-oxime

A solution of Intermediate 18a (8.54 g) in MeOH (200 mL) was stirred at 55° C. for 24 h. The mixture was concentrated in vacuo and the residue purified by flash chromatography (silica gel, 0-20% MeOH in DCM) to give the title compound as a white solid (5.66 g); ESMS m/z 839.7 [M+H]⁺.

### Intermediate 19: 6-(3-Aminopropyl)-1-(2-fluoroethyl)-4-oxo-1,4-dihydro-quinoline-3-carboxylic acid trifluoroacetate salt

### a) 6-(3-tert-Butoxycarbonylaminopropyl)-1 (2-fluoroethyl)-4-oxo-1,4-dihydro-quinoline-3-carboxylic acid ethyl ester

To a stirred solution of Intermediate 8b (1.0 g) in DMF (10 mL) was added sodium carbonate (0.42 g) and 1-fluoro-2-iodoethane (0.7 g), and the mixture heated to 60° C. After 18 h the mixture was concentrated in vacuo to give a residue which was taken up in water and extracted with EtOAc. The organic extracts were combined, dried (MgSO₄), filtered, and concentrated in vacuo to give a residue which was purified by flash chromatography (silica gel, 0-5% methanolic ammonia [2M] in DCM) to give the title compound as a white solid (0.91 g); ESMS m/z 421.3 [M+H]⁺.

### b) 6-(3-tert-Butoxycarbonylaminopropyl)-1-(2-fluoroethyl)-4-oxo-1,4-dihydro-quinoline-3-carboxylic acid

Intermediate 19a (0.9 g) was dissolved in THF (15 mL) and treated with 0.2N aqueous sodium hydroxide (13 mL). The solution was stirred for 25 h then concentrated in vacuo to give a residue which was taken up in water and treated with excess solid carbon dioxide. The resulting precipitate was removed by filtration and dried in vacuo to give the title compound as a white solid (0.8 g); ESMS m/z 393.2 [M+H]⁺.

### c) 6-(3-Aminopropyl)-1-(2-fluoroethyl)-4-oxo-1,4-dihydro-quinoline-3-carboxylic acid trifluoroacetate salt

Intermediate 19b (0.79 g) was dissolved in DCM (12 mL) and treated with TFA (4 mL). After stirring for 40 min the mixture was concentrated in vacuo. Toluene and DCM were added to the residue and the mixture concentrated in vacuo to give the title compound as a white solid; ESMS m/z 293.2 [M+H]⁺.

Intermediate 20: 4″-O-Vinylsulfonyl-azithromycin-11,12-carbonate

### a) 2′-O-Acetyl-4″-O-vinylsulfonyl-azithromycin-11,12-carbonate

To a stirred solution of 2′-O-acetyl-azithromycin-11,12-carbonate (2.2 g) in toluene (15 mL) was added triethylamine (1.31 mL), followed by 2-chloro-1-ethanesulfonyl chloride (0.56 mL). After 1 h the mixture was treated with a saturated sodium bicarbonate solution, then extracted with EtOAc. The organic extracts were combined, dried (Na₂SO₄), filtered, and concentrated in vacuo to give a solid which was purified by flash chromatography (silica gel, 0-6% methanolic ammonia [2M] in DCM) to give the title compound as a white solid (0.36 g); ESMS m/z 907.8 [M+H]⁺.

### b) 4″-O-vinylsulfonyl-azithromycin-11,12-carbonate

To Intermediate 20a (0.36 g) was added MeOH (25 mL) and the mixture heated to 50° C. After 23 h the solvent was removed in vacuo to give a solid which was purified by flash chromatography (silica gel, 0-5% methanolic ammonia [2M] in DCM) to give the title compound as a white solid (0.22 g); ESMS m/z 865.6 [M+H]⁺.

### Intermediate 21: 6-(3-Hydroxypropyl)-1,4-dihydro-1-ethyl-4-oxo-quinoline-3-carboxylic acid

### a) 1,4-Dihydro-1 ethyl-6-iodo-4-oxo-quinoline-3-carboxylic acid ethyl ester

A mixture of 1,4-dihydro-6-iodo-4-oxo-quinoline-3-carboxylic acid (J. Ellis, E. Gellert, J. Robson, Aust J. Chem., 1973, 26, 907) (3.15 g), potassium carbonate (6.9 g) and iodoethane (15.6 g) in dry DMF was heated at 70° C. with vigorous stirring. After 16 h the mixture was cooled and diluted with EtOAc. The resultant mixture was washed with water and the organic phase separated, dried and evaporated to yield the title compound as pale yellow solid, ¹H NMR δ_{H} (CDCl₃) 1.41 (3H, t, J=7.1 Hz), 1.54 (3H, t, J=7.3 Hz), 4.23 (2H, q, J=7.2 Hz), 4.40 (2H, q, J=7.1 Hz), 7.20 (1H, d, J=8.9 Hz), 7.95 (1H, dd, J=2.1 & 8.9 Hz), 8.48 (1H, s), 8.86 (1H, d, J=2.1 Hz).

### b) 6-(3-Hydroxyprop-1-ynyl)-1,4-dihydro-1-ethyl-4-oxo-quinoline-3-carboxylic acid ethyl ester

Intermediate 21a (11.57 g) and copper (I) iodide (0.2 g) were suspended in dry MeCN (300 mL) and triethylamine (180 mL). The light green suspension was heated to 50° C. whilst argon was bubbled through. After 20 min, dichlorobis(triphenylphosphine)palladium (II) (0.3 g) and propargyl alcohol (4.01 g) were added and the brown suspension was heated under argon at 50° C. for 16 h. The crude product was purified by chromatography on silica gel eluting with DCM in hexane followed by a gradient of MeOH in DCM (0-5%). Product containing fractions were evaporated to dryness and the residue dissolved in chloroform and filtered. The filtrate was evaporated to dryness to yield the title compound as a beige solid, (10.92 g); ESMS m/z 300.2 [M+H]⁺.

### c) 6-(3-Hydroxypropyl)-1,4-dihydro-1-ethyl-4-oxo-quinoline-3-carboxylic acid ethyl ester

Intermediate 21b (10.9 g) in ethanol (300 mL) was hydrogenated at 20° C. at atmospheric pressure over 10% Pd/C (2 g) for 16 h. The catalyst was removed by filtration, a further 2 g of fresh catalyst added and the hydrogenation continued for 3 d. The catalyst was removed by filtration and the crude product purified by chromatography on silica gel eluting with a gradient of MeOH in DCM (0-10%) to yield the impure title compound, (7.27 g). A portion of this (2 g) was dissolved in EtOAc (100 mL) and washed with water (50 mL) and brine (50 mL). The organic layer was dried (MgSO₄) and evaporated to give the pure title compound as a white solid (1.4 g); ESMS m/z 326.2 [M+Na]⁺, 258.3 [M-OEt]⁺.

### d) 1-Ethyl-6-(3-hydroxypropyl)-4-oxo-1,4-dihydro-quinoline-3-carboxylic acid

A solution of Intermediate 21c (0.204 g) in MeOH (2 mL) and 1N aqueous sodium hydroxide (1 mL) was heated at 50° C. for 2 h. The solution was evaporated to low volume and then acidified. The solid was filtered off, washed with water and dried to give the title compound as a white solid (0.162 g); APCI m/z 276.1 [M+H]⁺.

### Intermediate 22: 4″-O-Vinylsulfonyl-erythromycin A-(9E)-O-methoxymethyloxime

### a) 2′-O-Acetyl-erythromycin A-(9E)-O-methoxymethyloxime

To a stirred mixture of erythromycin A-(9E)-O-methoxymethyloxime (0.48 g) and sodium hydrogen carbonate (0.056) in DCM (10 mL) at 20° C. was added acetic anhydride (0.068 g). After 16 h the mixture was concentrated and the residue purified by flash chromatography (silica gel, 0-10% MeOH in DCM) to give the title compound as a white solid (0.44 g); ESMS m/z 835.8 [M+H]⁺.

### b) 4″-O-Vinylsulfonyl-2′-O-acetyl-erythromycin A-(9E)-O-methoxymethyloxime

To a stirred mixture of Intermediate 22a (0.43 g) in toluene (15 mL) were added triethylamine (0.22 mL) and then dropwise 2-chloro-1-ethane sulfonyl chloride (0.081 mL) at 20° C. under argon. After 16 hours, additional triethylamine (0.15 mL) and 2-chloro-1-ethane sulfonyl chloride (0.054 mL) were added. The mixture was stirred for 2 h then concentrated in vacuo and the residue purified by flash chromatography (silica gel, 0-10% MeOH in DCM) to give the title compound as a white solid (0.34 g); ESMS m/z 925.7 [M+H]⁺.

### c) 4″-O-Vinylsulfonyl-erythromycin A-(9E)-O-methoxymethyloxime

A solution of Intermediate 22b (0.32 g) In MeOH (15 mL) was stirred at 55° C. for 16 h. The mixture was concentrated in vacuo to give the title compound as a white solid (0.29 g); ESMS m/z 883.8 [M+H]⁺.

### Intermediate 23: 4″-O-Vinylsulfonyl-erythromycin A-(9E)-oxime-11,12-carbonate

### (a) 4″-O-Vinylsulfonyl-2′-O-acetyl-erythromycin A-(9E)-O-acetyl-oxime

To a stirred mixture of 2′-G-acetyl-erythromycin A-(9E)O-acetyl-oxime (12.36 g) in toluene (150 mL) were added triethylamine (6.2 mL) and then dropwise 2-chloro-1-ethanesulfonyl chloride (2.4 mL) at 20° C. under argon. After 16 h, additional triethylamine (3.1 mL) and 2-chloro-1-ethane sulphonyl chloride (2.1 mL) were added. The mixture was stirred for 30 min then concentrated in vacuo and the residue purified by flash chromatography (silica gel, 0-10% MeOH in DCM) to give the title compound as a brown solid (8.86 g); ESMS m/z 923.7 [M+H]⁺.

### (b) 4″-O-Vinylsulfonyl-2′-O-acetyl-erythromycin A-(9E)-O-acetyl-oxime-11,12-carbonate

To an ice cooled solution of Intermediate 23a (1.7 g) and pyridine (1.49 mL) in DCM (15 mL) was added triphosgene (0.27 g). The mixture was stirred for 95 min then concentrated in vacuo to give a residue which was taken up in EtOAc and washed successively with a saturated sodium hydrogen carbonate solution then brine. The organic layer was dried (Na₂SO₄), filtered then concentrated in vacuo to give the title compound as a cream solid (1.93 g) which was used in the next step without further purification; ESMS m/z 949.7 [M+H]⁺.

### (c) 4″-O-Vinylsulfonyl-erythromycin A-(9E)-oxime-11,12-carbonate

A solution of Intermediate 23b (1.93 g) in MeOH (30 mL) was stirred at ca. 20° C. for 14 h, then 50° C. for 5 hours. The mixture was then concentrated in vacuo and the residue purified by flash chromatography (silica gel, 0-7% methanolic ammonia [2M] in DCM) to give the title compound as a white solid (0.81 g); ESMS m/z 865.8 [M+H]⁺.

### Intermediate 24: 1-Ethyl-4-oxo-6-(3-[(3R)-3-pyrrolidinyloxy]propyl-1,4-dihydro-3-quinolinecarboxylic acid trifluoroacetate salt

### a) 1,1-Dimethylethyl (3R)-3-hydroxy-1-pyrrolidinecarboxylate

To a solution of (R)-3-hydroxypyrrolidine (5 g) dissolved in DCM (50 mL) was added bis(1,1-dimethylethyl) dicarbonate (12.5 g). After stirring for 16 h the mixture was quenched with water (50 mL) and the organic layer separated, dried and evaporated to yield a colourless gum. Crystallisation from hexane gave the title compound as a white solid (4.4 g); [α]_{D} (c=1, CHCl₃)-21.8°, δ_{H} (400 MHz; CDCl₃) 1.46 (9H, s), 2.02-1.84 (2H, m), 2.31-2.25 (1H, m), 3.51-3.31 (4H, m), 4.45-4.42 (1H, m).

### b) 1,1-Dimethylethyl (3R)-3-(2-propyn-1-yloxy)-1-pyrrolidinecarboxylate

Intermediate 24a (4 g) in THF (20 mL) was added dropwise to a suspension of sodium hydride (0.86 g) in THF (15 mL). After stirring for 2 h an 80% solution of 3-bromo-1-propyne in toluene (2.7 mL) was added and the resultant mixture heated under reflux for 14 h. The mixture was cooled and partitioned between Et₂O (100 mL) and water (50 mL). The organic phase was separated, dried and evaporated to yield the crude product. Chromatography over silica gel eluting with hexane containing an increasing concentration of the Et₂O (0-60%) gave the title compound as a yellow syrup (4.17 g); δ_{H} (400 MHz; CDCl₃) 1.45 (9H, m), 2.02-1.95 (2H, m), 2.44 (1H, t), 3.47-3.39 (4H, m), 4.17 (2H, d), 4.27 (1H, s).

### c) Ethyl 6-{3-[((3R)-1-{[(1,1-dimethylethyl)oxy]carbonyl}-3-pyrrolidinyl)oxy]-1-propyn-1-yl}-1-ethyl-4-oxo-1,4-dihydro-3-quinolinecarboxylate

Through a solution of Intermediate 6a (2.55 g) and copper (I) iodide (0.03 g) dissolved in MeCN (100 mL) and triethylamine (70 mL) at 40° C. was bubbled argon. After 0.5 h the mixture was cooled to ca. 20° C. and Intermediate 24b (3.3 g) and dichlorobis(triphenylphosphine)palladium (II) (0.04 g) were added. The mixture was stirred for a further 4 h, after which time the MeCN was evaporated and the crude product purified by chromatography (silica gel, 0-5% MeOH in DCM) to yield the title compound as a yellow solid (3.03 g); ESMS m/z 469.2 [M+H]⁺.

### d) Ethyl 6-{3-[((3R)-1-([(1,1-dimethylethyl)oxy]carbonyl)-3-pyrrolidinyl)oxy]propyl}-1-ethyl-4-oxo-1,4-dihydro-3-quinolinecarboxylate

Intermediate 24c (3.03 g) in DCM (100 mL) was hydrogenated over 10% palladium on charcoal (0.50 g) for 14 h. The resultant mixture was filtered and the solvent evaporated to give the title compound as a yellow syrup (3.0 g); δ_{H} (400 MHz; CDCl₃) 1.42 (3H, t), 1.47 (9H, s), 1.55 (3H, t), 1.98-1.92 (4H, m), 2.83 (2H, t), 3.60-3.32 (6H, complex m), 3.98 (1H, m), 4.25 (2H, q), 4.40 (2H, q), 7.39 (1H, d), 7.52 (1H, d), 8.36 (1H, s), 8.49 (1H, s).

### e) 6-{3-[((3R)-1-{[(1,1-Dimethylethyl)oxy]carbonyl}-3-pyrrolidinyl)oxy]propyl}-1-ethyl-4-oxo-1,4-dihydro-3-quinolinecarboxylic acid

To a solution of Intermediate 24d (1.5 g) in THF (20 mL) was added 2M sodium hydroxide (3.1 mL). After stirring for 48 h the mixture was saturated with carbon dioxide and the solvent evaporated. The residue was treated with MeOH (40 mL), filtered and the solvent evaporated to yield the title compound as a pale yellow gum (1.15 g); ESMS m/z 445.2 [M+H]⁺.

### f) 1-Ethyl-4-oxo-6-{3-[(3R)-3-pyrrolldinyloxy]propyl}-1,4-dihydro-3-quinolinecarboxylic acid trifluoroacetate salt

To Intermediate 24e (1.15 g was added TFA (5 mL) in DCM (5 mL). After 2 h the solvent was evaporated to yield the title compound as a yellow oil (1.83 g); ESMS m/z 345.2 [M+H]⁺.

### Intermediate 25: 7-(2-Aminoethoxy)-1-dimethylamino-4-oxo-1,4-dihydroquinoline-3-carboxylic acid hydrochloride salt

### a) 4-Acetoxy-2-fluorobenzoic acid

A stirred mixture of 2-fluoro-4-hydroxybenzoic acid (5 g) (G. W. Gray et at, Mol. Cryst. Liq. Cryst., 67, 1981, 1-24), 4-dimethylaminopyridine (0.01 g) and triethylamine (11 mL) in DCM (100 mL) was treated with acetic anhydride (6.35 mL). After 2 h the solution was evaporated and the residue redissolved in EtOAc, washed with 5% citric acid, water (3×), dried (Na₂SO₄) and evaporated to give the title compound as a white solid (4.92 g); APCI m/z 199.1 [M+H]⁺.

### b) Ethyl 3-dimethylamino-2-(2-fluoro-4-acetoxybenzyl)-2-propenoate

A stirred solution of Intermediate 25a (4.91 g) in DCM (80 mL) at 20° C. was treated with oxalyl chloride (3.25 mL) and DMF (2 drops). After 2 h the clear solution was evaporated and re-evaporated from DCM (2×). The acid chloride was re-dissolved in toluene (100 mL) and treated with triethylamine (5.17 mL) and ethyl 3-dimethylaminopropenoate (4.13 g). After stirring for 2 h at 90° C. the mixture was filtered and the solution flash chromatographed (silica gel 40 to 100% EtOAc in petroleum ether [b.p. 40-60° C.]) to give the title compound as a yellow gum (4.3 g); APCI m/z 324.0 [M+H]⁺.

### c) Ethyl 1-dimethylamino-7-hydroxy-4-oxo-1,4-dihydro-quinoline-3-carboxylate

A solution of Intermediate 25b (1.77 g) in ethanol (20 mL) at 20° C. was treated with 1,1-dimethylhydrazine (2.08 mL) and stood for 2 h. The solution was evaporated and the residue redissolved in DMF (20 mL), treated with potassium carbonate (1.51 g) and stirred at 100° C. for 1 h. The mixture was cooled, filtered, evaporated to low volume and poured into 5% citric acid. The title compound was filtered off as a white solid, washed with water and dried (1.09 g), APCI m/z 277.0 [M+H]⁺.

### d) Ethyl 7-(2-tert-butoxycarbonylaminoethoxy)-1-dimethylamino-4-oxo-1,4-dihydro-quinoline-3-carboxylate

A stirred mixture of Intermediate 25c (1.09 g), tert-butyl 2-hydroxyethylcarbamate (0.67 mL) and triphenylphosphine (1.35 g) in dry THF (20 mL) under argon was treated with diisopropyl azodicarboxylate (1 mL) and stirred overnight. The solution was evaporated and the residue redissolved in EtOAc, washed with 5% sodium carbonate solution (2×) and water (2×), and then dried (Na₂SO₄), evaporated and flash chromatographed (silica gel 30 to 50% EtOAc in DCM then 5% MeOH in DCM) to give the title compound as a white solid (1.5 g); APCI m/z 420.3 [M+H]⁺.

### e) 7-(2-tert-Butoxycarbonylaminoethoxy)-1-dimethylamino-4-oxo-1,4-dihydro-quinoline-3-carboxylic acid

Intermediate 25d (1.5 g) was suspended in MeOH (10 mL) and treated with 1N aqueous sodium hydroxide (5.4 mL). The mixture was stirred overnight. The solution was evaporated to low volume, acidified with 5% citric acid and extracted with DCM (2×). The combined extracts were washed with water (2×), dried (Na₂SO₄) and evaporated to give the title compound as a white solid (1.27 g); APCI m/z 392.2 [M+H]⁺.

### f) 7-(2-Aminoethoxy)-1-dimethylamino-4-oxo-1,4-dihydroquinoline-3-carboxylic acid hydrochloride salt

A solution of Intermediate 25e (1.27 g) in DCM (10 mL) was treated with 4M HCl in 1,4-dioxan (5 mL). After 2 h the solid was filtered off, washed with acetone and dried to give the title compound as a white solid (1 g); APCI m/z 292.2 [M+H]⁺.

### Intermediate 26: 4″-O-Vinylsulfonyl-azithromycin

### a) 2′-O-Acetylazithromycin-11,12-methyl boronate

To a stirred solution of 2′-O-acetylazithromycin (1.51 g) in toluene. (20 mL) was added trimethylboroxin (0.087 mL), and the mixture heated to reflux. After 50 min the mixture was allowed to cool, then concentrated in vacuo to give the title compound as a white solid (1.56 g); ESMS m/z 815.8 [M+H]⁺.

### b) 2′-O-Acetyl-4″-O-vinylsulfonyl-azithromycin-11,12-methyl boronate

To a stirred solution of Intermediate 26a (4.3 g) in toluene (100 mL) at −78° C. was added triethylamine (4.42 mL), followed by 2-chloro-1-ethanesulfonyl chloride (1.66 mL). After 6 h the mixture was treated with a saturated sodium bicarbonate solution. The mixture was adjusted to pH 12 by addition of sodium carbonate, then extracted with EtOAc. The organic extracts were combined, dried (Na₂SO₄), filtered, and concentrated in vacuo to give the crude title compound as a white solid (4.86 g) which was used in the next step without further purification; ESMS m/z 905.9 [M+H]⁺.

### c) 4″-O-vinylsulfonyl-azithromycin

A stirred solution of crude Intermediate 26b (4.86 g) in MeOH (100 mL) was heated to reflux. After 39 h further MeOH (100 mL) was added and the mixture heated for a further 1.5 h. The solvent was then removed in vacuo to give a solid which was purified by flash chromatography (silica gel, 0-25% MeOH in DCM) to give the title compound as a white solid (0.59 g); ESMS m/z 839.9 [M+H]⁺.

### Intermediate 27: 4″-O-Vinylsulfonyl-erythromycin A-11,12-carbonate

### (a) 4″-O-Vinylsulfonyl-2′-O-acetyl-erythromycin A-11,12-carbonate

To a stirred solution of 2′-O-acetyl-erythromycin A-11,12-carbonate (4.55 g) in toluene (20 mL) was added triethylamine (1.46 mL), followed by 2-chloro-1-ethanesulfonyl chloride (1.18 mL). After 2.5 h further triethylamine (0.71 mL) and 2-chloro-1-ethanesulfonyl chloride (0.30 mL) were added. Stirring was continued for a further 72 h and the mixture then treated with a saturated sodium bicarbonate solution and extracted with DCM. The organic extracts were combined, dried (Na₂SO₄), filtered, and concentrated in vacuo to give a residue which was purified by flash chromatography (silica gel, 0-8% MeOH in DCM) to give the title compound as a white solid (3.32 g); ESMS m/z 892.8 [M+H]⁺.

### (b) 4″-O-Vinylsulfonyl-erythromycin A-11,12-carbonate

A solution of Intermediate 27a (2.89 g) in MeOH (150 mL) was stirred at 50° C. for 6.5 h. The mixture was then concentrated in vacuo and the residue purified by flash chromatography (silica gel, 0-10% MeOH in DCM) to give the title compound as a white solid (1.73 g); ESMS m/z 850.8 [M+H]⁺.

### Intermediate 28: 10-(3-Aminopropyl)-8-oxo-3,4-dihydro-1H,8H-[1,4]oxazepino[6,5,4-ij]quinoline-7-carboxylic acid

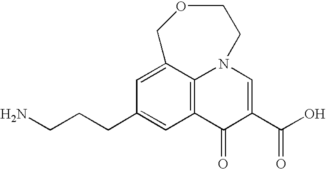

### a) (2-Amino-5-bromophenyl)methanol

1.0M Lithium aluminium hydride in THF (175 mL) was added to a solution of methyl 2-amino-5-bromobenzoate (40.37 g) in THF (400 mL) by cannula at 0° C. under argon. Stirring was continued for 1 hour after addition. Water (6.4 mL), 2M sodium hydroxide (6.4 mL) and water (12.8 mL) were added sequentially and the resultant mixture stirred at 20° C. for 30 minutes before filtration through celite. The solution was evaporated and the residue crystallised from EtOAc/hexane to give the title compound as a white solid (18.5 g); ESMS m/z 202.1 [M+H]⁺.

### b) N-[4-Bromo-2-(hydroxymethyl)phenyl]-2-chloroacetamide

To a suspension of Intermediate 28a (2.48 g) in dry Et₂O (70 mL) at 0° C. was added dropwise a solution of chloroacetyl chloride (1.39 g) in dry toluene (25 mL). To the white suspension was added a solution of triethylamine (5.1 mL) in dry toluene (25 mL). After 1 h the reaction mixture was treated with water and chloroform. The organic phase was dried, filtered, and concentrated in vacuo to give a brown oil (4.45 g) containing the title compound; ESMS m/z 278.0/280.0 [M+H]⁺ and the bis acetylated compound {5-bromo-2-[(chloroacetyl)amino]phenyl}methyl chloroacetate; ESMS m/z 352.0/354.0/356.0 [M+H]⁺.

### c) 7-Bromo-1,5-dihydro-4,1-benzoxazepin-2(3M-one

A fresh solution of sodium ethoxide in ethanol was prepared from sodium (0.5 g) and dry ethanol (60 mL). This solution was added to Intermediate 28b (4.25 g) containing a mixture of mono N-acetylated (6 mmol) and bis acetylated (7.3 mmol) material). The suspension was heated to 80° C. for 1.5 h then allowed to cool down. Filtration of the solid followed by trituration with hexane provided the title compound as a beige solid (3 g); ESMS m/z 242.1/244.1 [M+H]⁺.

### d) 7-Bromo-1,2,3,5-tetrahydro-4,1-benzoxazepine

To a suspension of Intermediate 28c (2.5 g)) in dry Et₂O (60 mL) was added a solution of lithium aluminium hydride in THF (1M, 2.58 mL). After 1.25 h an additional amount of lithium aluminium hydride in THF (1M, 10 mL) was added and the reaction mixture stirred for one hour. After the addition of water (6 mL), the mixture was filtered and the solid washed with diethyl ether. The filtrate was evaporated and re-dissolved in water. Freeze drying of this aqueous solution gave the title compound as a white solid (2 g); ESMS m/z 228.1/230.1 [M+H]⁺.

### e) Diethyl[(7-bromo-2,3-dihydro-4,1-benzoxazepin-1(5M)-yl)methylidene]propanedioate

A mixture of Intermediate 28d (2 g) and diethyl ethoxymethylenemalonate (1.77 mL) was heated at 115° C. for 5 h. The reaction mixture was allowed to cool and purified by chromatography eluting with DCM to give the crude title compound (3 g) which was used as such in the next step; ESMS m/z 398.2/400.2 [M+H]⁺.

### f) Ethyl 10-bromo-8-oxo-3,4-dihydro-1H,8H-[1,4]oxazepino[6,5,4-ij]quinoline-7-carboxylate

A mixture of Intermediate 28e (2 g) in an excess of polyphosphoric acid was heated to 100° C. for 1 h. Ice was added followed by ultrasonication and DCM extraction. The organic phase was dried then concentrated during this process some of the title compound precipitated out. Filtration provided a white solid (0.96 g); ESMS m/z 352.2/354.2 [M+H]⁺. Further material (0.3 g) was obtained from the dichlormethane residues by chromatography (slica 0 to 3% MeOH in DCM).

### g) Sodium 10-Bromo-8-oxo-3,4-dihydro-1H,8H-[1,4]oxazepino[8,5,4-ij]quinoline-7-carboxylate

A solution of Intermediate 28f (1.17 g) in 1,4-dioxan (40 mL) was treated with 2M sodium hydroxide (1.83 mL). After stirring at 50° C. for 17 h the temperature was increased to 60° C. for 2.5 h then to 70° C. for 3 h. The mixture was treated with solid carbon dioxide and concentrated to give the title compound as a white solid (1.3 g); ESMS m/z 324.1/326.1 [M+H]⁺.

### h) 10-[3-({[(1,1-Dimethylethyl)oxy]carbonyl}amino)-1-propyn-1-yl]-8-oxo-3,4-dihydro-1H,8H-[1,4]oxazepino[6,5,4-ij]quinoline-7-carboxylic acid

A suspension of Intermediate 28 g (1.07 g), copper (I) iodide (0.063 g) and triethylamine (13 mL) in dry DMF (22 mL) was ultrasonicated whilst argon was bubbled through. After 0.5 h, dichlorobis(triphenylphosphine)palladium (II) (0.07 g) and N-tert-butoxycarbonylpropargylamine (0.82 g) were added and the mixture was heated at 100° C. for 1.5 h. The reaction mixture was cooled, concentrated in vacuo and purified by chromatography (silica gel, 0 to 10% [9:1 MeOH/0.880 ammonia] in DCM to give after trituration with Et₂O the title compound as a white solid (0.8 g); ESMS m/z 399.3 [M+H]⁺.

### i) 10-[3-({[(1,1-Dimethylethyl)oxy]carbonyl}amino)propyl]-8-oxo-3,4-dihydro-1H,8H-[1,4]oxazepino[6,5,4-ij]quinoline-7-carboxylic acid

A solution of Intermediate 28 h (0.8 g) in DMF (20 mL) was treated with 10% palladium on carbon (0.6 g) and hydrogenated at room temperature and atmospheric pressure for 17 h. The reaction mixture was filtered through celite with some charcoal and concentrated to give the title compound as a white solid (0.5 g); ESMS m/z 403.3 [M+H]⁺.

### j) 10-(3-Aminopropyl)-8-oxo-3,4-dihydro-1H,8H-[1,4]oxazepino[6,5,4-ij]quinoline-7-carboxylic acid

A solution of Intermediate 281 (0.5 g) in DCM (20 mL) was treated with TFA (10 mL) and the reaction stirred at room temperature for 20 min. The solution was evaporated to dryness and triturated with Et₂O to give the title compound as a white solid (0.53 g); ESMS m/z 303.3 [M+H]⁺.

### Intermediate 29: 7-(3-Aminopropoxy)-1-dimethylamino-4-oxo-1,4-dihydroquinoline-3-carboxylic acid hydrochloride

a) Ethyl 7-(3-tert-butoxycarbonylaminopropoxy)-1-dimethylamino-4-oxo-1,4-dihydro-quinoline-3-carboxylate

A stirred mixture of Intermediate 25c (1.07 g), tert-butyl 3-hydroxypropylcarbamate (0.75 g) and triphenylphosphine (1.32 g) in dry THF (20 mL) under argon was treated with diisopropyl azodicarboxylate (0.98 mL) and stirred overnight. The solution was evaporated and the residue redissolved in EtOAc, washed with 5% sodium carbonate solution (2×) and water (2×), dried (Na₂SO₄), evaporated and flash chromatographed (silica gel, 30 to 50% EtOAc in DCM then 5% MeOH in DCM) to give the title compound as a white solid (1.54 g); APCI m/z 434.1 [M+H]⁺.

### b) 7-(3-tert-Butoxycarbonylaminopropoxy)-1-dimethylamino-4-oxo-1,4-dihydro-quinoline-3-carboxylic acid

Intermediate 29a (1.53 g) was suspended in MeOH (10 mL) and treated with 1M aqueous sodium hydroxide (5.3 mL). The mixture was stirred overnight. The solution was evaporated to low volume and acidified with 5% citric acid. The solid was filtered off washed with water and dried to give the title compound as a white solid (1.4 g); APCI m/z 406.1 [M+H]⁺.

### c) 7-(3-Aminopropoxy)-1-dimethylamino-4-oxo-1,4-dihydro-quinoline-3-carboxylic acid hydrochloride

A solution of Intermediate 29b (1.4 g) in DCM (10 mL) was treated with 4M HCl in 1,4-dioxan (5 mL). After 2 h the solid was filtered off, washed with acetone and dried to give the title compound as a white solid (1.16 g); APCI m/z 306.1 [M+H]⁺.

### Intermediate 30: 1-Dimethylamino-6-(3-hydroxypropyl)-4-oxo-1,4-dihydroquinoline-3-carboxylic acid

### a) 1-Dimethylamino-6-(3-hydroxypropyl)-4-oxo-1,4-dihydro-3-quinolinecarboxylic acid

A stirred suspension of Intermediate 4d (2 g) and copper (I) iodide (0.106 g) in triethylamine (25 mL) and MeCN (35 mL) was degassed and covered with argon. After 15 min propargyl alcohol (0.49 mL) and dichlorobis(triphenylphosphine)palladium (II) (0.123 g) were added. After 30 min the mixture was evaporated and redissolved in aqueous sodium hydroxide (0.45 g in 50 mL). The mixture was washed with Et₂O (3×), filtered and acidified with citric acid. The solid was filtered off, washed with water and dried to give the title compound as a white solid (1.6 g); APCI m/z 287.1 [M+H]⁺.

### b) 1-Dimethylamino-6-(3-hydroxypropyl)-4-oxo-1,4-dihydro-3-quinolinecarboxylic acid

A solution of Intermediate 30a (1.6 g) and sodium hydroxide (0.32 g) in MeOH (20 mL) and water (10 mL) was treated with 10% palladium on carbon (0.3 g) and hydrogenated at room temperature and atmospheric pressure for 4 h. The reaction mixture was filtered, evaporated to low volume and acidified with citric acid, the solid filtered off, washed with water and dried to give the title compound as a white solid (1.28 g); APCI m/z 291.1 [M+H]⁺.

### Intermediate 31: 1-Amino-6-(3-aminopropyl)-4-oxo-1,4-dihydroquinoline-3-carboxylic acid mono formate

### a) 2-Dimethylamino-4-hydroxy-6-iodo-1-(1,3-dioxo-1,3-dihydro-2H-isoindol-2-yl)-1,2-dihydroquinoline-3-carboxylic acid ethyl ester

A mixture of Intermediate 4b (0.78 g), N-amino phthalimide (0.194 g) and potassium carbonate (0.414 g) in DMF (10 mL) was stirred at 100° C. for 2 h and then cooled. The mixture was poured into 5% citric acid, the solid filtered off then washed with water and dried to give the title compound as a white solid (1.1 g); APCI m/z 533.8 [M+H]⁺.

### b) 1-(1,3-Dioxo-1,3-dihydro-2H-isoindol-2-yl)-6-iodo-4-oxo-1,4-dihydro-3-quinolinecarboxylic acid ethyl ester

A solution of Intermediate 31a (1.1 g) in ethanol (5 mL) at ca. 20° C. was treated with concentrated hydrochloric acid (0.2 mL) and stood overnight. The solid was filtered off, washed with ethanol and dried to give the title compound as a white solid (0.82 g); APCI m/z 488.8 [M+H]⁺.

### c) 1-(1,3-Dioxo-1,3-dihydro-2H-isoindol-2-yl)-6-(3-tert-butoxycarbonylaminopropyn-1-yl)-4-oxo-1,4-dihydro-3-quinolinecarboxylic acid ethyl ester

A stirred suspension of Intermediate 31b (1.05 g) and copper (I) iodide (0.041 g) in triethylamine (9.9 mL) and MeCN (20 mL) was degassed and covered with argon. After 15 min N-tert-butoxycarbonylpropargylamine (0.5 g) and dichlorobis(triphenylphosphine)palladium (II) (0.048 g) were added. After 30 min the mixture was evaporated and redissolved in DCM. The mixture was washed with saturated sodium hydrogen carbonate solution, water (2×), dried (Na₂SO₄) and evaporated. The residue was flash chromatographed on silica gel eluting with 10 to 40% EtOAc in DCM to give the title compound as a brown foam (0.34 g); APCI m/z 516.0 [M+H]⁺.

### d) 1-Amino 6-(3-aminopropyl)-4-oxo-1,4-dihydroquinoline-3-carboxylic acid mono formate

A solution of Intermediate 31c (0.216 g) in MeOH at ca. 20° C. was treated with 1M sodium hydroxide solution and stirred overnight. The mixture was evaporated and the residue refluxed in 5M hydrochloric acid for 4 h. The mixture was allowed to cool, the solid filtered off and the solution evaporated. The residue was purified by mass directed automatic preparative HPLC to give the title compound as a grey solid (0.019 g), APCI m/z 262.2 [M+H]⁺.

### Intermediate 32: 9-({2-[(2-Hydroxyethyl)oxy]ethyl}amino)-5-methyl-1-oxo-6,7-dihydro-1H,5H-pyrido[3,2,1-ij]quinoline-2-carboxylic acid formate salt

A suspension of flumequine (0.261 g) and 2-[(2-aminoethyl)oxy]ethanol (1.2 mL) in N-methylpyrrolidinone (1.2 mL) was microwaved at 220° C. for 1 h. The reaction mixture was purified by mass directed automatic preparative HPLC to give the title compound as a yellow solid (0.152 g); ESMS m/z 347.3 [M+H]⁺.

### Intermediate 33: 9-({2-[(2-Aminoethyl)oxy]ethyl}amino)-5-methyl-1-oxo-6,7-dihydro-1H,5H-pyrido[3,2,1-ij]quinoline-2-carboxylic acid formate salt

A suspension of flumequine (0.13 g) and {2-[(2-aminoethyl)oxy]ethyl}amine (0.25 mL) in N-methylpyrrolidinone (0.25 mL) was microwaved at 200° C. for 1 h. The reaction mixture was purified by mass directed automatic preparative HPLC to give the title compound as a yellow oil (0.144 g) containing some N-methylpyrrolidinone; ESMS m/z 346.4 [M+H]⁺.

### Intermediate 34: 9-(3-Aminopropyl)-3,3-dimethyl-7-oxo-2,3-dihydro-7H-[4]oxazino[2,3,4-ij]quinoline-6-carboxylic acid trifluoroacetate salt

### a) Ethyl (2Z)-3-dimethylamino-2-[2,3-difluoro-5-iodobenzoyl]-2-propenoate

A suspension of 2,3-difluoro-5-iodobenzoic acid (2.84 g) in DCM (50 mL) was treated with oxalyl chloride (1.3 mL). After 1.5 h the resultant solution was evaporated, re-dissolved in toluene (50 mL) and re-evaporated to yield the intermediate acid chloride. This crude material was dissolved in toluene (50 mL) and treated with triethylamine (2.1 mL) and ethyl 3-dimethylaminopropenoate (1.86 g). The mixture was stirred for 2 h at 90° C., cooled, filtered and evaporated. The crude product was purified by chromatography over silica gel eluting with 0-70% EtOAc in hexane to give the title compound as a yellow solid (3.05 g); ESMS m/z 410.2 [M+H]⁺.

### b) Ethyl (2Z)-3-[(2-hydroxy-1,1-dimethylethyl)amino]-2-[2,3-difluoro-5-iodobenzoyl]-2-propenoate

Intermediate 34a (0.408 g) was dissolved in ethanol (5 mL) and 2,2-dimethyl-2-aminoethanol (0.098 g). After stirring for 1.25 h the mixture was evaporated and the crude product purified by chromatography over silica gel eluting with 0-40% EtOAc in DCM to give the title compound as a colourless oil (0.45 g); ESMS m/z 454.2 [M+H]⁺.

### c) Ethyl 8-iodo-3,3-dimethyl-6-oxo-2,3,3a,6-tetrahydrobenzo[de]chromene-5-carboxylate

Intermediate 34b (0.405 g) was dissolved in DMF (9 mL) and treated with 1,8-diazabicyclo[5,4,0]undec-7-ene (0.27 mL). After heating at 60° C. for 16 h the mixture was cooled and the DMF evaporated and the crude product purified by chromatography over silica gel eluting with 0-40% EtOAc in DCM to give the title compound as a white solid (0.34 g); ESMS m/z 414.2 [M+H]⁺.

### d) Ethyl 8-[3-({[(1,1-dimethylethyl)oxy]carbonyl}amino)-1-propyn-1-yl]-3,3-dimethyl-6-oxo-2,3,3a,6-tetrahydrobenzo[de]chromene-5-carboxylate

Through a mixture of Intermediate 34c (0.337 g) and copper (I) iodide (0.017 g) suspended in MeCN (8 mL) at 50° C. was bubbled argon. After 20 min dichlorobis(triphenylphosphine)palladium (II) (0.017 g) and N-tert-butoxycarbonylpropargylamine (0.216 g) were added and heating continued for a further 1 h. The mixture was cooled, the MeCN evaporated and the crude product purified by chromatography over silica gel eluting with 0-2% MeOH in DCM to give the title compound as a beige solid (0.35 g); ESMS m/z 441.4 [M+H]⁺.

### e) Ethyl 8-[3-({[(1,1-dimethylethyl)oxy]carbonyl}amino)propyl]-3,3-dimethyl-6-oxo-2,3,3a,6-tetrahydrobenzo[de]chromene-5-carboxylate

A solution of Intermediate 34d (0.35 g) in DCM (20 mL) was treated with 10% palladium on carbon (0.35 g) and hydrogenated at room temperature and atmospheric pressure for 16 h. The reaction mixture was filtered and concentrated to give the title compound as a pale yellow solid (0.34 g); ESMS m/z 445.4 [M+H]⁺.

### f) 8-[3-({[(1,1-Dimethylethyl)oxy]carbonyl}amino)-1-propyl]-3,3-dimethyl-6-oxo-2,3,3a,6-tetrahydrobenzo[de]chromene-5-carboxylic acid sodium salt

A solution of Intermediate 34e (0.34 g) in THF (5 mL) was treated with 2M sodium hydroxide (0.42 mL). The reaction was stirred for 17 h at 50° C. then solid carbon dioxide was added and the reaction mixture was concentrated to give the title compound as a pale yellow solid (0.32 g); ESMS m/z 417.3 [M+H]⁺.

### g) 8-(3-Aminopropyl)-3,3-dimethyl-6-oxo-2,3,3a,6-tetrahydrobenzo[de]chromene-5-carboxylic acid trifluoroacetate salt

A solution of Intermediate 34f (0.316 g) in DCM (3 mL) was treated with TFA (3 mL) and the reaction stirred at room temperature for 20 min. The solution was evaporated to dryness to yield the title compound as a beige solid (0.24 g); ESMS m/z 317.3 [M+H]⁺.

### Intermediate 35: 6-(3-Aminopropyl)-1,4-dihydro-1-methyl-4-oxo-quinoline-3-carboxylic acid trifluoroacetate salt

### a) 6-(3-tert-Butoxycarbonylaminopropyl)-1,4-dihydro-1-methyl-4-oxo-quinoline-3-carboxylic acid ethyl ester

To a mixture of Intermediate 8b (0.496 g), and potassium carbonate (0.274 g) in DMF (5 mL) was added iodomethane (0.17 mL). After stirring for 4.5 h the mixture was diluted with EtOAc, filtered, then concentrated in vacuo. The residue was taken up in water, extracted with EtOAc, then the organic layers combined, dried (MgSO₄), filtered, and concentrated in vacuo to give the title compound as a cream solid (0.504 g); ESMS m/z 389.1 [M+H]⁺.

### b) 6-(3-tert-Butoxycarbonylaminopropyl)-1,4-dihydro-1-methyl-4-oxo-quinoline-3-carboxylic acid

A solution of Intermediate 35a (0.494 g) in THF (8 mL) was treated with 0.2 N aqueous sodium hydroxide (7.6 mL). After 29 h the mixture was concentrated in vacuo. The resulting residue was taken up in water, treated with excess solid carbon dioxide, and the precipitate which formed was filtered off and dried in vacuo to give the title compound as a cream solid (0.40 g); ESMS m/z 361.1 [M+H]⁺.

### c) 6-(3-Aminopropyl)-1,4-dihydro-1-methyl-4-oxo-quinoline-3-carboxylic acid trifluoroacetate salt

A solution of Intermediate 35b (2.82 g) in DCM (30 mL) was treated with TFA (10 mL). After 50 min the solvent was removed in vacuo, the residue taken up in toluene, the mixture concentrated in vacuo, then the residue taken up in DCM and concentrated in vacuo. The resulting residue was washed with diethyl ether, and the precipitate which formed was then dried in vacuo to give the title compound as a cream solid (2.82 g); ESMS m/z 261.1 [M+H]⁺.

### Intermediate 36: 6-(3-Aminopropyl)-1,4-dihydro-1-ethenyl-4-oxo-quinoline-3-carboxylic acid trifluoroacetate salt

### a) 6-(3-tert-Butoxycarbonylaminopropyl)-1,4-dihydro-1-ethenyl-4-oxo-quinoline-3-carboxylic acid ethyl ester

To a stirred mixture of Intermediate 8b (2.5 g) and ethenyl acetate (16.6 mL) in DMF (10 mL) was added sodium tetrachloropalladate (II) (0.014 g), and the mixture heated to 95° C. After 22 h additional sodium tetrachloropalladate (II) (0.014 g) was added and heating continued for a further 18 h. The mixture was then cooled, filtered, and the filtrate concentrated in vacuo to give a residue which was purified by flash chromatography (silica gel, 0-5% MeOH in DCM) to give the title compound as a white solid (1.84 g); ESMS m/z 401.1 [M+H]⁺.

### b) 6-(3-tert-Butoxycarbonylaminopropyl)-1,4-dihydro-1-methyl-4-oxo-quinoline-3-carboxylic acid

A solution of Intermediate 36a (1.84 g) in THF (30 mL) was treated with 0.2 N aqueous sodium hydroxide (27.5 mL). After 3.5 h the mixture was concentrated in vacuo, and the residue taken up in water and extracted with diethyl ether. The aqueous was then treated with excess solid carbon dioxide, and the resulting precipitate removed by filtration and dried in vacuo to give the title compound as a cream solid (1.34 g); ESMS m/z 373.0 [M+H]⁺.

### c) 6-(3-Aminopropyl)-1,4-dihydro-1-methyl-4-oxo-quinoline-3-carboxylic acid trifluoroacetate salt

A solution of Intermediate 36b (0.414 g) in DCM (6 mL) was treated with TFA (2 mL). After 20 min the solvent was removed in vacuo, the residue taken up in toluene (20 mL), the mixture concentrated in vacuo, then the residue taken up in DCM (20 mL), and concentrated in vacuo to give the title compound as an off-white solid (0.408 g); ESMS m/z 273.1 [M+H]⁺.

### Intermediate 37: 7-Chloro-1-cyclopropyl-6-({2-[(2-hydroxyethyl)oxy]ethyl}amino)-4-oxo-1,4-dihydro-3-quinolinecarboxylic acid methyl ester

### a) 7-Chloro-1-cyclopropyl-6-[2-(2-hydroxy-ethoxy)-ethyl amino]-4-oxo-1,4-dihydro-quinoline-3-carboxylic acid

A mixture of 7-chloro-1-cyclopropyl-6-fluoro-4-oxo-1,4-dihydro-quinoline-3-carboxylic acid (10 g) in 1-methyl-2-pyrrolidinone (70 mL) was treated with 2-(2-amino-ethoxy)-ethanol (18 mL), and the mixture stirred at 110° C. After 24 hours the mixture was diluted with water (200 mL) and DCM (60 mL) and the pH adjusted to 10. The aqueous layer was extracted with DCM (5×50 mL) and then adjusted to pH 6.7. After 10 minutes a precipitate formed, which was filtered off to give the title compound (2.7 g). After standing overnight a second precipitate formed, which was filtered off to give a 1:1 mixture (by LCMS) of the title compound and 1-cyclopropyl-6-fluoro-7-[2-(2-hydroxy-ethoxy)-ethylamino]-4-oxo-1,4-dihydro-quinoline-3-carboxylic acid as yellow solid (7.7 g); ESMS m/z 367.2 [M+H]⁺.

### b) 7-Chloro-1-cyclopropyl-6-({2-[(2-hydroxyethyl)oxy]ethyl}amino)-4-oxo-1,4-dihydro-3-quinolinecarboxylic acid methyl ester

A stirred suspension of Intermediate 37a (1.014 g) in MeOH (20 mL) and DCM (10 mL) was treated with trimethylsilyldiazomethane (2.0 M in hexanes) (1.80 mL). The mixture was stirred for 27 h, during which time further trimethylsilyldiazomethane (2.0M in hexanes) (2.5 mL) was added. The mixture was then concentrated in vacuo to give a residue which was purified by flash chromatography (silica gel, 0-7.5% methanolic ammonia [2M] in DCM) to give the title compound as a yellow solid (0.866 g); ESMS m/z 381.2 [M+H]⁺.

### Intermediate 38: 6-[2-({2-[(2-Aminoethyl)oxy]ethyl}oxy)ethyl]-1-ethyl-4-oxo-1,4-dihydro-3-quinolinecarboxylic acid hydrochloride

### a) 1-Ethyl-6-[2-({2-[(2-hydroxyethyl)oxy]ethyl}oxy)ethyl]-4-oxo-1,4-dihydro-3-quinolinecarboxylic acid ethyl ester

To a stirred mixture of Intermediate 6a (8.55 g), triethylamine (4.82 mL), and 2-{[2-(ethenyloxy)ethyl]oxy}ethanol (6.29 mL) in toluene (25 mL) was added 10% palladium on charcoal (0.245 g) and the mixture heated to 100° C. After 3 h the solvent was removed in vacuo to give a residue which was taken up in EtOAc and filtered through celite. The filtrate was washed with an aqueous solution of sodium dihydrogen phosphate, dried (Na₂SO₄), filtered, and concentrated in vacuo to give a mixture containing 1-ethyl-6-[2-({2-[(2-hydroxyethyl)oxy]ethyl}oxy)ethenyl]-4-oxo-1,4-dihydro-3-quinolinecarboxylic acid ethyl ester as a brown oil (12.13 g), ESMS m/z 376.3 [M+H]⁺ which was used without further purification. A solution of this material in EtOAc (50 mL), and DCM (100 mL) was hydrogenated over 10% palladium on charcoal (2 g) at atmospheric pressure, with additional 10% palladium on charcoal (5 g) added during the course of the reaction. After 72 h the catalyst was removed by filtration, and the filtrate concentrated in vacuo to give a residue which was purified by flash chromatography (silica gel, 0-7% methanolic ammonia [2M] in DCM) to give a mixture (4.21 g) which was taken up DCM (140 mL) and hydrogenated over 10% palladium on charcoal (2 g) at atmospheric pressure for 14 h. Further 10% palladium on charcoal (2.6 g) was then added and the mixture hydrogenated at 45 p.s.i. for 29 h. The mixture was then filtered and concentrated in vacuo to give a residue which was purified by flash chromatography (silica gel, 0-6% methanolic ammonia [2M] in DCM) to give a mixture which was taken up in EtOAc, washed with an aqueous solution of sodium dihydrogen phosphate, dried (Na₂SO₄), filtered, and concentrated in vacuo to give the title compound as a yellow/brown oil (2.15 g); ESMS m/z 378.2 [M+H]⁺.

### b) 6-(10,10-Dioxido-3,6,9-trioxa-10-thiaundec-1-yl)-1-ethyl-4-oxo-1,4-dihydro-3-quinolinecarboxylic acid ethyl ester

A solution of Intermediate 38b (1.58 g) in DCM (30 mL) at 0° C. was treated with triethylamine (0.99 mL), followed by methanesulfonyl chloride (0.42 mL), and the mixture stirred for 2 h. Saturated sodium hydrogen carbonate solution (20 mL) was then added and the organic solvent removed in vacuo. The aqueous mixture was adjusted to pH 11 by the addition of an aqueous solution of sodium carbonate, then extracted with EtOAc. The organic layers were combined, dried (Na₂SO₄), filtered, and concentrated in vacuo to give the title compound as a pale yellow gum (2.04 g); ESMS m/z 456.3 [M+H]⁺.

### c) 6-[2-({2-[(2-Azidoethyl)oxy]ethyl}oxy)ethyl]-1-ethyl-4-oxo-1,4-dihydro-3-quinolinecarboxylic acid ethyl ester

A solution of Intermediate 38c (1.9 g) in DCM (20 mL) was treated with 1,1,3,3-tetramethylguanidinium azide (1.95 g) and stirred at room temperature for 21 h, then at reflux for 27 h. Additional 1,1,3,3-tetramethylguanidinium azide (0.3 g) was added, and the mixture heated at reflux for a further 8 h. The mixture was concentrated in vacuo to give a residue which was taken up in EtOAc, washed with water, dried (Na₂SO₄), filtered, and concentrated in vacuo to give a residue which was purified by flash chromatography (silica gel, 0-6% methanolic ammonia [2M] in DCM) to give the title compound as a colourless gum (1.49 g); ESMS m/z 403.3 [M+H]⁺.

### d) 6-[2-({2-[(2-Azidoethyl)oxy]ethyl}oxy)ethyl]-1-ethyl-4-oxo-1,4-dihydro-3-quinolinecarboxylic acid

A solution of Intermediate 38d (1.47 g) in 1,4-dioxane (20 mL) was treated with 2 N aqueous sodium hydroxide (3.64 mL). After 20 h the mixture was concentrated in vacuo to give a residue which was taken up in water, and treated with excess solid carbon dioxide. The resulting precipitate was removed by filtration and dried in vacuo to give the title compound as a cream solid (1.09 g); ESMS m/z 375.2 [M+H]⁺.

### e) 6-[2-({2-[(2-Aminoethyl)oxy]ethyl}poxy)ethyl]-1-ethyl-4-oxo-1,4-dihydro-3-quinolinecarboxylic acid hydrochloride

A solution of Intermediate 38e (1.07 g) in THF (30 mL) was treated with triphenylphosphine (1.5 g) and stirred for 20 min. Water (2 mL) was added and stirring continued for 21 h. The solvent was then removed in vacuo to give a residue which was taken up in hydrochloric acid (2 N) and washed with EtOAc. The aqueous solution was concentrated in vacuo to give a residue which was taken up in water and the solution freeze dried to give the title compound as a cream solid (0.63 g); ESMS m/z 349.3 [M+H]⁺.

### Intermediate 39: 9-(3-Aminopropyl)-7-oxo-1H,7H-[1,3]oxazino[5,4,3-ij]quinoline-6-carboxylic acid trifluoroacetate salt

### a) Ethyl 9-Iodo-7-oxo-1H,7H-[1,3]oxazino[5,4,3-ij]quinoline-6-carboxylate

A suspension of Intermediate 11d (0.44 g) and potassium carbonate 0.488 g) in DMF (8 mL) was treated with chloroiodomethane (0.8 mL) and the resultant mixture heated at 100° C. After 17 h the reaction was cooled and evaporated to yield the crude product. Chromatography on silica gel eluting with 0-5% MeOH in DCM gave the title compound as a pale green solid (0.30 g); ESMS m/z 386.0 [M+H]⁺.

### b) Ethyl 9-(3-tert-Butoxycarbonylamino-prop-1-ynyl-7-oxo-1H,7H-[1,3]oxazino[5,4,3-ij]quinoline-6-carboxylate

A mixture of Intermediate 39a (0.297 g), copper (I) iodide (0.016 g) and triethylamine (3.75 mL) were suspended in dry MeCN (8 mL). The suspension was heated to 50° C. whilst argon was bubbled through. After 20 min, dichlorobis(triphenylphosphine)palladium (II) (0.016 g) and N-tert-butoxycarbonylpropargylamine (0.203 g) were added and the mixture was heated at 50° C. for 1.5 h. The reaction mixture was cooled, concentrated in vacuo and purified by chromatography (silica gel, 0 to 5% MeOH in DCM) to give the title compound as a beige solid (0.23 g); ESMS m/z 413.2 [M+H]⁺.

### c) Ethyl 9-(3-tert-Butoxycarbonylaminopropyl)-7-oxo-1H,7H-[1,3]oxazino[5,4,3-ij]quinoline-6-carboxylate

A solution of Intermediate 39b (0.225 g) in DCM (10 mL) and MeOH (10 mL) was treated with 10% palladium on carbon (0.1 g) and hydrogenated at room temperature and atmospheric pressure for 16 h. The reaction mixture was filtered and concentrated to give the title compound as a yellow solid (0.21 g); ESMS m/z 417.2 [M+H]⁺.

### d) 9-(3-tert-Butoxycarbonylaminopropyl)-7-oxo-1H,7H-[1,3]oxazino[5,4,3-ij]quinoline-6-carboxylic acid sodium salt

A solution of Intermediate 39c (0.205 g) in THF (5 mL) was treated with 2M sodium hydroxide (0.40 mL). The reaction was stirred for 17 h at 50° C. then solid carbon dioxide was added and the reaction mixture was concentrated to give the title compound as a pale yellow solid (0.21 g); ESMS m/z 389.1 [M+H]⁺.

### e) 9-(3-Aminopropyl)-7-oxo-1H,7H-[1,3]oxazino[5,4,3-ij]quinoline-6-carboxylic acid trifluoroacetate salt

A solution of Intermediate 39d (0.2 g) in DCM (3 mL) was treated with TFA (3 mL) and the reaction stirred at room temperature for 20 min. The solution was evaporated to dryness to give the title compound as a white solid (0.2 g); ESMS m/z 289.1 [M+H]⁺.

### Intermediate 40: 6-(3-Aminopropyl)-1-methoxy-4-oxo-1,4-dihydro-3-quinolinecarboxylic acid hydrochloride

### a) 1-(Methoxy)-6-iodo-4-oxo-1,4-dihydroquinoline-3-carboxylic acid ethyl ester

A mixture of Intermediate 4a (2.5 g), O-methylhydroxylamine hydrochloride (0.64 g) and potassium carbonate (2.21 g) in DMF (50 mL) was stirred at room temperature for 1 h then at 100° C. for 1 h and then cooled. The mixture was poured into water, the solid filtered off, then washed with water and dried. The solid was purified by flash chromatography (silica gel, 0-5% MeOH in DCM) to give the title compound as a white solid (1.75 g); APCI m/z 374.0 [M+H]⁺.

### b) 6-[3-(tert-Butoxycarbonylamino)-1-propyn-1-yl]-1-methoxy-4-oxo-1,4-dihydroquinoline-3-carboxylic acid ethyl ester

A mixture of Intermediate 40a (1.94 g), copper (I) iodide (0.099 g) and triethylamine (24 mL) were suspended in dry MeCN (48 mL). The light green suspension was degassed and stirred under argon at room temperature for 15 minutes. Dichlorobis(triphenylphosphine)palladium (II) (0.118 g) and N-tert-butoxycarbonylpropargylamine (1.21 g) were added, the mixture was stirred for 1 h and evaporated. The residue was taken up in DCM and washed with water. The organic phase was dried and evaporated to give a dark solid which was purified by flash chromatography (silica gel, 20-50% {EtOAc in DCM then 5% MeOH in DCM) to give the title compound as a pale yellow solid (1.04 g); APCI m/z 401.1 [M+H]⁺.

### c) 6-(3-tert-Butoxycarbonylaminopropyl)-1-methoxy-4-oxo-1,4-dihydro-quinoline-3-carboxylic acid ethyl ester

Intermediate 40b (1.04 g) in DCM (30 mL) was hydrogenated over 10% palladium on charcoal (0.05 g) for 1.5 h. The resultant mixture was filtered and the solvent evaporated to give the title compound as a yellow solid (1 g); APCI m/z 405.1 [M+H]⁺.

### d) 6-(3-tert-Butoxycarbonylaminopropyl)-1-methoxy-4-oxo-1,4-dihydro-quinoline-3-carboxylic acid

Intermediate 40c (1 g) was dissolved in MeOH (10 mL) and treated with 1N aqueous sodium hydroxide (3.7 mL) and stirred overnight at room temperature. The solution was evaporated to low volume and then acidified with 5% citric acid. The mixture was extracted with DCM. The DCM solution was washed with water, dried (Na₂SO₄) and evaporated to give the title compound as a yellow solid (0.82 g); APCI m/z 399.0 [M+Na]⁺.

### e) 6-(3-Aminopropyl)-1-methoxy-4-oxo-1,4-dihydro-quinoline-3-carboxylic acid hydrochloride

A solution of Intermediate 40d (0.82 g) in DCM (10 mL) at was treated with 4M HCl in 1,4-dioxan (5 mL). After 2 h the mixture was evaporated. The residue was triturated with acetone. The solid was filtered off, washed with acetone and dried to give the title compound as a grey solid (0.57 g); APCI m/z 277.0 [M+H]⁺.

### Intermediate 41: 7-(2-Amino-ethoxy)-1-ethyl-4-oxo-1,4-dihydro-quinoline-3-carboxylic acid sodium salt

### a) 7-Benzyloxy-1-ethyl-4-oxo-1,4-dihydro-quinoline-3-carboxylic acid ethyl ester

A mixture of 7-benzyloxy-4-oxo-1,4-dihydro-quinoline-3-carboxylic acid ethyl ester (0.97 g) and potassium carbonate (0.56 g) in DMF was stirred for 1 h at 50° C. under argon followed by addition of Iodoethane (0.9 g). After stirring at 50° C. for a further 14 h the mixture was cooled and the DMF evaporated. The residue was treated with water and cooled in ice. The resultant crystalline product was filtered and dried under vacuum overnight to yield the title compound as a white powder; ¹H NMR δ (CDCl₃) 1.42 (3H, t, J=7.2 Hz), 1.45 (3H, t, J=7.2 Hz), 4.14 (2H, q, J=7.2 Hz), 4.39 (2H, q, J=7.1 Hz), 5.20 (2H, s), 6.86 (1H, d, J=2.2 Hz), 7.11 (1H, dd, J=9.0 & 2.2 Hz), 7.3-7.5 (5H, m), 8.42 (1H, s), 8.47 (1H, d, J=9.0 Hz).

### b) 1-Ethyl-7-hydroxy-4-oxo-1,4-dihydro-quinoline-3-carboxylic acid ethyl ester

A solution of Intermediate 41a (1 g) in MeOH (10 mL) was hydrogenated in the presence of 10% palladium on charcoal (0.05 g) at 1 atmosphere and room temperature. After 14 h another 0.05 g of catalyst was added. After a further 24 h the mixture was filtered and the MeOH evaporated to yield the title compound as a pale yellow solid; ¹H NMR δ [(CD₃)₂SO] 1.28 (3H, t, J=7.1 Hz), 1.36 (3H, t, J=7.1 Hz), 4.20 (2H, q, J=7.1 Hz), 4.28 (2H, q, J=7.1 Hz), 6.92 (1H, dd, J=8.8 & 2.1 Hz), 6.97 (1H, d, J=2.1 Hz), 8.08 (1H, d, J=8.8 Hz), 8.57 (1H, s); 10.52 (1H, br. s).

### c) 7-(2-Dibenzylamino-ethoxy)-1-ethyl-4-oxo-1,4-dihydro-quinoline-3-carboxylic acid ethyl ester

Intermediate 41b (0.371 g) was dissolved in dry DMF (10 mL) and to this was added potassium carbonate (0.588 g) and N,N-dibenzyl-(2-chloroethyl)amine hydrochloride (0.462 g). The mixture was heated at 70° C. for 5 h, evaporated to a small volume, diluted with water and extracted with EtOAc (×2). The combined organic extracts were washed with brine, dried and evaporated under reduced pressure to give a yellow oil (0.76 g). This residue was purified by chromatography on silica gel (40 g), eluting with 0-4% MeOH in DCM, to give the title compound as a cream solid (0.709 g); ESMS m/z 485.3 [M+H]⁺ (100%).

### d) 7-(2-Amino-ethoxy)-1-ethyl-4-oxo-1,4-dihydro-quinoline-3-carboxylic acid ethyl ester

A solution of Intermediate 41c (0.7 g) in ethanol (70 mL) was hydrogenated over 20% palladium(II) hydroxide on carbon (0.26 g) at 50 psi for 31 h. More catalyst (0.2 g) was then added and hydrogenation continued for a further 22 h. The mixture was then filtered through kieselguhr, washing well with ethanol, and the filtrate evaporated to dryness under reduced pressure. The residue was purified by chromatography on silica gel (20 g), eluting with 0-8% methanolic ammonia (2M) in DCM, to give the title compound as an off-white solid (0.239 g); ESMS m/z 305.3 [M+H]⁺.

### e) 7-(2-Amino-ethoxy)-1-ethyl-4-oxo-1,4-dihydro-quinoline-3-carboxylic acid sodium salt

Intermediate 41d (0.225 g) was dissolved in THF (10 mL) and 1,4-dioxane (10 mL), then aqueous sodium hydroxide (0.2N, 3.7 mL) was added, and the mixture stirred for 18 h. Solid carbon dioxide was then added and the solution evaporated to dryness under reduced pressure to give the title compound as a pale yellow solid (0.212 g); ESMS m/z 277.2 [M+H]⁺ for free acid.

### Intermediate 42: 9-[(2-Aminoethyl)amino]-5-methyl-1-oxo-6,7-dihydro-1H,5H-pyrido[3,2,1-ij]quinoline-2-carboxylic acid formate salt

A suspension of flumequine (0.522 g) and 1,2-diaminoethane (0.668 mL) in N-methylpyrrolidinone (1 mL) was microwaved at 180° C. for 1.5 h. The reaction mixture was purified by mass directed automatic preparative HPLC to give the title compound as a yellow solid (0.35 g); ESMS m/z 302.3 [M+H]⁺.

### Intermediate 43: 742-Amino-ethylamino)-1-ethyl-4-oxo-1,4-dihydro-[1,8]naphthyridine-3-carboxylic acid trifluoroacetate

### a) 7-(2-tert-Butoxycarbonylamino-ethylamino)-1-ethyl-6-fluoro-4-oxo-1,4-dihydro-[1,8]naphthyridine-3-carboxylic acid

7-Chloro-1-ethyl-4-oxo-1,4-dihydro-[1,8]naphthyridine-3-carboxylic acid (U.S. Pat. No. 3,149,104) (1 g) in MeCN (30 mL) was treated with triethylamine (1.12 mL), followed by (2-amino-ethyl)-carbamic acid tert-butyl ester (1.26 mL) and the mixture heated to reflux. After 2 h the reaction was cooled to 20° C. and the solvent removed in vacuo. The residue was triturated with water, EtOAc and DCM. The EtOAc and DCM soluble material was purified by chromatography (silica gel, 0-100% EtOAc in DCM) to give the title compound as a cream solid, 0.12 g. ESMS m/z 377.0 [M+H]⁺. The water, EtOAc and DCM insoluble material was slurried in MeOH, filtered and dried to give a further 0.72 g of less pure title compound as a tan solid ESMS m/z 377.3 [M+H]⁺.

### b) 7-(2-Amino-ethylamino)-1-ethyl-4-oxo-1,4-dihydro-[1,8]naphthyridine-3-carboxylic acid trifluoroacetate

The less pure Intermediate 43a (0.72 g) was suspended in DCM (20 mL), TFA (5 mL) added, and the solution stirred for 20 min. The mixture was concentrated in vacuo, and again from toluene (30 mL), and triturated with THF to give the title compound as a tan solid (0.7 g); ESMS m/z 277.0 [M+H]⁺.

### Intermediate 44: 4″-Vinylsulfonyl-erythromycin A (9E)-O-methyloxime

### a) 2′-O-Acetyl-erythromycin A (9E)-O-methyloxime

A solution of erythromycin (9E)-O-methyloxime (5.7 g) in DCM (70 mL) was treated with triethylamine (2.25 mL) followed by acetic anhydride (1.18 mL). After stirring overnight at room temperature the mixture was diluted with DCM and washed with aqueous sodium bicarbonate. The organic layer was separated, dried and evaporated to yield the title product as a solid; ESMS m/z 805.8 [M+H⁺].

### b) 2′-O-Acetyl-4″-vinylsulfonyl-erythromycin A (9E)-O-methyloxime

To a solution of Intermediate 44a (6.07 g) in toluene (90 mL) was added triethylamine (3.14 mL) and 2-chloro-1-ethanesulfonyl chloride (1.18 mL) at 20° C. under argon. After 30 min the mixture was filtered and the filtrate diluted with EtOAc (100 mL), washed with saturated aqueous sodium hydrogen carbonate, water, and brine, dried (Na₂SO₄) and evaporated. The residue was purified by flash chromatography (silica gel, 0-4% % [9:1 MeOH/20M aqueous ammonia] in DCM) to give the title compound (5.92 g); ESMS m/z 895.6 [M+H]⁺.

### c) 4″-Vinylsulfonyl-erythromycin A (9E)-O-methyloxime

A solution of Intermediate 44b (5.91 g) in MeOH (60 mL) was stirred at 55° C. for 16 h. The mixture was cooled, concentrated in vacuo and the residue purified by flash chromatography (silica gel, 10-11% MeOH in DCM) to give the title compound, (4.09 g); ESMS m/z 853.6 [M+H]⁺.

### Intermediate 45: 6-[3-(2-Amino-ethoxy)-propyl]-1-ethyl-4-oxo-1,4-dihydro-quinoline-3-carboxylic acid trifluoroacetate salt

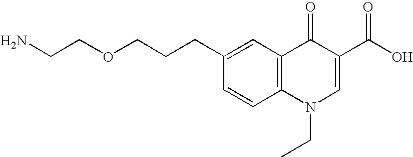

### a) 6-[3-(2-tert-Butoxycarbonylamino-ethoxy)-prop-1-ynyl]-1-ethyl-4-oxo-1,4-dihydro-quinoline-3-carboxylic acid ethyl ester

A mixture of Intermediate 6a (1.18 g) and copper (I) iodide (0.067 g) suspended in MeCN (100 mL) and triethylamine (156 mL) was deoxygenated with argon for 30 min. The mixture was then treated with tert-butyl [2-(2-propyn-1-yloxy)ethyl]carbamate (1.08 g) and bis(triphenylphosphine)palladium (II) dichloride (0.67 g) and stirred for 16 h. The was mixture evaporated to dryness and the residue purified by flash chromatography (silica gel, 0-10% MeOH in DCM) to give the title compound as a beige solid (0.592 g); ESMS m/z 443.4 [M+H]⁺.

### b) 6-[3-(2-tert-Butoxycarbonylamino-ethoxy)-propyl]-1-ethyl-4-oxo-1,4-dihydro-quinoline-3-carboxylic acid ethyl ester

A mixture of Intermediate 45a (0.592 g) and 10% palladium on charcoal (0.18 g) in MeOH (120 mL) was hydrogenated at room temperature. After 16 h the mixture was filtered and the solvent evaporated to give the title compound as a beige solid (0.48 g); ESMS m/z 447.3 [M+H]⁺.

### c) 6-[3-(2-tert-Butoxycarbonylamino-ethoxy)-propyl]-1-ethyl-4-oxo-1,4-dihydro-quinoline-3-carboxylic acid sodium salt

A solution of Intermediate 45b (0.481 g) in THF (13 mL) was treated with 2M sodium hydroxide (0.59 mL). After stirring at 50° C. for 16 h the mixture was treated with solid carbon dioxide then concentrated to yield the title compound as a beige solid (0.475 g); ESMS m/z 419.4 [M+H]⁺.

### d) 6-[3-(2-Amino-ethoxy)-propyl]-1-ethyl-4-oxo-1,4-dihydro-quinoline-3-carboxylic acid trifluoroacetate salt

Intermediate 45c (0.451 g) in DCM (5 mL) was treated with TFA (5 mL). After stirring at room temperature for 1 h the mixture was evaporated to yield the title compound as a brown oil (0.46 g); ESMS m/z 319.3 [M+H]⁺.

### Intermediate 46: 2-propen-1-yl 1-ethyl-6-{3-[(2-hydroxyethyl)oxy]propyl}-4-oxo-1,4-dihydro-3-quinolinecarboxylate

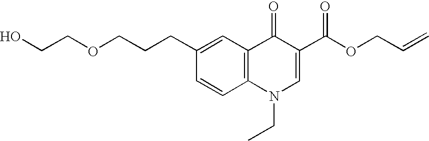

### a) ({[2-(2-propyn-1-yloxy)ethyl]oxy}methyl)benzene

A solution of 2-benzyloxyethanol (25 mL) in THF (120 mL) was cooled to 0° C. under a nitrogen atmosphere and to this solution sodium hydride (60% in oil, 7.4 g) was added. The reaction mixture was heated at 35° C. for 45 min, cooled to 0° C., and then 3-bromo-propyne (27.44 mL) was added. Reaction mixture was stirred at room temperature overnight and then partitioned between EtOAc and water. The combined organic layers were dried (Na₂SO₄), filtered, and concentrated in vacuo to give the title compound (24.8 g); δ_{H} (300 MHz; DMSO-d₆) 2.43 (1H, t), 3.63-3.67 (2H, m), 3.70-3.74 (2H, m), 4.21 (2H, d), 4.57 (2H, s), 7.33-7.35 (5H, m).

### b) 1-ethyl-6-Iodo-4-oxo-1,4-dihydro-3-quinolinecarboxylic acid

Intermediate 6a (11.8 g) and sodium hydroxide (5.9 g) were dissolved in THF/water (1:1) (300 mL). Reaction mixture was stirred and heated for 3 h at 80° C. THF was evaporated at reduced pressure, and to the residual solution water (100 mL) and DCM (60 mL) were added, and mixture adjusted to pH 12 with sodium hydroxide. Layers were separated and the pH-value of the aqueous was adjusted to 4.5 with diluted hydrochloric acid. A precipitate formed, which was removed by filtration, washed with water, and dried in vacuo at 50° C. for 4 h to give the title compound as white crystals (10.6 g); δ_{H} (300 MHz; DMSO-d₆) 1.42 (3H, t), 4.58 (2H, q), 7.84 (1H, d), 8.20 (1H, dd), 8.57 (1H, d), 9.06 (1H, s); ESMS m/z 344.1 [M+H]⁺.

### c) 1-Ethyl-4-oxo-6-[3-({2-[(phenylmethyl)oxy]ethyl}oxy)-1-propyn-1-yl]-1,4-dihydro-3-quinolinecarboxylic acid

To a solution of Intermediate 46b (10.6 g) in MeCN/water (1:1) (250 mL), was added copper (I) iodide (0.571 g) and the mixture stirred for 20 min at 50° C. Then a solution of Pd(PPh₃)₂Cl₂ (1.05 g) and Intermediate 46a (10.26 g) in MeCN/water (1:1) (40 mL) was added to the reaction mixture and stirred at 50° C. overnight. Organic solvents were evaporated and water (500 mL) was added to the residue, pH was adjusted to 12 and the water layer extracted with diisopropyl-ether (3×150 mL). Charcoal was added to the water layer, the mixture stirred for 15 min, and then filtered over Celite. After the pH was adjusted to 6a precipitate formed, which was filtered off and dried to give the title compound as a solid (10.1 g); δ_{H} (300 MHz; CDCl₃) 1.59 (3H, t), 3.69-3.72 (2H, m), 3.80-3.82 (2H, m), 4.39 (2H, q), 4.48 (2H, s), 4.60 (2H, s), 7.27-7.38 (5H, m), 7.57 (1H, d), 7.83 (1H, dd), 8.57 (1H, d), 8.78 (1H, s); ESMS m/z 406.3 [M+H]⁺.

### d) 1-Ethyl-6-{3-[(2-hydroxyethyl)oxy]propyl}-4-oxo-1,4-dihydro-3-quinolinecarboxylic acid

A mixture of Intermediate 46c (10 g) and 10% palladium on charcoal (3 g) in MeOH (150 mL) and DCM (50 mL) was hydrogenated at 5 bar for 48 h. The catalyst was removed by filtration, and the filtrate concentrated in vacuo to give the title compound (6.4 g); δ_{H} (300 MHz; DMSO-d₆) 1.43 (3H, t), 1.85-1.95 (2H, m), 2.86 (2H, t), 3.39-3.43 (4H, m), 3.51 (2H, t), 4.62 (2H, q), 7.84 (1H, d), 7.98 (1H, d), 8.19 (1H, s), 9.02 (1H, s); ESMS m/z 320.3 [M+H]⁺.

### e) 2-Propen-1-yl 1-ethyl-6-{3-[(2-hydroxyethyl)oxy]propyl}-4-oxo-1,4-dihydro-3-quinolinecarboxylate

A suspension of Intermediate 46d (1.019 g), and potassium carbonate (1.1 g) in DMF (15 mL) was treated with allyl bromide (0.304 mL) and heated to 50° C. After 23.5 h additional allyl bromide (0.060 mL) was added. Potassium carbonate (0.3 g), followed by allyl bromide (0.060 mL) was added after an additional 3.5 h, and heating continued for 40 min. The solvent was then removed in vacuo to give a residue which was taken up in water, the pH was adjusted to 12 by addition of potassium carbonate, and the mixture extracted with DCM. The combined organic extracts were dried (Na₂SO₄), filtered, and concentrated in vacuo to give a residue which was purified by flash chromatography (silica gel, 0.5-7% methanolic ammonia [2M] in DCM) to give the title compound as a white solid (0.575 g); ESMS m/z 360.3 [M+H]⁺.

### Intermediate 47: 2-propen-1-yl 1-cyclopropyl-6-(3-[(2-hydroxyethyl)oxy]propyl)-4-oxo-1,4-dihydro-3-quinolinecarboxylate

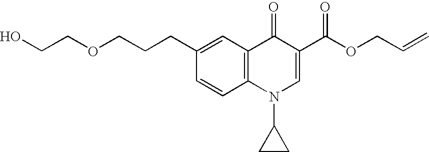

### a) 1-Cyclopropyl-6-iodo-4-oxo-1,4-dihydro-3-quinolinecarboxylic acid

Ethyl 1-cyclopropyl-6-iodo-4-oxo-1,4-dihydro-3-uinolinecarboxylate (S. Turner, J. Strohbach, S. Thaisrivongs, V. Vaillancourt, M. Schnute, and J. Tucker in WO 00/40561), (2.6 g) was dissolved in THF (50 mL) and treated with a solution of sodium hydroxide (1.25 g) in water (50 mL). The reaction mixture was stirred on a magnetic stirrer with heating at 80° C. for 3 h. The THF was evaporated in vacuo, water (50 mL) and DCM (30 mL) were added and the pH adjusted to 12. After the layers were separated, the water layer pH was adjusted to 5. The precipitate which formed was removed by filtration, washed with water, and dried in vacuo at 50° C. for 4 h to give the title compound as a white solid (2.3 g); δ_{H} (300 MHz; DMSO-d₆) 1.18 (2H, m), 1.29 (2H, m), 3.84 (1H, m), 8.06 (1H, d), 8.27 (1H, dd), 8.57 (1H, d), 8.75 (1H, s).

### b) 1-Cyclopropyl-4-oxo-6-[3-({2-[(phenylmethyl)oxy]ethyl}oxy)-1-propyn-1-yl]-1,4-dihydro-3-quinolinecarboxylic acid

To a solution of Intermediate 47a (2.3 g) in MeCN (27 mL) was added copper (I) iodide (0.123 g) and triethylamine (27 mL), and the mixture stirrred for 20 min and then heated at 50° C. Intermediate 46a (2.47 g) and Pd(PPh₃)₂Cl₂ (0.228 g) were added to the reaction mixture, and heating continued at 50° C. overnight. After 24 h, when starting compound was fully converted, MeCN and triethylamine were evaporated in vacuo, water (100 mL) and diisopropylether (40 mL) were added and the pH of the suspension was adjusted to 12 with 40% sodium hydroxide. The layers were separated, the aqueous heated with actived charcoal (0.3 g) at 80° C. for 10 min, and then filtered through Celite. When cooled, the pH was adjusted to 4 with 6N hydrochloric acid. The precipitate which formed was filtered off and dried in vacuo at 50° C. for 4 h to give the title compound as a solid (2.1 g); ESMS m/z 418.0 [M+H]⁺.

### c) 1-Cyclopropyl-6-{3-[(2-hydroxyethyl)oxy]propyl}-4-oxo-1,4-dihydro-3-quinolinecarboxylic acid

A mixture of Intermediate 47b (2.1 g) and 10% palladium on charcoal (1 g) in MeOH (80 mL) and DCM (20 mL) was hydrogenated at 5 bar for 48 h. The catalyst was removed by filtration, and the filtrate concentrated in vacuo to give the title compound as a white solid (1.5 g); δ_{H} (300 MHz; DMSO-d₆) 1.19 (2H, m), 1.32 (2H, m), 1.87 (2H, m), 2.85 (2H, t), 3.39-3.43 (4H, m), 3.52 (2H, t), 3.83 (1H, m), 7.88 (1H, dd), 8.16 (1H, d), 8.22 (1H, d), 8.72 (1H, s); ESMS m/z 332.0 [M+H]⁺.

### d) 2-propen-1-yl 1-cyclopropyl-6-{3-[(2-hydroxyethyl)oxy]propyl}-4-oxo-1,4-dihydro-3-quinolinecarboxylate

A suspension of Intermediate 47c (1.007 g), and potassium carbonate (1.05 g) in DMF (15 mL) was treated with allyl bromide (0.289 mL) and heated to 50° C. After 19.5 h the solvent was removed in vacuo to give a residue which was taken up in water, the pH was adjusted to 12 by addition of potassium carbonate, and the mixture extracted with DCM. The combined organic extracts were dried (Na₂SO₄), filtered, and concentrated in vacuo to give a residue which was purified by flash chromatography (silica gel, 0.5-7% methanolic ammonia [2M] in DCM) to give the title compound as a white solid (0.668 g);

ESMS m/z 372.3 [M+H]⁺.

### Intermediate 48: Phenylmethyl 7-chloro-1-cyclopropyl-6-({2-[(2-hydroxyethyl)oxy]ethyl}amino)-4-oxo-1,4-dihydro-3-guinolinecarboxylate

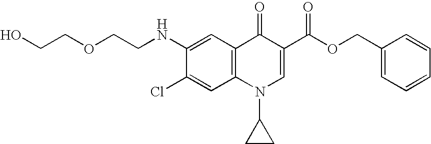

To a stirred suspension of Intermediate 37a (0.528 g) in DCM (10 mL) was added phenyldiazomethane in toluene (0.255 M) (5.64 mL) (Guifa Su et al. Syn. Comm; 2003, 33, 16, 2873-2884). After 10 min the mixture was heated to 40° C., then additional phenyldiazomethane in toluene (0.255 M) (5.64 mL) added after 2.5 h. After 10 min a portion of the mixture (5 mL) was removed and to it was added MeOH (1 mL), followed by phenyldiazomethane in toluene (0.255 M) (5 mL) after 30 min. Both mixtures were allowed to cool to room temperature. After an additional 14.5 h the two portions were combined and MeOH (2 mL) and DCM (10 mL) was added to obtain a solution, then phenyldiazomethane in toluene (0.255 M) (10 mL) added. The mixture was kept between room temperature and 40° C. for 100 h, during which time additional phenyldiazomethane in toluene (0.255 M) (6 mL) was added. The mixture was then concentrated in vacuo and purified by flash chromatography (silica gel, 0-6% methanolic ammonia [2M] in DCM) to give the title compound as a pale yellow solid (0.51 g); ESMS m/z 457.2 [M+H]⁺.

### Intermediate 49: 9-Deoxy-9a-allyloxycarbonyl-4″-O-vinylsulfonyl-9a-aza-9a-homoerythromycin A

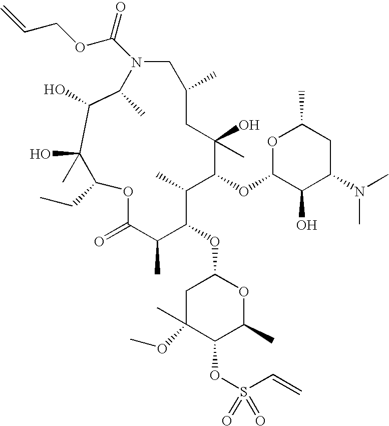

### a) 2′-O-Acetyl-9-deoxy-9a-allyloxycarbonyl-9a-aza-9a-homoerythromycin A

To an ice-cooled mixture of 2′-O-acetyl-9-deoxy-9a-aza-9a-homoerythromycin A (G. Bright in EP 0109253) (6.59 g) and sodium hydrogen carbonate (2.85 g) in THF (85 mL) and water (21 mL) was added allyl chloroformate (1.08 mL). The mixture was stirred for 15 min, then allowed to warm to room temperature. Additional allyl chloroformate (0.54 mL) was added after 2 h. Further allyl chloroformate (0.50 mL) was added after a further 1 h. After 10 min the mixture was concentrated in vacuo, the resulting residue taken up in DCM, and washed with an aqueous solution of sodium hydrogen carbonate. The organic extracts were dried (Na₂SO₄), filtered, and concentrated in vacuo to give a residue which was purified by flash chromatography (silica gel, 0.5-7% methanolic ammonia [2M] in DCM) to give the title compound as a white solid (5.41 g); ESMS m/z 861.7 [M+H]⁺.

### b) 2′-O-Acetyl-9-deoxy-9a-allyloxycarbonyl-4″-O-vinylsulfonyl-9a-aza-9a-homoerythromycin A

To a mixture of Intermediate 49a (5.17 g) and triethylamine (2.51 mL) in toluene (105 mL) was added 2-chloro-1-ethanesulfonyl chloride (0.94 mL) dropwise. After 45 min triethylamine (0.42 mL) was added, followed by 2-chloro-1-ethanesulfonyl chloride (0.16 mL). After 20 min the mixture was treated with a saturated sodium bicarbonate solution (100 mL), then extracted with EtOAc. The organic extracts were combined, dried (Na₂SO₄), filtered, and concentrated in vacuo to give a residue which was purified by flash chromatography (silica gel, 0-7% MeOH in DCM) to give the title compound as a white solid (3.55 g); ESMS m/z 951.7 [M+H]⁺.

### c) 9-Deoxy-9a-allyloxycarbonyl-4″-O-vinylsulfonyl-9a-aza-9a-homoerythromycin A

A stirred solution of Intermediate 49b (3.36 g) in MeOH (200 mL) was heated to 60° C. After 4 h the mixture was cooled to 50° C. and heating continued for a further 13 h. The solvent was then removed in vacuo to give a solid which was taken up in toluene and DCM and concentrated in vacuo to give the crude title compound as a white solid (3.43 g) which was used without further purification; ESMS m/z 909.6 [M+H]⁺.

### Intermediate 50: 2′-O-Allyloxycarbonyl-9-deoxy-9a-allyloxycarbonyl-4″-O vinylsulfonyl-9a-aza-9a-homoerythromycin A

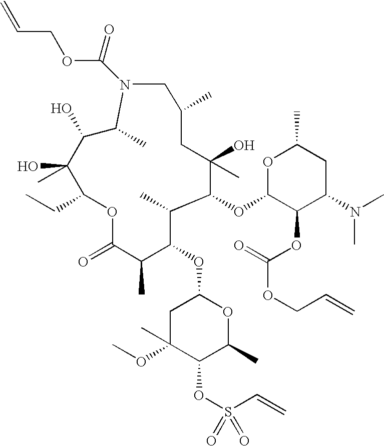

### a) 2′-O-Allyloxycarbonyl-9-deoxy-9a-allyloxycarbonyl-9a-aza-9a-homoerythromycin A

To an ice-cooled mixture of 9-deoxy-9a-aza-9a-homoerythromycin A (S. Djokic et al. in DE 3012533), (109.3 g) and potassium carbonate (82.2 g) in THF (500 mL) and water (250 mL), with overhead stirring, was added allyl chloroformate (34.7 mL) dropwise, keeping the internal temperature below 3° C. Additional allyl chloroformate (15.8 mL) was added after 20 min. Further allyl chloroformate (7.89 mL) was added after a further 30 min. After 50 min the organic layer was separated, washed with brine (100 mL), dried (Na₂SO₄), filtered, and concentrated in vacuo. The aqueous layer was re-extracted with Et₂O (500 mL) (×3) and the organic extracts combined, dried (Na₂SO₄), filtered, and concentrated in vacuo. These Et₂O extracts were combined with the first organic extracts to give the title compound as a white solid (140 g); ESMS m/z 903.8 [M+H]⁺.

### b) 2′-O-Allyloxycarbonyl-9-deoxy-9a-allyloxycarbonyl-4″-O-vinylsulfonyl-ga-aza-9a-homoerythromycin A

To a stirred mixture of Intermediate 50a (99.2 g) and triethylamine (45.9 mL) in toluene (800 mL) was added 2-chloro-1-ethanesulfonyl chloride (13.7 mL) dropwise, keeping the internal temperature between 18° C. and 24° C. by cooling the flask in ice-water. After 45 min triethylamine (15.3 mL) was added, followed by 2-chloro-1-ethanesulfonyl chloride (5.7 mL). After 1 h the mixture was treated with a saturated sodium bicarbonate solution (300 mL), and the layers separated. The organic layer was dried (Na₂SO₄), filtered, and concentrated in vacuo. The aqueous layer was re-extracted with EtOAc (750 mL) (×2) and the organic extracts combined, dried (Na₂SO₄), filtered, and concentrated in vacuo. These EtOAc extracts were combined with the first organic extracts to give a solid (114 g). A portion of this material (56.1 g) was purified by flash chromatography (silica gel, 0-3% MeOH in DCM) to give the crude title compound as a white solid (39.4 g) which was used without further purification; ESMS m/z 993.8 [M+H]⁺.

### Intermediate 51: 9-Deoxy-9a-benzyloxycarbonyl-4″-O-vinylsulfonyl-9a-aza-9a-homoerythromycin A

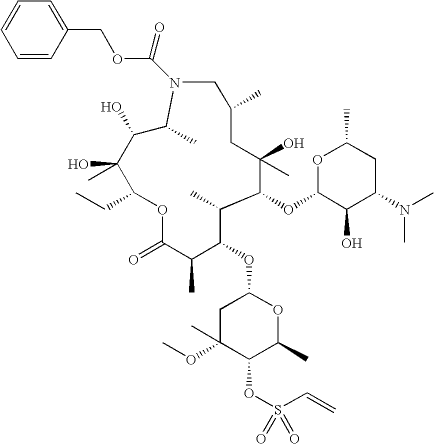

### a) 2′-O-Benzyloxycarbonyl-9-deoxy-9a-benzyloxycarbonyl-4″-O-vinylsulfonyl-9a-aza-9a-homoerythromycin A

A stirred mixture of 2′-O-Benzyloxycarbonyl-9-deoxy-9a-benzyloxycarbonyl-9a-aza-9a-homoerythromycin A (S. Djokic et al. in Journal of Antibiotics, 1993, 46(8), 1239-1245), (8.6 g), and triethylamine (3.59 mL) in toluene (100 mL) was treated with 2-chloro-1-ethanesulfonyl chloride (1.07 mL) dropwise, while cooling the flask in ice-water. The mixture was allowed to warm to room temperature once addition was complete. After 2 h triethylamine (3.6 mL) was added, followed by 2-chloro-1-ethanesulfonyl chloride (1.07 mL). After 35 min the mixture was treated with a saturated sodium bicarbonate solution (120 mL), and washed with EtOAc. The combined organic layers were dried (Na₂SO₄), filtered, and concentrated in vacuo to give a residue which was purified by flash chromatography (silica gel, 0-7% MeOH in DCM) to give the title compound as a white solid (5.58 g); ESMS m/z 1093.8 [M+H]⁺.

### b) 9-Deoxy-9a-benzyloxycarbonyl-4″-O-vinylsulfonyl-9a-aza-9a-homoerythromycin A

A stirred solution of Intermediate 51a (5.58 g) in MeOH (200 mL) was heated to 60° C. for 39.5 h. The solvent was then removed in vacuo to give a residue which was purified by flash chromatography (silica gel, 1-10% MeOH in DCM) to give the crude title compound as a white solid (2.88 g) which was used without further purification; ESMS m/z 959.6 [M+H]⁺.

### Intermediate 52: 4″-O-Vinylsulfonyl-Erythromycin A-(9E)-O-cyanomethyloxime

### a) Erythromycin A-(9E)-O-cyanomethyloxime

To a stirred solution of Erythromycin A (9E)-oxime (6.074 g) in DCM (20 mL) was added 2N aqueous NaOH (13.5 mL), tetrabutylammonium bromide, (0.302 g) and chloroacetonitrile, (0.75 mL). After 2 h the reaction was quenched by addition of brine (250 mL) and then extracted with DCM (3×250 mL). The organic extracts were combined, washed with water (250 mL), dried (Na₂SO₄) and concentrated in vacuo to give a yellow solid, which was purified by crystallisation from acetoneAwater to give the title compound as a cream solid (3.404 g); ESMS m/z 788.0 [M+H]⁺.

### b) 2″-O-Acetyl-Erythromycin A-(9E)-O-cyanomethyloxime

To a stirred solution of Intermediate 52a (4 g) in DCM (63 mL) at room temp was added acetic anhydride (0.6 mL). After 18 h, water (100 mL) and DCM (100 mL) were added and the mixture was basified to pH 9 with 2N aqueous NaOH. The organic phase was collected, dried (MgSO₄), filtered and concentrated in vacuo to give the title compound as a yellow solid (4.092 g); ESMS m/z 830.7 [M+H]⁺.

### c) 4″-O-Vinylsulfonyl-2″-O-Acetyl-Erythromycin A-(9E)-O-cyanomethyloxime

To a stirred mixture of Intermediate 52b (4.092 g) stirred under Argon at 0° C. in toluene (160 mL) were added triethylamine (2.1 mL) and 2-chloro-1-ethanesulfonyl chloride (0.77 mL). The reaction was slowly allowed to warm to rt and stirred for 2 hours, additional triethylamine (2.1 mL) and 2-chloro-1-ethanesulfonyl chloride (0.77 mL) were added. The mixture was stirred for 1 hour, and then diluted with brine (150 mL), and washed with EtOAc (3×200 mL). Combined organic phases were dried (MgSO₄), filtered, and then concentrated in vacuo and the residue purified by flash chromatography (silica gel, 0-10% [10% 0.880 aqueous ammonia in MeOH] in DCM) to give the title compound as a brown solid (2.18 g); ESMS m/z 920.6 [M+H]⁺.

### d) 4″-O-Vinylsulfonyl-Erythromycin A-(9E)-O-cyanomethyloxime

A solution of Intermediate 52c (2.18 g) in MeOH (200 mL) was stirred at 55° C. for 24 h. The mixture was concentrated in vacuo and the residue purified by flash chromatography (silica gel, 0-10% [10% 0.880 aqueous ammonia in MeOH] in DCM) to give the title compound as a white solid (1.58 g); ESMS m/z 878.5 [M+H]⁺.

### Intermediate 53: 4″-O-Vinylsulfonyl-Erythromycin A-(9E)-O-(5-methylisoxazol-3-yl)methyloxime

### a) Erythromycin A-(9E)-O-(5-methylisoxazolyl-3-)-methyloxime

To a solution of erythromycin A-(9E)-oxime, (0.3 g) in dry THF (2 mL) stirred under argon at room temperature, was added tetrabutylammonium hydroxide (1N in THF, 0.4 mL). After 10 min, 3-chloromethyl-5-methylisoxazole (0.052 mL) was added. After 18 h, the reaction was diluted with Et₂O (4 mL) and washed with water (4 mL) and brine, (4 mL). The organic phase was dried (Na₂SO₄), filtered and concentrated in vacuo to give a white foam, which was recrystallised from acetone/water to give the title compound as a white solid (0.133 g); ESMS m/z 844.7 [M+H]⁺.

### b) 211-O-Acetyl-Erythromycin A-(9E)-O-(5-methylisoxazol-3-yl)-methyloxime

Using a procedure similar to that used to prepare Intermediate 52b, Intermediate 53a (3.44 g) gave the title compound as a cream solid (3.635 g), ESMS m/z 886.7 [M+H]⁺.

### c) 4″-O-Vinylsulfonyl-2″-O-Acetyl-Erythromycin A-(9E)-O-(5-methylisoxazol-3-yl)-methyloxime

To a stirred mixture of Intermediate 53b (3.635 g) stirred under argon at 0° C. in toluene (135 mL) were added diisopropylethylamine (2.1 mL) and 2-chloro-1-ethanesulfonyl chloride (0.65 mL). The reaction was slowly allowed to warm to rt and stirred for 24 hours, at which point triethylamine (1.7 mL) and 2-chloro-1-ethanesulfonyl chloride (0.65 mL) were added. The mixture was stirred for 1 hour, and then diluted with brine (150 mL), and washed with EtOAc (3×200 mL). Combined organic phases were dried (MgSO₄), filtered, and then concentrated in vacuo and the residue purified by flash chromatography (silica gel, 0-10% [10% 0.880 aqueous ammonia in MeOH] in DCM) to give the title compound as a cream solid (4.09 g); ESMS m/z 966.6 [M+H]⁺.

### d) 4″-O-Vinylsulfonyl-Erythromycin A-(9E)-O-(5-methylisoxazol-3-yl)-methyloxime

Using a procedure similar to that used to prepare Intermediate 52, Intermediate 53c (4.09 g) gave the title compound as a white solid (3.065 g), ESMS m/z 924.7 [M+H]⁺.

### Intermediate 54: 4″-O-Vinylsulfonyl-Erythromycin A-(9E)-O—(N,N-dimethylacetamido)-oxime

### a) Erythromycin A-(9E)-O—(N,N-dimethylacetamido)-oxime

Using a procedure similar to that used to prepare Intermediate 53a, erythromycin A-(9E)-oxime (8.136 g), and N,N-dimethyl chloroacetamide (1.121 ml) gave the crude material as a cream solid, which could not be crystallised, and hence was purified by flash chromatography (silica gel, 0-10% [10% ammonia 0.880 aqueous in MeOH] in DCM) to give the title compound as a cream solid, (5.2 g), ESMS m/z 834.8 [M+H]⁺.

### b) 2″-O-Acetyl-Erythromycin A-(9E)-O-(N,N-dimethylacetamido)-oxime

Using a procedure similar to that used to prepare Intermediate 52b, Intermediate 54a (5.2 g) gave the title compound as a cream solid (5.96 g), ESMS m/z 876.8 [M+H]⁺.

### c) 4″-O-Vinylsulfonyl-2″-O-Acetyl-Erythromycin A-(9E)-O(N,N-dimethylacetamido)-oxime

Using a procedure similar to that used to prepare Intermediate 52c, Intermediate 54b (5.96 g) gave the title compound as a white solid (6.02 g), ESMS m/z 966.6 [M+H]⁺.

### d) 4″-O-Vinylsulfonyl-Erythromycin A-(9E)-O—(N,N-dimethylacetamido)-oxime

Using a procedure similar to that used to prepare Intermediate 52, Intermediate 54c gave the title compound as a white solid (5.73 g), ESMS m/z 924.7 [M+H]⁺.

### Intermediate 55: 4″-O-Vinylsuphonyl erythromycin A-(9E)-O-(2-diethylaminoethyl)-oxime

### a) 2′-O-Acetyl-erythromycin A-(9E)-O-(2-diethylaminoethyl)-oxime

To a solution of erythromycin A-(9E)-O-(2-diethylaminoethyl)-oxime (4.05 g) in DCM (50 mL) sodium hydrogen carbonate (0.6 g) was added followed by acetic anhydride (0.68 mL). After stirring overnight at room temperature the mixture was diluted with DCM and washed with 5% aqueous sodium carbonate. The organic layer was separated, dried and evaporated to yield the title product (4.1 g) as a solid; ESMS m/z 890.3 [M+H]⁺.

### b) 4″-O-Vinylsulfonyl-2′-O-acetyl-erythromycin A-(9E)-O-(2-diethylaminoethyl)-oxime

To a stirred mixture of Intermediate 55a (2.31 g) in toluene (50 mL) were added triethylamine (1.1 mL) and then dropwise 2-Chloro-1-ethane sulphonyl chloride (0.4 mL) at 20° C. under argon, and the resultant mixture was stirred for 16 hours. The mixture was concentrated and the residue purified by chromatography on silica gel eluting with 0-10% (9:1 MeOH/20M aqueous ammonia) in DCM to give crude product which was partitioned between dichrolomehane and water. The organic phase was dried over magnesium sulphate and evaporated to give title compound (2.1 g); ESMS mr/z 491.1 [M+2H]²⁺.

### c) 4″-O-Vinylsuphonyl erythromycin A (9E)-O-(2-diethylamino)ethyloxime

A solution of Intermediate 55b (2.09 g) in MeOH (60 mL) was stirred at 55° C. for 16 hours. The mixture was concentrated and the residue purified by chromatography on silica gel eluting with 0-10% (9:1 MeOH/20M aq. ammonia) in DCM to give title compound as a white solid (1.31 g); ESMS m/z 470.1 [M+2H]²⁺.

### Example 1

### 4″-O-{2-[3-(3-Carboxy-1,4-dihydro-1-ethyl-4-oxo-6-quinolinyl) propylamino]ethanesulfonyl}-6-O-methylerythromycin A

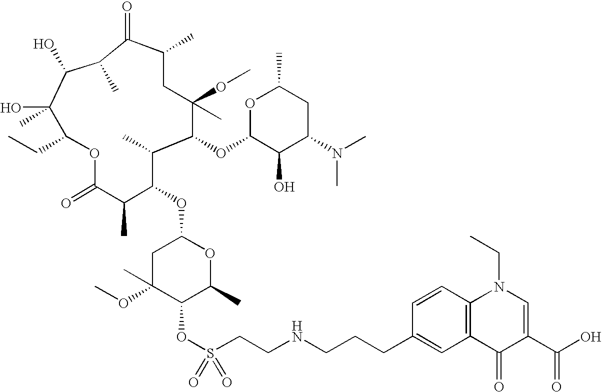

A stirred mixture of Intermediate 1 (0.084 g) and Intermediate 6 (0.078 g) in DMSO (0.5 mL) and triethylamine (0.042 mL) was heated at 40° C. After 1.5 h the mixture was cooled, diluted with MeCN, and purified by mass directed automatic preparative HPLC to give, after freeze drying from dilute aqueous ammonia, the title compound as a white solid (0.087 g); ESMS m/z 1112.9 [M+H]⁺.

### Example 2

### 4″-O-{2-[3-(3-Carboxy-1,4-dihydro-1-dimethylamino-4-oxo-6-quinolinyl)propylamino]ethanesulfonyl}-9-(S)-dihydroerythromycin A-9,11-ethylidene acetal

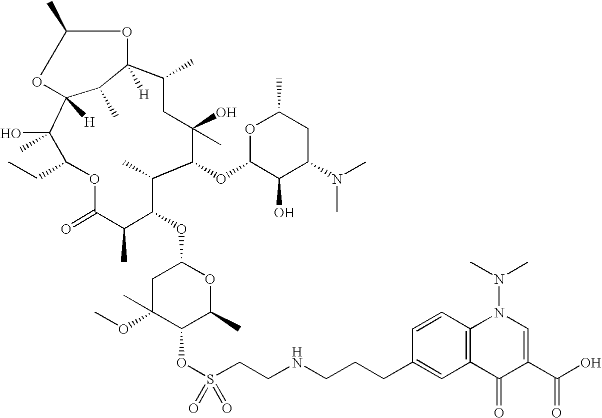

A stirred mixture of Intermediate 2 (0.112 g) and Intermediate 4 (0.073 g) in DMSO (0.75 mL) and triethylamine (0.063 mL) was heated at 50° C. After 1 h the mixture was cooled, diluted with MeCN, and purified by mass directed automatic preparative HPLC to give, after freeze drying from dilute aqueous ammonia, the title compound as a white solid (0.072 g); ESMS m/z 1142.0 [M+H]⁺.

### Example 3

### 4″-O-{2-[{2-(3-Carboxy-1,4-dihydro-1-ethyl-6-fluoro-4-oxo-[1,8]naphthyridin-7-ylamino)ethyl}amino]ethanesulfonyl}-6-O-methyl-erythromycin A formate

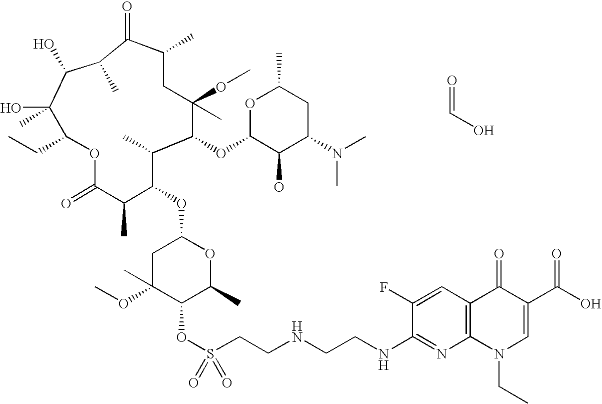

A stirred mixture of Intermediate 1 (0.126 g) and Intermediate 3 (0.092 g) in DMSO (0.75 mL) and triethylamine (0.063 mL) was heated at 40° C. for 17 h and 50° C. for 24 h. The mixture was cooled, diluted with MeCN, and purified by mass directed automatic preparative HPLC to give, after freeze drying from dilute aqueous ammonia, the title compound as a white solid (0.123 g); ESMS m/z 1132.9 [M+H]⁺.

### Example 4

### 4″-O-{2-[2-(3-Carboxy-1,4-dihydro-1-ethyl-4-oxo-[1,7]naphthyridin-6-ylsulfanyl)-ethylamino]ethanesulfonyl}-6-O-methyl-erythromycin A

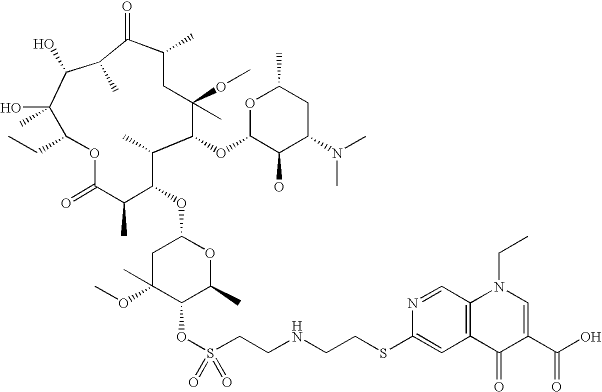

A stirred mixture of Intermediate 1 (0.126 g) and Intermediate 9 (0.092 g) in DMSO (0.75 mL) and triethylamine (0.063 mL) was heated at 40° C. for 17 h. The mixture was cooled, diluted with MeCN, and purified by mass directed automatic preparative HPLC to give, after freeze drying from dilute aqueous ammonia, the title compound as a cream solid (0.125 g); ESMS m/z 1131.8 [M+H]⁺.

### Example 5

### 4″-O-{2-[3-(3-Carboxy-1,4-dihydro-1-dimethylamino-4-oxo-6-quinolinyl)propylamino]ethanesulfonyl}-6-O-methyl-erythromycin A

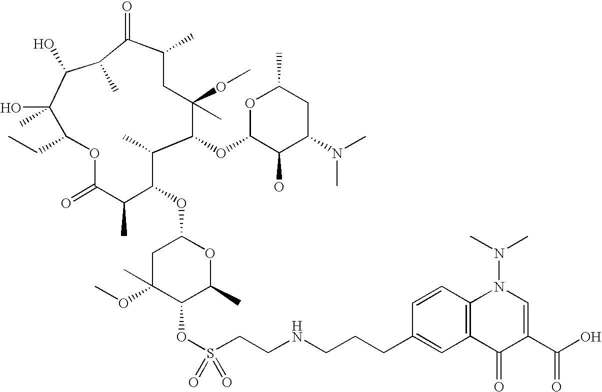

A stirred mixture of Intermediate 1 (0.126 g) and Intermediate 4 (0.073 g) in DMSO (0.75 mL) and triethylamine (0.063 mL) was heated at 40° C. for 17 h and 50° C. for 75 min. The mixture was cooled, diluted with MeCN, and purified by mass directed automatic preparative HPLC to give, after freeze drying from dilute aqueous ammonia, the title compound as a white solid (0.137 g); ESMS m/z 1127.9 [M+H]⁺.

### Example 6

### 4″-O-{2-[3-(3-Carboxy-1,4-dihydro-1-ethyl 4-oxo-[1,8]naphthyridin-6-yl)propylamino]ethanesulfonyl}-6-O-methyl-erythromycin A

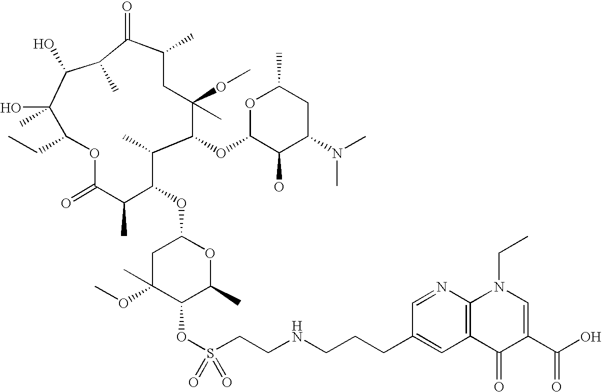

A stirred mixture of Intermediate 1 (0.126 g) and Intermediate 5 (0.088 g) in DMSO (0.75 mL) and triethylamine (0.063 mL) was heated at 40° C. for 17 h and 50° C. for 1.5 h. The mixture was cooled, diluted with MeCN, and purified by mass directed automatic preparative HPLC to give, after freeze drying from dilute aqueous ammonia, the title compound as a white solid (0.127 g); ESMS m/z 1113.9 [M+H]⁺.

### Example 7

### 4″-O-{2-[2-(3-Carboxy-1,4-dihydro-1-Dimethylamino-4-oxo-[1,7]naphthyridin-6-ylsulfanyl)-ethylamino]ethanesulfonyl}-6-O-methyl-erythromycin A formate

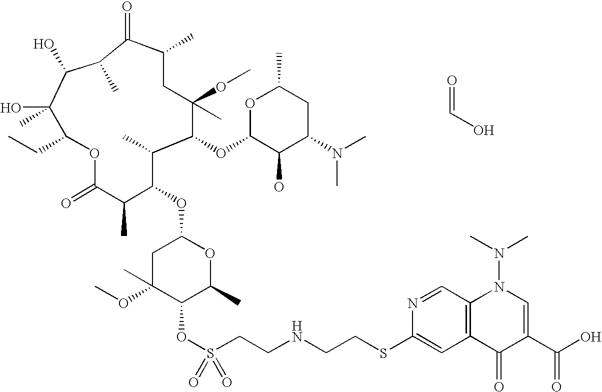

A stirred mixture of Intermediate 1 (0.126 g) and Intermediate 10 (0.078 g) in DMSO (0.75 mL) and triethylamine (0.063 mL) was heated at 50° C. for 17 h. The mixture was cooled, diluted with MeCN, and purified by mass directed automatic preparative HPLC to give, after freeze drying from dilute aqueous ammonia, the title compound as a cream/yellow solid (0.104 g); ESMS m/z 1146.9 [M+H]⁺.

### Example 8

### 4″-O-{2-[3-(6-Carboxy-7-oxo-2,3-dihydro-1H,7H-pyrido[3,2,1-ij]quinolin-9-yl)propylamino]ethanesulfonyl}6-O-methylerythromycin A formate

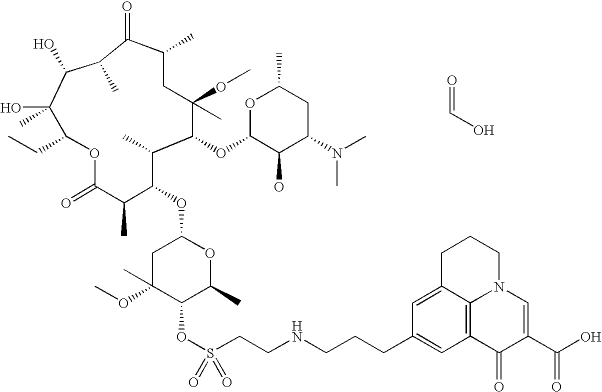

A stirred mixture of Intermediate 1 (0.126 g) and Intermediate 7 (0.121 g) in DMSO (0.75 mL) and triethylamine (0.063 mL) was heated at 50° C. for 17 h. The mixture was cooled, diluted with MeCN, and purified by mass directed automatic preparative HPLC to give, after freeze drying from dilute aqueous ammonia, the title compound as a white solid (0.145 g); ESMS m/z 1124.9 [M+H]⁺.

### Example 9

### 4″-O-{[(2-{[3-(6-Carboxy-3-methyl-7-oxo-1H,7H-[1,3]oxazino[5,4,3-ij]quinolin-9-yl)propyl]amino}ethyl)sulfonyl]-6-O-methylerythromycin A formate

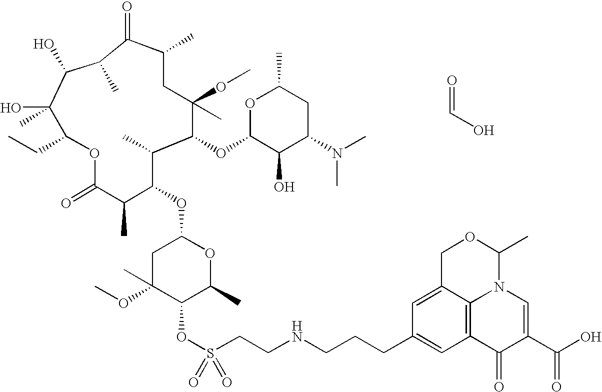

A stirred mixture of Intermediate 1 (0.126 g) and Intermediate 11 (0.082 g) in DMSO (0.75 mL) and triethylamine (0.063 mL) was heated at 50° C. for 3.5 h. The mixture was cooled, diluted with MeCN, and purified by mass directed automatic preparative HPLC to give, after freeze drying from dilute aqueous ammonia, the title compound as a white solid (0.142 g); ESMS m/z 1140.9 [M+H]⁺.

### Example 10

### 4″-O-{2-[3-(3-Carboxy-1,4-dihydro-1-(2,2,2-trifluoroethyl)-4-oxo-6-quinolinyl)propylamino]ethanesulfonyl}-6-O-methylerythromycin A

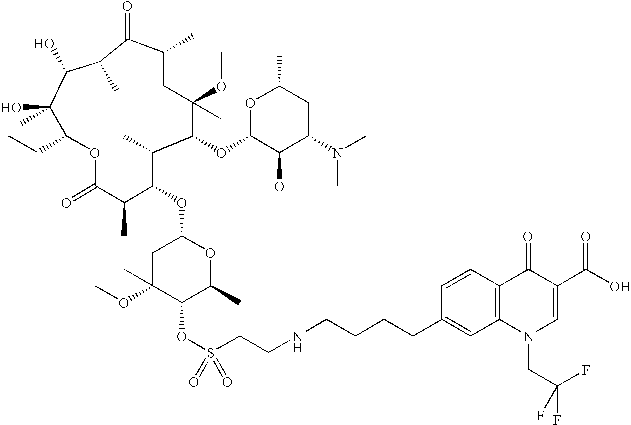

A stirred mixture of Intermediate 1 (0.126 g) and Intermediate 8 (0.099 g) in DMSO (0.75 mL) and triethylamine (0.063 mL) was heated at 50° C. for 5 h. The mixture was cooled, diluted with MeCN, and purified by mass directed automatic preparative HPLC to give, after freeze drying from dilute aqueous ammonia, the title compound as a white solid (0.117 g); ESMS m/z 1166.8 [M+H]⁺.

### Example 11

### 4″-O-{2-[2-(3-Carboxy-1,4-dihydro-1-dimethylamino-4-oxo-6-quinolinyl)sulfanylethylamino]ethanesulfonyl}-6-O-methylerythromycin A

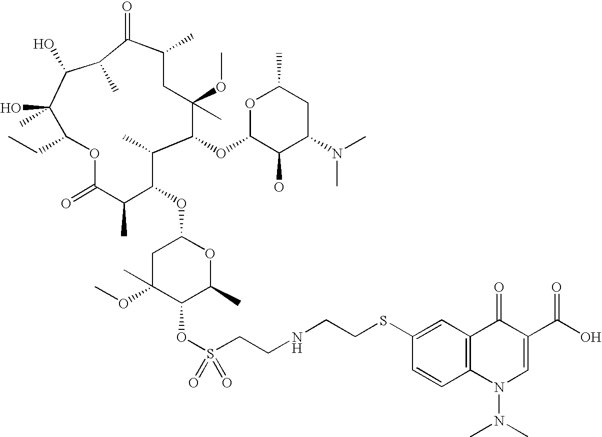

A stirred mixture of Intermediate 1 (0.126 g) and Intermediate 13 (0.095 g) in DMSO (0.75 mL) and triethylamine (0.063 mL) was heated at 50° C. for 5 h. The mixture was cooled, diluted with MeCN, and purified by mass directed automatic preparative HPLC to give, after freeze drying from dilute aqueous ammonia, the title compound as a white solid (0.102 g); ESMS m/z 1145.9 [M+H]⁺.

### Example 12

### 4″-O-[(2-{[3-(7-Carboxy-8-oxo-3,4-dihydro-2H,8H-[1,4]oxazepino[2,3,4-ij]quinolin-10-yl)propyl]amino}ethyl)sulfonyl]-6-O-methylerythromycin A

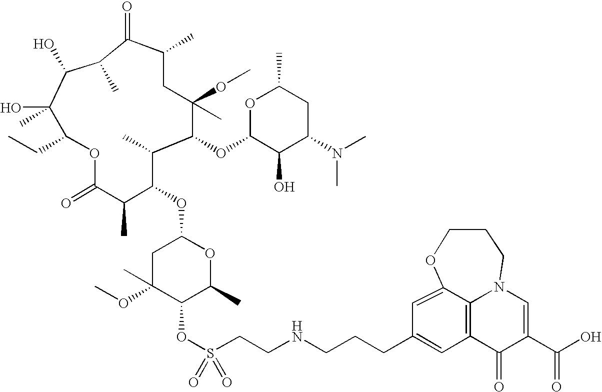

A stirred mixture of Intermediate 14 (0.068 g) and Intermediate 1 (0.126 g) in DMSO (1.5 mL) and triethylamine (0.20 mL) was heated at 50° C. After 3 h the mixture was cooled and purified by mass directed automatic preparative HPLC followed by chromatography (silica gel, 0 to 15% [9:1 MeOH/20 M aq. ammonia] in DCM) to give the title compound as a white solid (0.087 g); ESMS m/z 1141.0 [M+H]⁺.

### Example 13

### 4″-O-[(2-{[3-(6-Carboxy-3,3-dimethyl-7-oxo-1H,7H-[1,3]oxazino[5,4,3-ij]quinolin-9-yl)propyl]amino}ethyl)sulfonyl]-6-O-methylerythromycin A

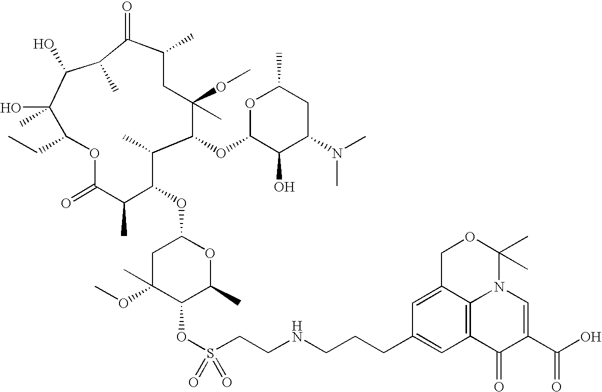

A stirred mixture of Intermediate 15 (0.097 g) and Intermediate 1 (0.126 g) in DMSO (1.5 mL) and triethylamine (0.20 mL) was heated at 50° C. After 2.5 h the mixture was cooled and purified by mass directed automatic preparative HPLC followed by chromatography (silica gel, 0 to 15% [9:1 MeOH/20 M aq. ammonia] In DCM) to give the title compound as a white solid (0.069 g); ESMS m/z 1155.0 [M+H]⁺.

### Example 14

### 4″-O-{[2-({2-[(2-Carboxy-5-methyl-1-oxo-6,7-dihydro-1H,5H-pyrido[3,2,1-ij]quinolin-9-yl)thio]ethyl}amino)ethyl]sulfonyl}-6-O-methylerythromycin A

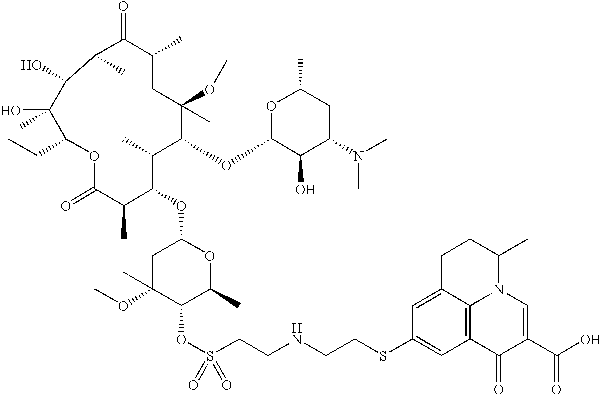

A stirred mixture of Intermediate 16 (0.065 g) and Intermediate 1 (0.1 g) in DMSO (1 mL) and triethylamine (0.15 mL) was heated at 50° C. After 2 h the mixture was cooled and purified by mass directed automatic preparative HPLC followed by chromatography (silica gel, 0 to 15% [9:1 MeOH/20 M aq. ammonia] in DCM) to give the title compound as a white solid (0.1 g); ESMS m/z 1156.9 [M+H]⁺.

### Example 15

### 4″-O-(2-[(2-{[6-carboxy-8-dimethylamino-5-oxo-5,8-dihydro-[1,8]naphthyridin-3-yl]thio}ethyl)amino]ethyl}sulfonyl)-6-O-methyl-erythromycin A formate

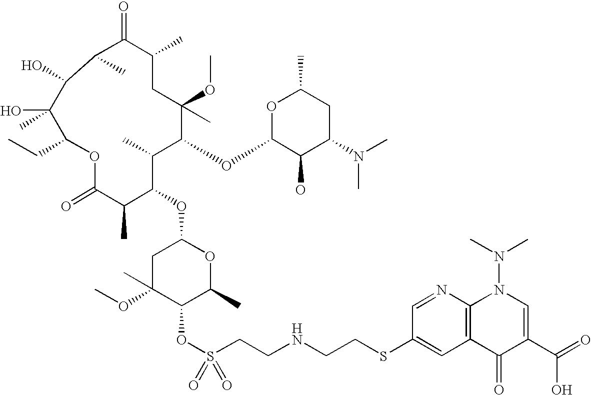

A stirred mixture of Intermediate 1 (0.125 g) and Intermediate 17 (0.126 g) in DMSO (1 mL), water (1 drop) and triethylamine (0.084 mL) was heated at 50° C. After 24 hours the mixture was cooled and filtered. The filtrate was purified by mass directed automatic preparative HPLC and then flash chromatography (silica gel, 0-15% (10% v/v 20 M aq. ammonia in MeOH) In DCM) to give the title compound as a white solid (0.052 g); ESMS m/z 1147.0 [M+H]⁺.

### Example 16

### 4″-O-{2-({3-[6-Carboxy-3,3-dimethyl-7-oxo-1H,7H-[1,3]oxazino[5,4,3-ij]quinolin-9-yl]propyl}amino)ethanesulfonyl}-erythromycin A-(9E)-oxime

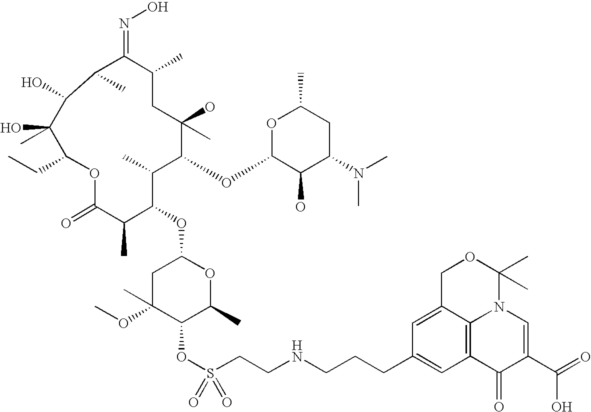

A stirred mixture of Intermediate 18 (0.15 g) and Intermediate 15 (0.115 g) in DMSO (2.5 mL) and triethylamine (0.4 mL) was heated at 50° C. After 4 hours the mixture was cooled and then concentrated. The residue was purified by mass directed automatic preparative HPLC and then flash chromatography (silica gel, 0-15% (10% 20 M aq. ammonia in MeOH) in DCM) to give the title compound as a white solid (0.128 g); ESMS m/z 1156.0 [M+H]⁺.

### Example 17

### 4″-O-{[2-({2-[(2-Carboxy-5-methyl-1-oxo-6,7-dihydro-1H,5H-pyrido[3,2,1-ij]quinolin-9-yl)thio]ethyl}amino)ethyl]sulfonyl}erythromycin A-(9E)-oxime

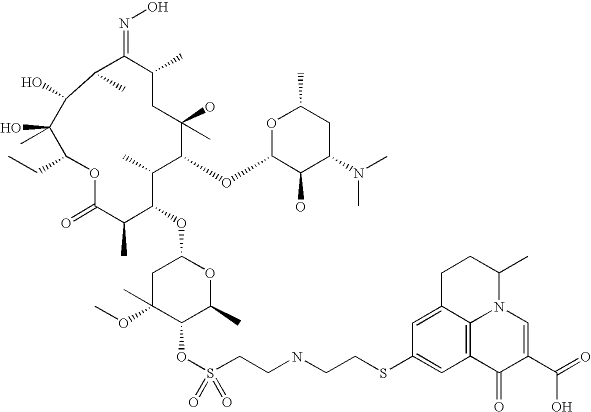

A stirred mixture of Intermediate 18 (0.15 g) and Intermediate 16 (0.116 g) in DMSO (2.5 mL) and triethylamine (0.4 mL) was heated at 50° C. After 4 h the mixture was cooled and then concentrated. The residue was purified by mass directed automatic preparative HPLC and then flash chromatography (silica gel, 0-15% (10% 20M aq. ammonia in MeOH) in DCM) to give the title compound as a white solid (0.103 g); ESMS m/z 1158.0 [M+H]⁺.

### Example 18

### 4″-O-{2-[3-(3-Carboxy-1,4-dihydro-1-(2-fluoroethyl)-4-oxo-6-quinolinyl)propylamino]ethanesulfonyl}-6-O-methylerythromycin A

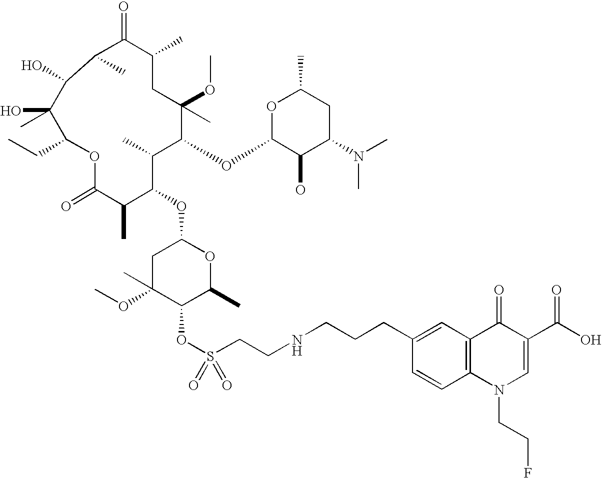

A stirred mixture of Intermediate 1 (0.126 g) and Intermediate 19 (0.091 g) in DMSO (0.75 mL) and triethylamine (0.063 mL) was heated at 50° C. for 1.5 h. The mixture was cooled, diluted with MeCN, and purified by mass directed automatic preparative HPLC to give, after freeze drying from dilute aqueous ammonia, the title compound as a white solid (0.116 g); ESMS m/z 1131.0 [M+H]⁺.

### Example 19

### 4″-O-{2-({3-[3-Carboxy-1-ethyl-4-oxo-1,4-dihydro-6-quinolinyl]propyl}amino)ethanesulfonyl}-erythromycin A-(9E)-oxime

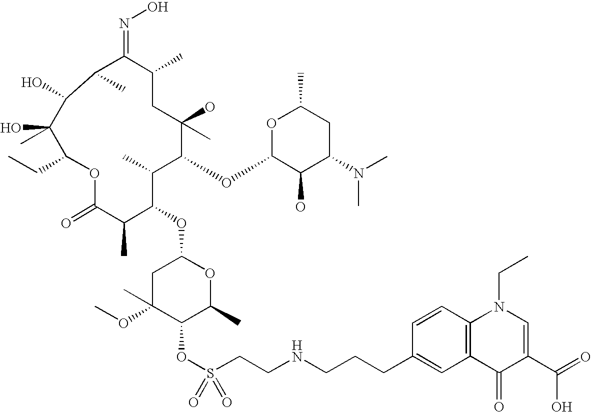

A stirred mixture of Intermediate 18 (0.15 g) and Intermediate 6 (0.104 g) in DMSO (2.5 mL) and triethylamine (0.4 mL) was heated at 50° C. After 4 hours the mixture was cooled and then concentrated. The residue was purified by mass directed automatic preparative HPLC and then flash chromatography (silica gel, 0-15% (10% 20 M aq. ammonia in MeOH) in DCM) to give the title compound as a white solid (0.128 g); ESMS m/z 1113.8 [M+H]⁺.

### Example 20

### 4″-O-{2-[3-(3-Carboxy-1,4-dihydro-1-dimethylamino-4-oxo-6-quinolinyl)propylamino]ethanesulfonyl}-azithromycin-11,12-carbonate

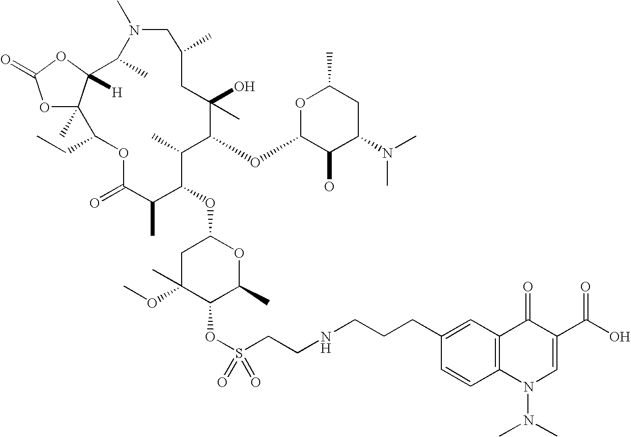

A stirred mixture of Intermediate 20 (0.123 g) and Intermediate 4 (0.069 g) in DMSO (0.75 mL) and triethylamine (0.059 mL) was heated at 50° C. for 1.5 h. The mixture was cooled, diluted with MeCN, and purified by mass directed automatic preparative HPLC to give, after freeze drying from dilute aqueous ammonia, the title compound as a white solid (0.099 g); ESMS m/z 1155.0 [M+H]⁺.

### Example 21

### 4″-O-{2-[3-(3-Carboxy-1,4-dihydro-1-ethyl-4-oxo-6-quinolinyl) propylamino]ethanesulfonyl}-azithromycin-11,12-carbonate

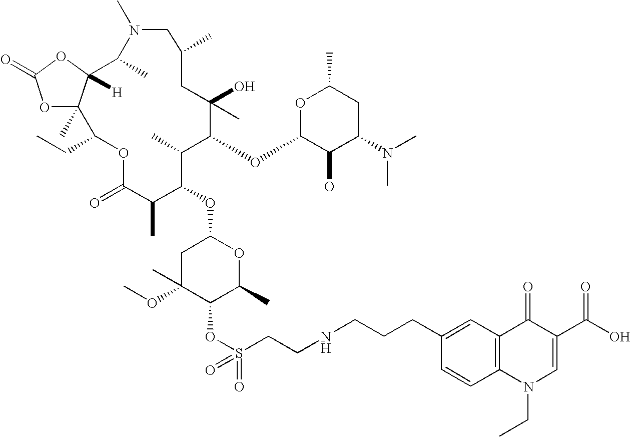

A stirred mixture of Intermediate 20 (0.127 g) and Intermediate 6 (0.085 g) in DMSO (0.75 mL) and triethylamine (0.061 mL) was heated at 50° C. for 2 h. The mixture was cooled, diluted with MeCN, and purified by mass directed automatic preparative HPLC to give, after freeze drying from dilute aqueous ammonia, the title compound as a white solid (0.092 g); ESMS m/z 1140.0 [M+H]⁺.

### Example 22

### 4″-O-{2-[3-(3-Carboxy-1,4-dihydro-1-ethyl-4-oxo-6-quinolinyl) propyloxy]ethanesulfonyl}-6-O-methylerythromycin A

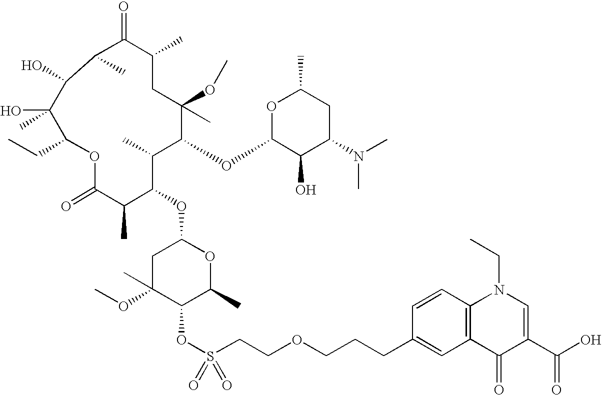

A stirred mixture of Intermediate 1 (0.084 g) and Intermediate 21 (0.042 g) in DMSO (0.5 mL) and potassium carbonate (0.028 g) was heated at 80° C. After 24 h the mixture was purified by mass directed automatic preparative HPLC and then flash chromaotraphy (silica gel, 10-20% [9:1 MeOH:20M ammonia] in DCM) to give the title compound as a white solid (0.01 g); ESMS m/z 1114.1 [M+H]⁺.

### Example 23

### 4″-O-{2-({3-[3-Carboxy-1-ethyl-4-oxo-1,4-dihydro-6-quinolinyl]propyl}amino)ethanesulfonyl}erythromycin A-(9E)-O-methoxymethyloxime

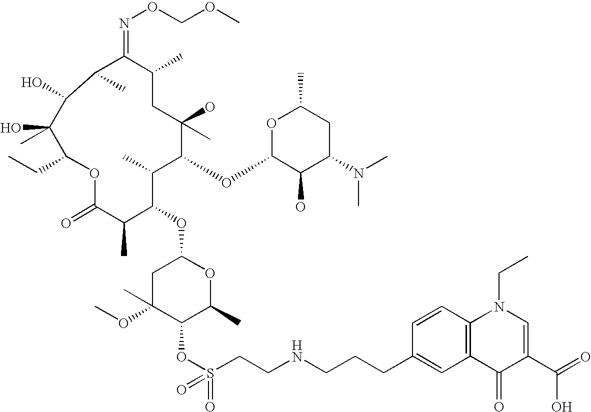

A stirred mixture of Intermediate 22 (0.06 g) and Intermediate 6 (0.04 g) in DMSO (2 mL) and triethylamine (0.3 mL) was heated at 50° C. After 3 hours the mixture was cooled and then concentrated. The residue was purified by mass directed automatic preparative HPLC and then flash chromatography (silica gel, 0-15% (10% 20 M aq. ammonia in MeOH) in DCM) to give the title compound as a white solid (0.035 g); ESMS m/z 1158.0 [M+H]⁺.

### Example 24

### 4″-O-{2-[3-(3-Carboxy-1,4-dihydro-1-Dimethylamino-4-oxo-6-quinolinyl)propylamino]ethanesulfonyl}-erythromycin A-(9E)-oxime-11,12-carbonate

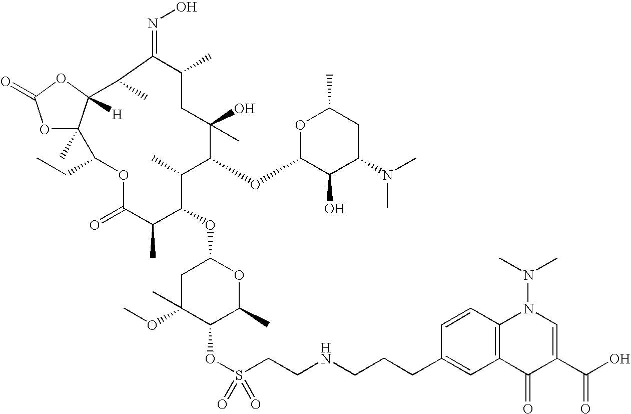

A stirred mixture of Intermediate 23 (0.13 g) and Intermediate 4 (0.073 g) in DMSO (0.75 mL) and triethylamine (0.063 mL) was heated at 50° C. for 4 h. The mixture was cooled, diluted with MeCN, and purified by mass directed automatic preparative HPLC to give, after freeze drying from dilute aqueous ammonia, the title compound as a white solid (0.109 g); ESMS m/z 1155.0 [M+H]⁺.

### Example 25

### 4″-O-{2-[3-(3-Carboxy-1,4-dihydro-1-ethyl-4-oxo-6-quinolinyl) propylamino]ethanesulfonyl}-erythromycin A-(9E)-oxime-11,12-carbonate

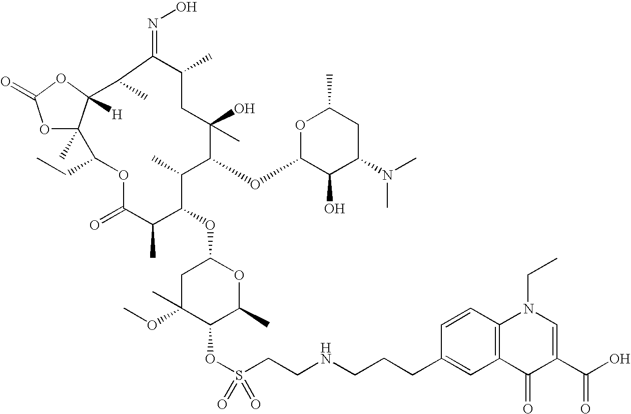

A stirred mixture of Intermediate 23 (0.13 g) and Intermediate 6 (0.087 g) in DMSO (0.75 mL) and triethylamine (0.063 mL) was heated at 50° C. for 8.5 h. The mixture was cooled, diluted with MeCN, and purified by mass directed automatic preparative HPLC to give, after freeze drying from dilute aqueous ammonia, the title compound as a white solid (0.106 g); ESMS m/z 1140.0 [M+H]⁺.

### Example 26

### 4″-O-{2-[2-(3-Carboxy-1,4-dihydro-1-ethyl-6-fluoro-4-oxo-[1,8]naphthyridin-7-ylamino)ethylamino]ethanesulfonyl}-erythromycin A-(9E)-oxime-11,12-carbonate

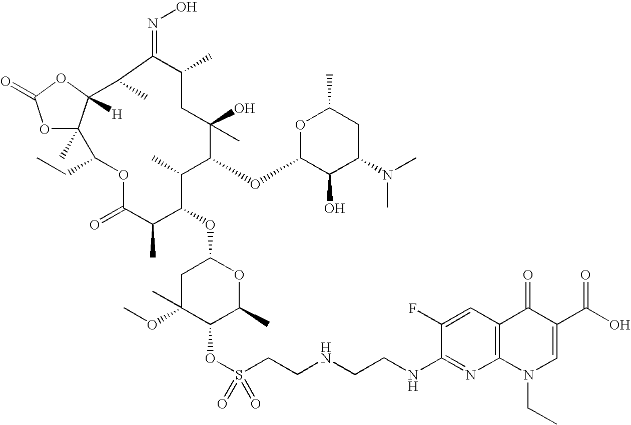

A stirred mixture of Intermediate 23 (0.13 g) and Intermediate 3 (0.092 g) in DMSO (0.75 mL) and triethylamine (0.063 mL) was heated at 50° C. for 30 h. The mixture was cooled, diluted with MeCN, and purified by mass directed automatic preparative HPLC to give, after freeze drying from dilute aqueous ammonia, the title compound as a white solid (0.098 g); ESMS m/z 1160.0 [M+H]⁺.

### Example 27

### 4″-O-{2-[3-(3-Carboxy-1,4-dihydro-1-(morpholin-4-yl)-4-oxo-6-quinolinyl)propylamino]ethanesulfonyl}-6-O-methylerythromycin A

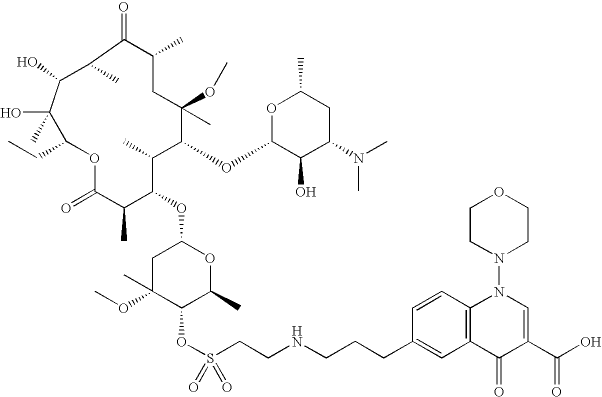

A stirred mixture of Intermediate 1 (0.084 g) and Intermediate 12 (0.055 g) in DMSO (0.5 mL) and triethylamine (0.04 mL) was heated at 50° C. After 4 h the mixture was cooled, diluted with DCM, filtered through celite, washed with saturated sodium hydrogen carbonate solution and water, dried (Na₂SO₄) and flash chromatographed (silica gel, 8-16% [9:1 MeOH:20M ammonia] in DCM) to give the title compound as a white solid (0.071 g); ESMS m/z 1169.9 [M+H]⁺.

### Example 28

### 4″-O-{2-[3-(3-Carboxy-1,4-dihydro-1-dimethylamino-4-oxo-7-quinolinyl)oxyethylamino]ethanesulfonyl}-6-O-methylerythromycin A

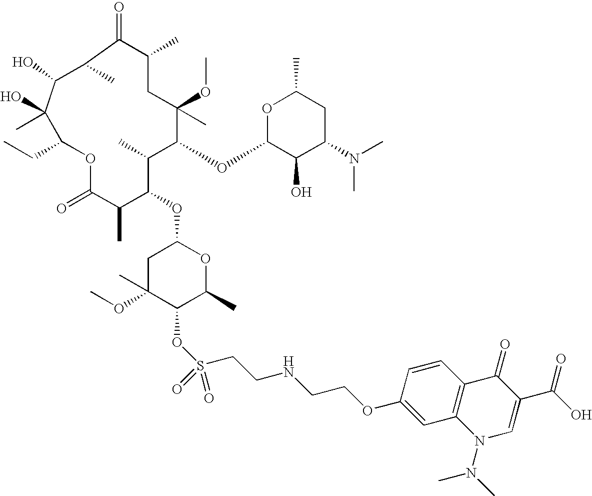

A stirred mixture of Intermediate 1 (0.084 g) and Intermediate 25 (0.049 g) in DMSO (0.5 mL) and triethylamine (0.04 mL) was heated at 50° C. After 4 h the mixture was cooled, diluted with DCM, filtered through celite, washed with saturated sodium hydrogen carbonate solution and water, dried (Na₂SO₄) and flash chromatographed (silica gel, 8-16% [9:1 MeOH:20M aq. ammonia] in DCM) to give the title compound as a white solid (0.067 g); ESMS m/z 1130.0 [M+H]⁺.

### Example 29

### 4″-O-{2-[(3R)-3-({3-[3-carboxy-1-ethyl-4-oxo-1,4-dihydro-6-quinolinyl]propyloxy}oxy)-1-pyrrolidinyl]ethanesulfonyl}-erythromycin A-(9E)-oxime

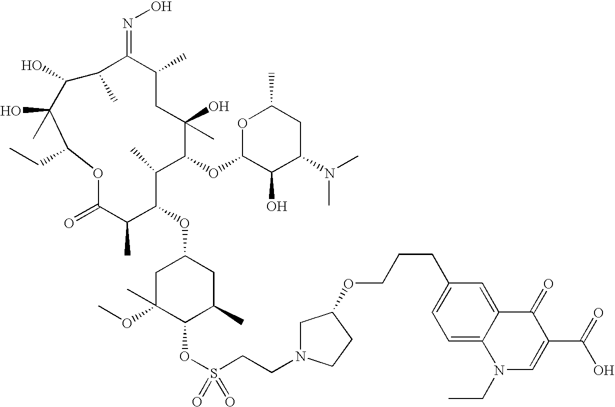

A stirred mixture of Intermediate 23 (0.15 g) and Intermediate 24 (0.158 g) in DMSO (2.5 mL) and triethylamine (0.4 mL) was heated at 50° C. After 4 hours the mixture was cooled and then concentrated. The residue was purified by mass directed automatic preparative HPLC and then flash chromatography (silica gel, 0-15% (10% 20 M aq. ammonia in MeOH) in DCM) to give the title compound as a white solid (0.073 g); ESMS m/z 1183 [M+H]⁺.

### Example 30

### 4″-O-{2-[3-(3-Carboxy-1,4-dihydro-1-dimethylamino-4-oxo-6-quinolinyl)propylamino]ethanesulfonyl}-azithromycin diformate salt

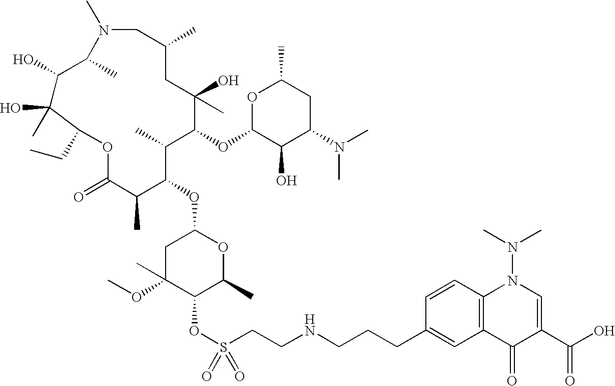

A stirred mixture of Intermediate 26 (0.084 g) and Intermediate 4 (0.065 g) in DMSO (0.5 mL) and triethylamine (0.056 mL) was heated at 50° C. for 3 h. The mixture was cooled, diluted with MeCN, and purified by mass directed automatic preparative HPLC to give, after freeze drying from dilute aqueous ammonia, the title compound as a white solid (0.064 g); ESMS m/z 1129.1 [M+H]⁺.

### Example 31

### 4″-O-{2-[3-(3-Carboxy-1,4-dihydro-1-dimethylamino-4-oxo-6-quinolyl)propylamino]ethanesulfonyl}-erythromycin A-11,12-carbonate

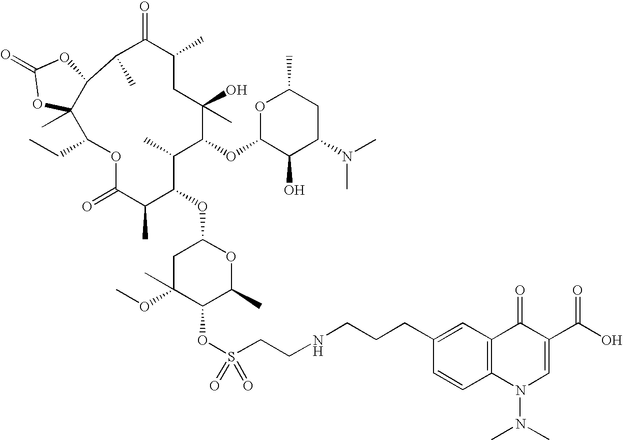

A stirred mixture of Intermediate 27 (0.149 g) and Intermediate 4 (0.085 g) in DMSO (0.9 mL) and triethylamine (0.073 mL), was heated at 50° C. for 2 h. The mixture was cooled, diluted with MeCN, and purified by mass directed automatic preparative HPLC to give, after freeze drying from dilute aqueous ammonia, the title compound as a white solid (0.131 g); ESMS m/z 1140.0 [M+H]⁺.

### Example 32

### 4″-O-{2-[3-(3-Carboxy-1,4-dihydro-1-dimethylamino-4-oxo-7-quinolinyl)oxypropylamino]ethanesulfonyl}-6-O-methylerythromycin A

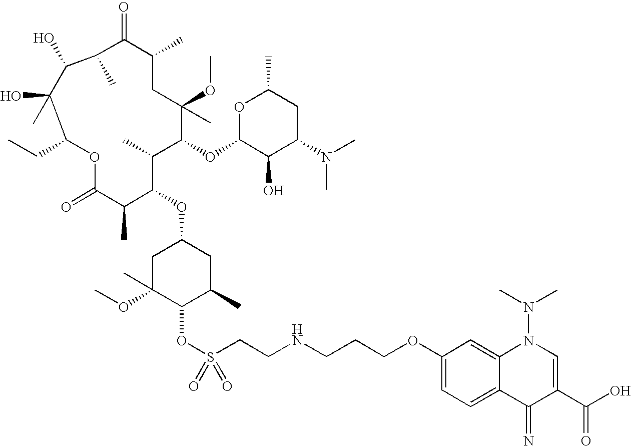

A stirred mixture of Intermediate 1 (0.084 g) and Intermediate 29 (0.051 g) in DMSO (0.5 mL), triethylamine (0.040 mL) and water (1 drop) was heated at 60° C. After 4 h the mixture was cooled, diluted with DCM and washed with saturated aq. sodium hydrogen carbonate and water. After drying (Na₂SO₄) and evaporation under reduced pressure, the residue was purified by chromatography (silica gel, 0-15% (10% 20 M aq. ammonia in MeOH) in DCM) mass directed automatic preparative HPLC and then flash chromatography (silica gel, 8-16% (10% 20 M aq. ammonia in MeOH) in DCM) followed by mass directed automatic preparative HPLC to give, after freeze drying from dilute aqueous ammonia, the title compound as a white solid (0.048 g); ESMS m/z 1143.09 [M+H]⁺.

### Example 33

### 4″-O-{2-[3-(3-Carboxy-1,4-dihydro-1-dimethylamino-4-oxo-quinolinyl)propyloxy]ethanesulfonyl}-6-O-methylerythromycin A

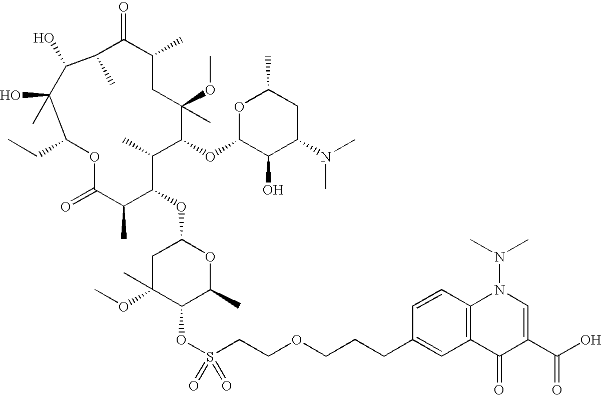

A stirred mixture of Intermediate 1 (0.084 g) and Intermediate 30 (0.087 g) in DMSO (0.5 mL) and potassium carbonate (0.048 g) was heated at 80° C. After 24 h the mixture was purified by mass directed automatic preparative HPLC and then flash chromaotraphy (silica gel, 10-20% [9:1 MeOH: 20 M aq. ammonia] In DCM) to give the title compound as a white solid (0.022 g); ESMS m/z 1129.0 [M+H]⁺.

### Example 34

### 4″-O-{2-[3-(3-Carboxy-1,4-dihydro-1-amino-4-oxo-7-quinolinyl)propylamino]ethanesulfonyl}-6-O-methylerythromycin A

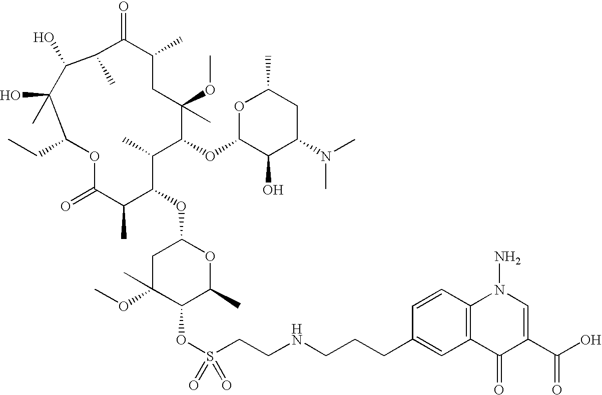

A stirred mixture of Intermediate 1 (0.034 g) and Intermediate 31 (0.019 g) in DMSO (0.3 mL), triethylamine (0.020 mL) and water (1 drop) was heated at 80° C. After 4 h the mixture was cooled, diluted with DCM and washed with saturated aq. sodium hydrogen carbonate and water. After drying (MgSO₄) and evaporation under reduced pressure, the residue was purified by chromatography (silica gel, 10-20% (10% 20 M aq. ammonia in MeOH) in DCM) to give the title compound as a white solid (0.012 g); ESMS m/z 1100.0[M+H]⁺.

### Example 35

### 4″-O-{[2-({2-[(2-([2-Carboxy-5-methyl-1-oxo-6,7-dihydro-1H,5H-pyrido[3,2,1-ij]quinolin-9-yl]amino}ethyl)oxy]ethyl}oxy)ethyl]sulfonyl}-6-O-methylerythromycin A

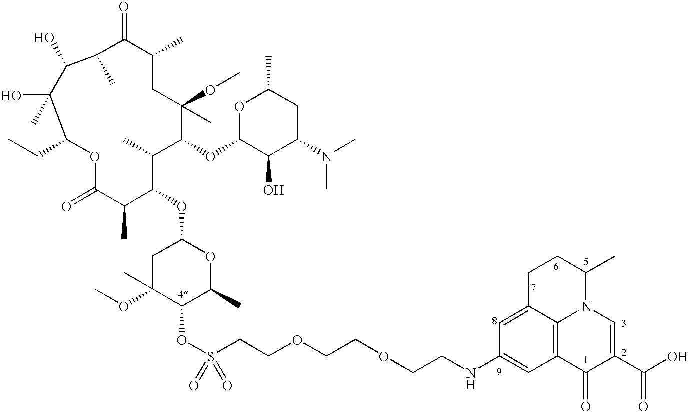

A stirred mixture of Intermediate 32 (0.07 g), Intermediate 1 (0.392 g) and potassium carbonate (0.035 g) in DMSO (0.5 mL) was heated at 70° C. overnight. The reaction mixture was concentrated and purified by chromatography (silica gel, 0 to 15% [9:1 MeOH/20 M aq. ammonia] in DCM to give the title compound as a pale yellow solid (0.07 g); ESMS m/z 1185.1 [M+H]⁺.

### Example 36

### 4″-O-{[2-({2-[(2-{[2-Carboxy-5-methyl-1-oxo-6,7-dihydro-1H,5H-pyrido[3,2,1-ij]quinolin-9-yl]amino}ethyl)oxy]ethyl}amino)ethyl]sulfonyl}-6-O-methylerythromycin A

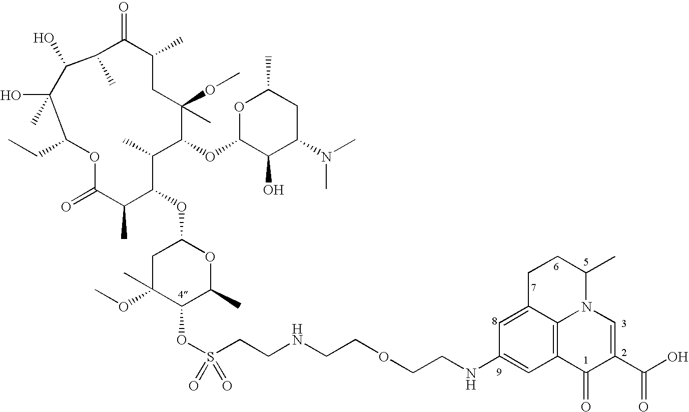

A stirred mixture of Intermediate 33 (0.07 g) and Intermediate 1 (0.126 g) in DMSO (1 mL) and triethylamine (0.15 mL) was heated at 50° C. After 2 h the mixture was concentrated and purified by chromatography (silica gel, 0 to 15% [9:1 MeOH/20 M aq. ammonia] in DCM to give the title compound as a pale yellow solid (0.11 g); ESMS m/z 1184.1 [M+H]⁺.

### Example 37

### 4″-O-[(2-{[3-(6-Carboxy-3,3-dimethyl-7-oxo-2,3-dihydro-7H-[1,4]oxazino[2,3,4-ij]quinolin-9-yl)propyl]amino}ethyl)sulfonyl]-6-O-methyl erythromycin A

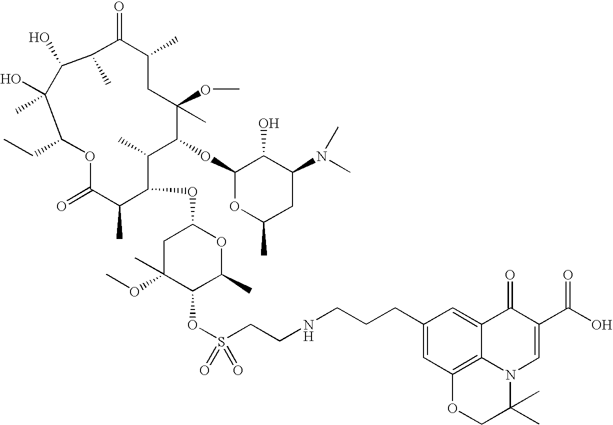

A stirred mixture of Intermediate 1 (0.126 g) and Intermediate 34 (0.076 g) in DMSO (1 mL) and triethylamine (0.2 mL) was heated at 50° C. After 1 h the mixture was concentrated and purified by chromatography (silica gel, 0 to 15% [9:1 MeOH/20 M aq. ammonia] in DCM to give the title compound as a white solid (0.095 g); ESMS m/z 1155.1 [M+H]⁺.

### Example 38

### 4″-O-{2-[3-(3-Carboxy-1,4-dihydro-1-ethyl-4-oxo-6-quinolinyl)propylamino]ethanesulfonyl}-erythromycin A-11,12-carbonate

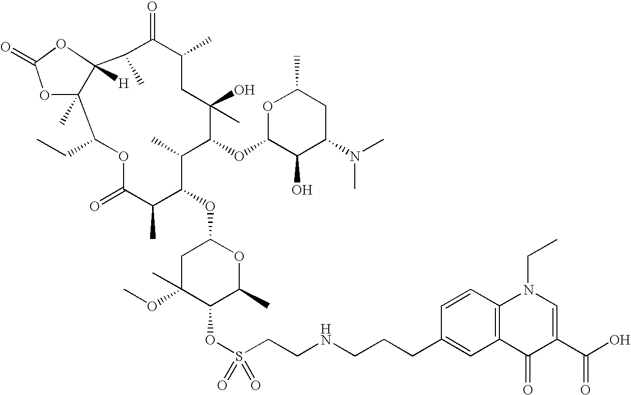

A stirred mixture of Intermediate 27 (0.149 g) and Intermediate 6 (0.102 g) in DMSO (0.9 mL) and triethylamine (0.073 mL) was heated at 50° C. for 2 h. The mixture was cooled, diluted with MeCN, and purified by mass directed automatic preparative HPLC to give, after freeze drying from dilute aqueous ammonia, the title compound as a white solid (0.098 g); ESMS m/z 1125.0 [M+H]⁺.

### Example 39

### 4″-O-{2-[2-(3-Carboxy-1,4-dihydro-1-ethyl-6-fluoro-4-oxo-[1,8]naphthyridin-7-ylamino)ethylamino]ethanesulfonyl}-erythromycin A-11,12-carbonate

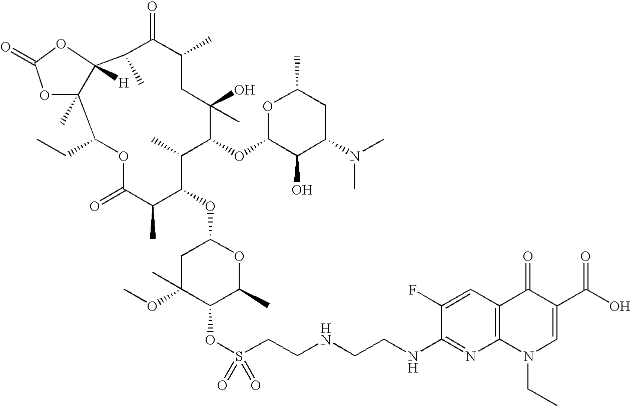

A stirred mixture of Intermediate 27 (0.149 g) and Intermediate 3 (0.143 g) in DMSO (0.9 mL) and triethylamine (0.15 mL) was heated at 50° C. for 25 h. The mixture was cooled, diluted with MeCN, and purified by mass directed automatic preparative HPLC to give, after freeze drying from dilute aqueous ammonia, the title compound as a white solid (0.071 g); ESMS m/z 1145.1 [M+H]⁺.

### Example 40

### 4″-O-{2-[3-(3-Carboxy-1,4-dihydro-1-ethyl-4-oxo-6-quinolinyl)propylamino]ethanesulfonyl}-9-(S)-dihydroerythromycin A-9,11-ethylidene acetal

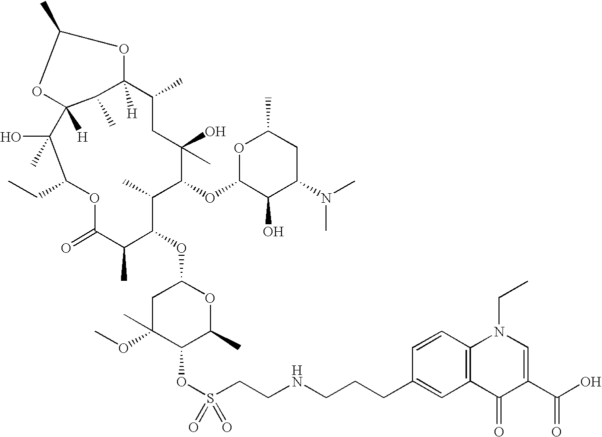

A stirred mixture of Intermediate 2 (0.087 g) and Intermediate 6 (0.058 g) in DMSO (0.75 mL) and triethylamine (0.042 mL) was heated at 50° C. for 18 h. The mixture was cooled, diluted with MeCN, and purified by mass directed automatic preparative HPLC to give, after freeze drying from dilute aqueous ammonia, the title compound as a white solid (0.089 g); ESMS m/z 1127.1 [M+H]⁺.

### Example 41

### 4″-O-{2-[3-(6-Carboxy-7-oxo-1H,7H-[1,3]oxazino[5,4,3-ij]quinolin-9-yl)propylamino]ethanesulfonyl}-erythromycin A-(9E)-O-methoxymethyloxime

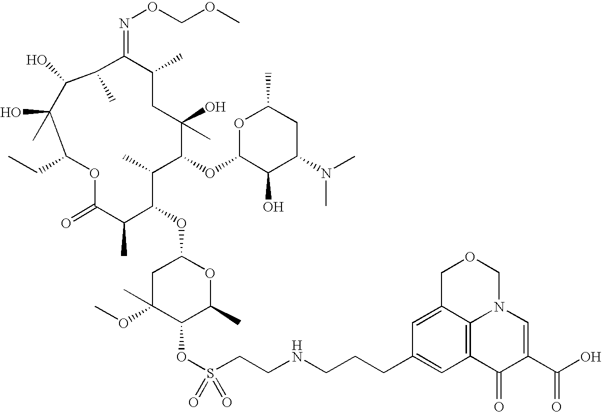

A stirred mixture of Intermediate 22 (0.09 g) and Intermediate 39 (0.044 g) in DMSO (1 mL) and triethylamine (0.042 mL) was heated at 50° C. for 2 h. The mixture was cooled, diluted with MeCN, and purified by mass directed automatic preparative HPLC to give, after freeze drying from dilute aqueous ammonia, the title compound as a white solid (0.061 g); ESMS m/z 1172.1 [M+H]⁺.

### Example 42

### 4″-O-{2-[3-(3-Carboxy-1,4-dihydro-1-methyl-4-oxo-6-quinolinyl) propylamino]ethanesulfonyl}-6-O-methylerythromycin A

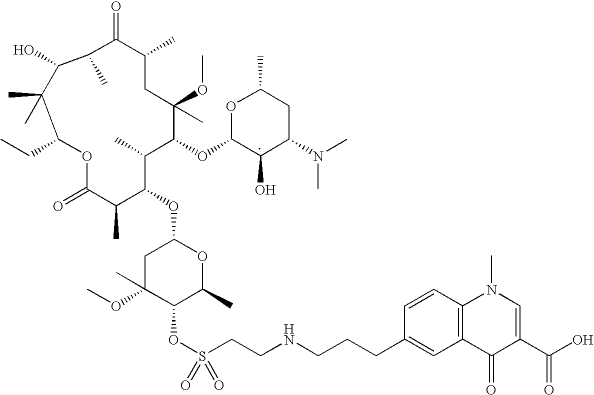

A stirred mixture of Intermediate 1 (0.126 g) and Intermediate 35 (0.084 g) in DMSO (0.75 mL) and triethylamine (0.062 mL) was heated at 50° C. for 40 min. The mixture was cooled, diluted with MeCN, and purified by mass directed automatic preparative HPLC to give, after freeze drying from dilute aqueous ammonia, the title compound as a white solid (0.08 g); ESMS m/z 1099.2 [M+H]⁺.

### Example 43

### 4″-O-{2-[3-(3-Carboxy-1,4-dihydro-1-methyl-4-oxo-6-quinolinyl) propylamino]ethanesulfonyl}-erythromycin A-(9E)-oxime-11,12-carbonate

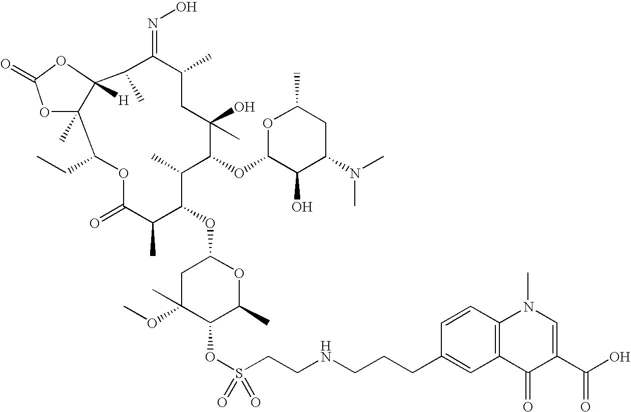

A stirred mixture of Intermediate 23 (0.13 g) and Intermediate 35 (0.084 g) in DMSO (0.75 mL) and triethylamine (0.062 mL) was heated at 50° C. for 2.5 h. The mixture was cooled, diluted with MeCN, and purified by mass directed automatic preparative HPLC to give, after freeze drying from dilute aqueous ammonia, the title compound as a white solid (0.056 g); ESMS m/z 1126.2 [M+H]⁺.

### Example 44

### 4″-O-{2-[3-(3-Carboxy-1,4-dihydro-1-ethenyl-4-oxo-6-quinolinyl) Propylamino]ethanesulfonyl}-6-O-methylerythromycin A

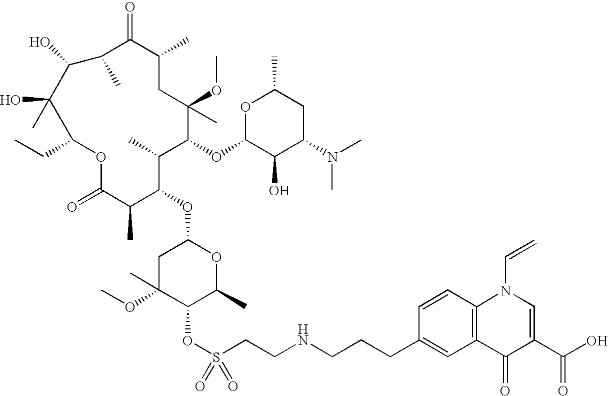

A stirred mixture of Intermediate 1 (0.126 g) and Intermediate 36 (0.087 g) in DMSO (0.75 mL) and triethylamine (0.062 mL) was heated at 50° C. for 2 h. The mixture was cooled, diluted with MeCN, and purified by mass directed automatic preparative HPLC to give, after freeze drying from dilute aqueous ammonia, the title compound as a white solid (0.122 g); ESMS m/z 1111.2 [M+H]⁺.

### Example 45

### 4″-O-{2-{[2-({2-[(3-Carboxy-7-chloro-1-cyclopropyl-4-oxo-1,4-dihydro-6-quinolinyl)amino]ethyl}oxy)ethyl]oxy}ethanesulfonyl}-6-O-methylerythromycin A

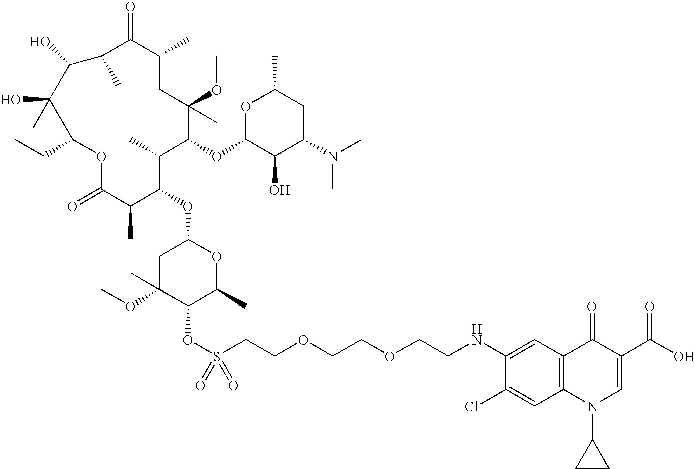

A stirred mixture of Intermediate 1 (0.084 g), Intermediate 37 (0.057 g) and potassium carbonate (0.048 g) in DMSO (0.5 mL) was heated at 60° C. After 17.5 h the mixture was cooled, diluted with MeCN, and purified by mass directed automatic preparative HPLC to give a product, which was taken up in THF (5 mL) and treated with an aqueous solution of lithium hydroxide (0.5 M) (0.30 mL). After 4.5 h ammonium chloride (0.088 g) was added and the mixture concentrated in vacuo to give a residue which was taken up in DCM containing several drops of MeOH. This mixture was filtered, concentrated in vacuo, and the resulting residue purified by mass directed automatic preparative HPLC, followed by flash chromatography (silica gel, 0-10% methanolic ammonia [2M] in DCM) to give, after freeze drying from dilute aqueous ammonia, the title compound as a cream solid (0.023 g); ESMS m/z 1205.0 [M+H]⁺.

### Example 46

### 4″-O-{2-{[2-({2-[(3-Carboxy-7-chloro-1-cyclopropyl-4-oxo-1,4-dihydro-6-quinolinyl)amino]ethyl}oxy)ethyl]oxy}ethanesulfonyl}-azithromycin

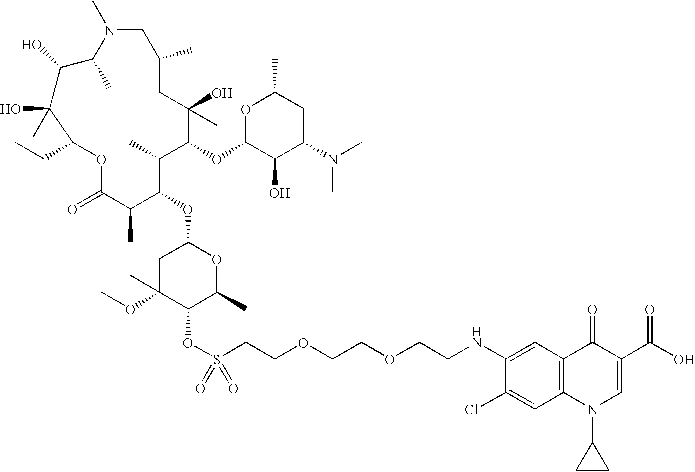

A stirred mixture of Intermediate 26 (0.352 g), Intermediate 37 (0.239 g) and potassium carbonate (0.203 g) in DMSO (2 mL) was heated at 60° C. Additional Intermediate 37 (0.08 g) was added after 15 min. After a further 17 h the mixture was heated to 70° C. Heating was continued for an additional 3 h, then the mixture was cooled, diluted with MeCN, and purified by mass directed automatic preparative HPLC to give a product, which was taken up in THF (20 mL) and treated with an aqueous solution of lithium hydroxide (0.5 M) (0.46 mL). Additional lithium hydroxide (0.5 M) (0.45 mL), and (1 mL) was added after 3 h and 5.5 h respectively. After a further 1.5 h ammonium chloride (0.06 g) was added and the mixture concentrated in vacuo to give a residue which was taken up in DCM containing several drops of MeOH. This mixture was filtered, concentrated in vacuo, and the resulting residue purified by mass directed automatic preparative HPLC, followed by flash chromatography (silica gel, 0-10% methanolic ammonia [2M] in DCM) to give, after freeze drying from dilute aqueous ammonia, the title compound as a cream solid (0.022 g); ESMS m/z 1206.0 [M+H]⁺.

### Example 47

### 4″-O-{2-(2-[(2-{[2-(3-Carboxy-1-ethyl-4-oxo-1,4-dihydro-6-quinolinyl)ethyl]oxy}ethyl)oxy]ethylamino)ethanesulfonyl}-erythromycin A-9E)-O-methoxymethyloxime

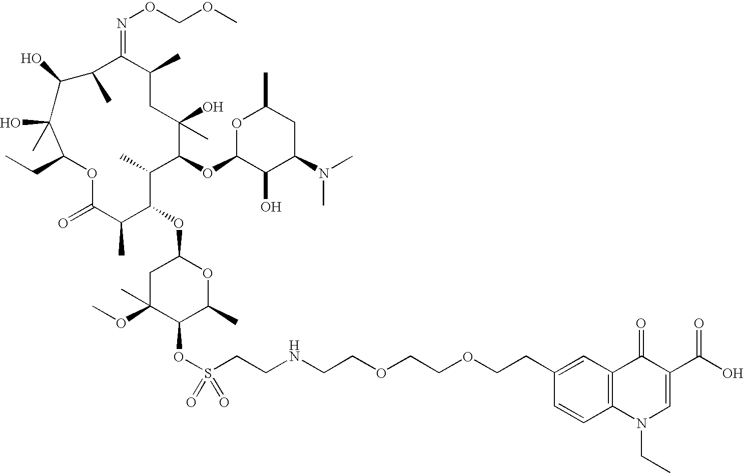

A stirred mixture of Intermediate 22 (0.066 g) and Intermediate 38 (0.043 g) in DMSO (0.75 mL) and triethylamine (0.031 mL) was heated at 50° C. Additional triethylamine (0.010 mL), (0.010 mL), (0.010 mL), and (0.031 mL) was added after 2.5 h, 4 h, 22 h, and 23 h respectively. Heating was continued for a further 50 min, then the mixture was cooled, diluted with MeCN, and purified by mass directed automatic preparative HPLC to give, after freeze drying from dilute aqueous ammonia, the title compound as a white solid (0.033 g); ESMS m/z 1232.1 [M+H]⁺.

### Example 48

### 4″-O-{2-(2-[(2-{[2-(3-Carboxy-1-ethyl-4-oxo-1,4-dihydro-4-quinolinyl)ethyl]oxy}ethyl)oxy]ethylamino)ethanesulfonyl}-6-O-methylerythromycin A

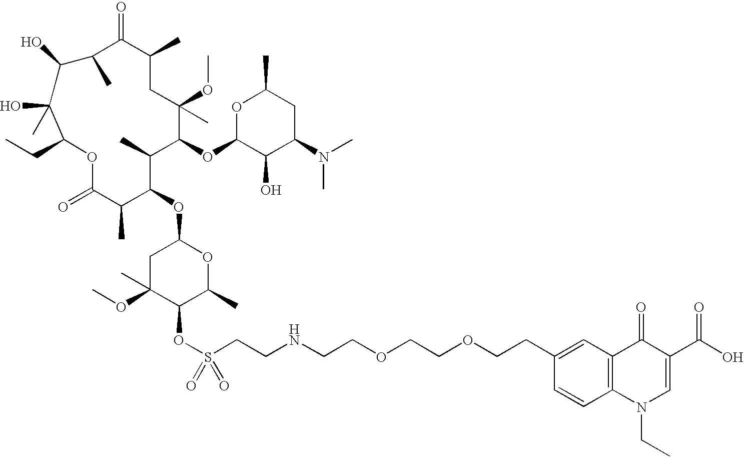

A stirred mixture of Intermediate 1 (0.063 g) and Intermediate 38 (0.043 g) in DMSO (0.75 mL) and triethylamine (0.031 mL) was heated at 50° C. for 4.5 h. The mixture was cooled, diluted with MeCN, and purified by mass directed automatic preparative HPLC to give, after freeze drying from dilute aqueous ammonia, the title compound as a white solid (0.067 g); ESMS m/z 1187.1 [M+H]⁺.

### Example 49

### 4″-O-{2-[3-(3-Carboxy-1,4-dihydro-1-methoxy-4-oxo-6-quinolinyl) propylamino]ethanesulfonyl}-6-O-methylerythromycin A

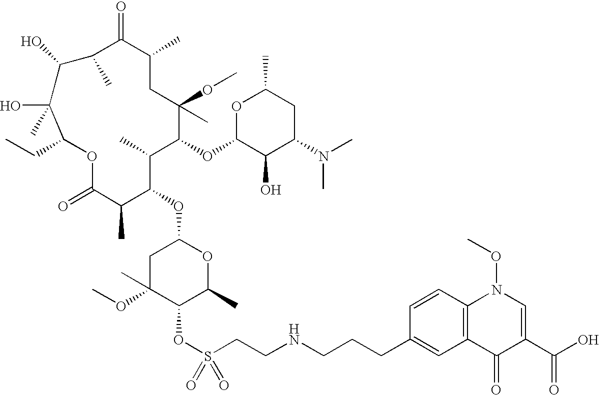

A stirred mixture of Intermediate 40 (0.047 g) and Intermediate 1 (0.084 g) in DMSO (0.5 mL) and triethylamine (0.04 mL) was heated at 60° C. After 6 h the mixture was cooled and purified by mass directed automatic preparative HPLC followed by chromatography (silica gel, 10 to 18% [9:1 MeOH/20 M aq. ammonia] in DCM to give the title compound as a white solid (0.054 g); ESMS m/z 1114.9 [M+H]⁺.

### Example 50

### 4″-O-{2-({3-[3-Carboxy-1-methoxy-4-oxo-1,4-dihydro-6-quinolinyl]propyl}amino)ethanesulfonyl}-erythromycin A-(9E)-O-methoxymethyloxime

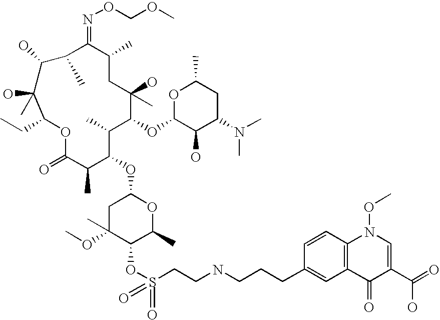

A stirred mixture of Intermediate 22 (0.15 g) and Intermediate 40 (0.08 g) in DMSO (0.75 mL) and triethylamine (0.07 mL) was heated at 60° C. After 20 hours the mixture was cooled, diluted with DCM, filtered and then flash chromatographed (silica gel, 10-16% (10% 20 M aq. ammonia in MeOH) in DCM). The impure product was purified by mass directed automatic preparative HPLC and then freeze drying from dilute aqueous ammonia to give the title compound as a white solid (0.03 g); ESMS m/z 1160.0 [M+H]⁺.

### Example 51

### 4″-O-{2-[2-(3-Carboxy-1,4-dihydro-1-ethyl-4-oxo-7-quinolinyloxy)ethylamino]ethanesulfonyl}-6-O-methylerythromycin A formate

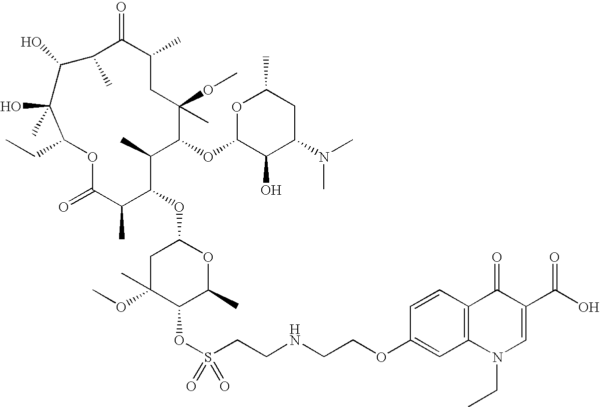

A stirred mixture of Intermediate 1 (0.168 g) and Intermediate 41 (0.105 g) in DMSO (0.5 mL), water (1 drop) and triethylamine (0.084 mL) was heated at 50° C. After 7 h the mixture was cooled, diluted with MeCN, and purified by mass directed automatic preparative HPLC to give, after freeze drying from dilute aqueous ammonia, the title compound as a white solid (0.147 g); ESMS m/z 1115.0 [M+H]⁺.

### Example 52

### 4″-O-{2-[2-(3-Carboxy-1,4-dihydro-1-ethyl-4-oxo-7-quinolinyloxy)ethylamino]ethanesulfonyl}erythromycin A-(9E)-oxime formate

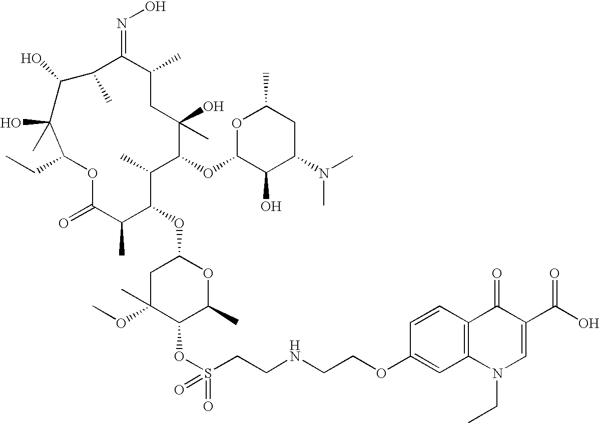

A stirred mixture of Intermediate 18 (0.168 g) and Intermediate 41 (0.105 g) in DMSO (0.5 mL), water (1 drop), and triethylamine (0.084 mL) was heated at 50° C. After 5 h the mixture was cooled, diluted with MeCN, and purified by mass directed automatic preparative HPLC to give, after freeze drying from dilute aqueous ammonia, the title compound as a white solid (0.114 g); ESMS m/z 1116.0 [M+H]⁺.

### Example 53

### 4″-O-{2-[2-(3-Carboxy-1,4-dihydro-1-ethyl-4-oxo-7-quinolinyloxy)ethylamino]ethanesulfonyl}erythromycin A-11,12-carbonate formate

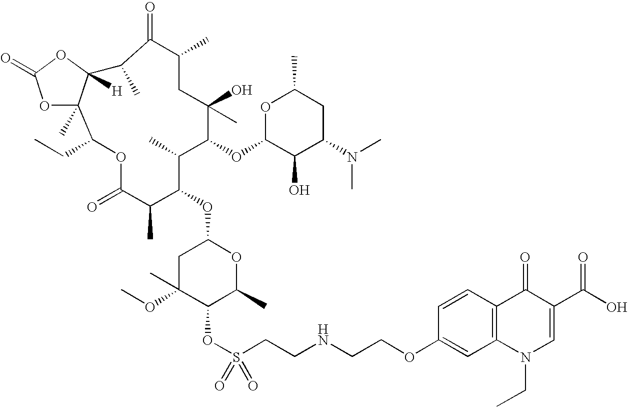

A stirred mixture of Intermediate 27 (0.17 g) and Intermediate 41 (0.105 g) in DMSO (0.5 mL), water (1 drop), and triethylamine (0.084 mL) was heated at 50° C. After 5 h the mixture was cooled, diluted with MeCN, and purified by mass directed automatic preparative HPLC to give, after freeze drying from dilute aqueous ammonia, the title compound as a white solid (0.066 g); ESMS m/z 1127.0 [M+H]⁺.

### Example 54

### 4″-O-{[2-({2-[(2-Carboxy-5-methyl-1-oxo-6,7-dihydro-1H,5H-pyrido[3,2,1-ij]quinolin-9-yl)amino]ethyl}amino)ethyl]sulfonyl}-6-O-methylerythromycin A

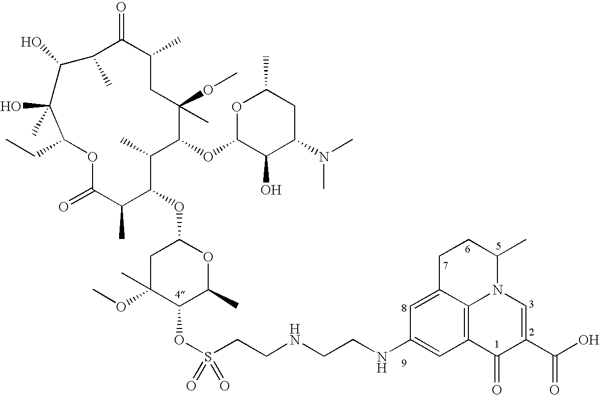

A stirred mixture of Intermediate 42 (0.085 g), Intermediate 1 (0.164 g) and triethylamine (0.246 mL) in DMSO (1 mL) was heated at 50° C. overnight. The reaction mixture was concentrated and purified by mass directed automatic preparative HPLC followed by chromatography (silica gel, 0 to 15% [9:1 MeOH/20 M aq. ammonia] in DCM to give the title compound as a pale yellow solid (0.12 g); ESMS m/z 1139.6 [M+H]⁺.

### Example 55

### 4″-O-{2-[{2-(3-Carboxy-1,4-dihydro-1-ethyl-6-fluoro-4-oxo-[1,8]naphthyridin-7-ylamino)ethyl}amino]ethanesulfonyl-erythromycin A-(9E)-oxime

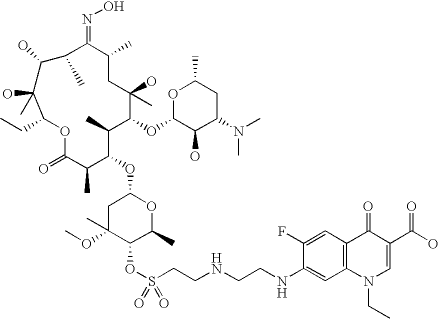

A stirred mixture of Intermediate 18 (0.1 g) and Intermediate 3 (0.071 g) in DMSO (1 mL) and triethylamine (0.035 mL) and water (1 drop) was heated at 60° C. After 3.5 hours the mixture was cooled, filtered, the insoluble extracted with MeCN. The combined soluble material was purified by mass directed automatic preparative HPLC to give, after freeze drying from dilute aqueous ammonia, the title compound as a white solid (0.044 g); ESMS m/z 1133.8 [M+H]⁺.

### Example 56

### 4″-O-{2-[{2-(3-Carboxy-1,4-dihydro-1-ethyl-4-oxo-[1,8]naphthyridin-7-ylamino)ethyl}amino]ethanesulfonyl}-}-6-O-methylerythromycin A

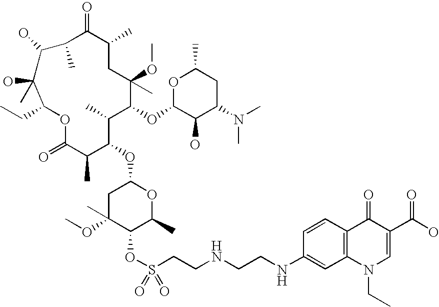

A stirred mixture of Intermediate 1 (0.1 g) and Intermediate 43 (0.07 g) in DMSO (1 mL) and triethylamine (0.033 mL) and water (1 drop) was heated at 50° C. After 19 hours the mixture was cooled and purified by mass directed automatic preparative HPLC to give, after freeze drying from dilute aqueous ammonia, the title compound as a white solid (0.043 g); ESMS m/z 1114.8 [M+H]⁺.

### Example 57

### 4″-O-{2-[3-(3-Carboxy-1,4-dihydro-1-ethyl-4-oxo-6-quinolinyl) propylamino]ethanesulfonyl}-erythromycin A (9E)-O-methyloxime

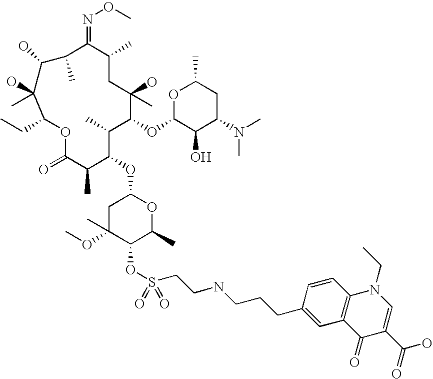

A stirred mixture of Intermediate 6 (0.068 g) and Intermediate 44 (0.1 g) in DMSO (0.5 mL) and triethylamine (0.05 mL) was heated at 50° C. After 4 h the mixture was cooled, diluted with DCM and filtered through celite. The filtrate was loaded onto a silica column and eluted with 10 to 18% [9:1 MeOH/20 M aq. ammonia] in DCM followed by mass directed automatic preparative HPLC to give, after freeze drying from dilute aqueous ammonia, the title compound as a white solid (0.069 g); ESMS m/z 1127.7 [M+H]⁺.

### Example 58

### 4″-O-{2-[2-(3-Carboxy-1-ethyl-6-fluoro-4-oxo-1,4-dihydro-[1,8]naphthyridin-7-ylamino)ethyl}amino]ethanesulfonyl}-erythromycin A (9E)-O-methyloxime

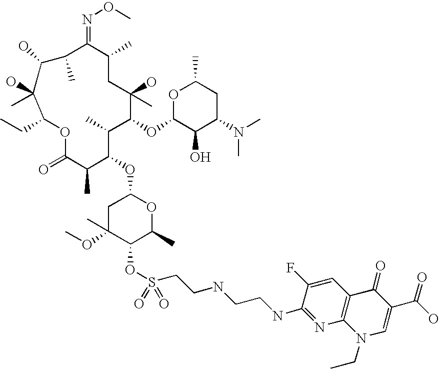

A stirred mixture of Intermediate 3 (0.071 g) and Intermediate 44 (0.1 g) in DMSO (0.5 mL) and triethylamine (0.05 mL) was heated at 50° C. After 24 h the mixture was cooled, diluted with DCM and filtered through celite. The filtrate was concentrated by evaporation and purified by mass directed automatic preparative HPLC to give, after freeze drying from dilute aqueous ammonia, the title compound as a white solid (0.072 g); ESMS m/z 1177.7 [M+H]⁺.

### Example 59

### 4″-O{2-{2-({3-(3-Carboxy-1-ethyl-4-oxo-1,4-dihydro-6-quinolinyl)]propyl}oxy)ethylamino}ethanesulfonyl}-azithromycin triformate

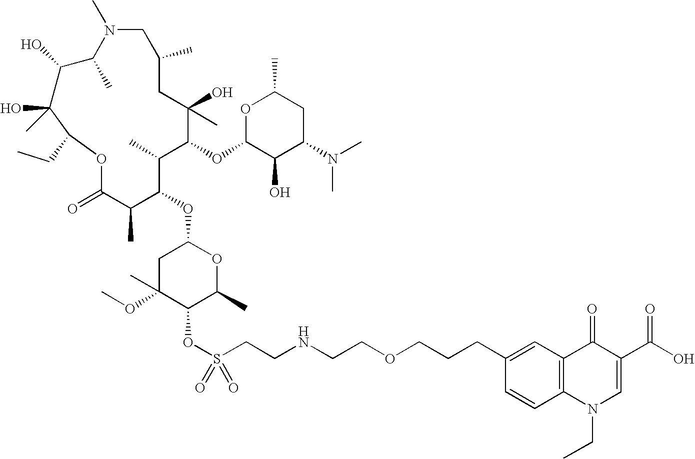

A stirred mixture of Intermediate 26 (0.084 g) and Intermediate 45 (0.087 g) in DMSO (0.5 mL) and triethylamine (0.056 mL) was heated at 50° C. for 4 h. The mixture was cooled, diluted with MeCN, and purified by mass directed automatic preparative HPLC to give, after freeze drying from dilute aqueous ammonia, the title compound as a white solid (0.05 g); ESMS m/z 1158.1 [M+H]⁺.

### Example 60

### 4′-O-{2-{[2-({3-[3-Carboxy-1-cyclopropyl-4-oxo-1,4-dihydro-6-quinolinyl]propyl}oxy)ethyl]oxy}ethanesulfonyl}-azithromycin

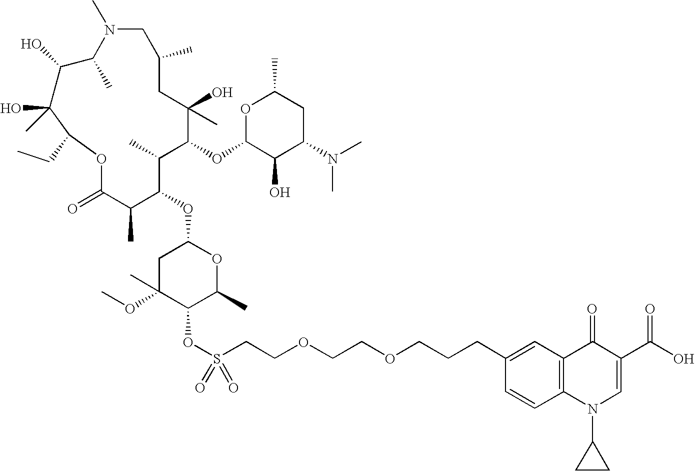

A stirred mixture of crude Intermediate 49 (0.674 g), Intermediate 47 (0.551 g) and potassium carbonate (0.01 g) in DMSO (1.5 mL) was heated at 60° C. After 21 h the mixture was cooled, diluted with EtOAc, and washed with water. The organic extracts were dried (Na₂SO₄), filtered, and concentrated in vacuo to give a residue which was purified by flash chromatography (silica gel, 0.5-8% methanolic ammonia [2M] in DCM), followed by mass directed automatic preparative HPLC to give a white solid (0.315 g); ESMS m/z 1281.2 [M+H]⁺. This material was taken up in DCM (5 mL) and treated successively with pyrrolidine (1.64 mL) and tetrakis(triphenylphosphine)palladium (0.028 g). The mixture was stirred under argon for 3.5 h, then additional tetrakis(triphenylphosphine)palladium (0.028 g) was added. Stirring was continued for a further 3.5 h then the mixture was concentrated in vacuo to give a residue which was taken up in water (20 mL) and EtOAc (20 mL). Aqueous orthophosphoric acid solution (1M) (3.3 mL) was added to take the mixture to pH 4, and the solution extracted with EtOAc (×3). To the aqueous solution was added trisodium orthophosphate (0.2 g) to take the solution to pH 8, and the aqueous then extracted with DCM (×3). The DCM extracts were combined, dried (Na₂SO₄), filtered, and concentrated in vacuo to give a residue which was purified by flash chromatography (silica gel, 0-18% [9:1 MeOH/20M aqueous ammonia] in DCM) to give a white solid (0.242 g); ESMS m/z 579.1 [M+2H]⁺⁺. This material was taken up in chloroform (10 mL) and treated with formic acid (0.032 mL) and formaldehyde (37% aq) (0.035 mL). The mixture was stirred with heating to reflux for 2 h, then diluted with MeCN and water, and concentrated in vacuo to give a residue which was purified by flash chromatography (silica gel, 0-18% [9:1 MeOH/20M aqueous ammonia] in DCM) to give, after freeze drying, the title compound as a white solid (0.204 g); ESMS m/z 586.3 [M+2H]⁺⁺.

### Example 61

### 4″-O-{2-{[2-({3-[3-Carboxy-1-ethyl-4-oxo-1,4-dihydro-6-quinolinyl]propyl}oxy)ethyl]oxy}ethanesulfonyl}-azithromycin

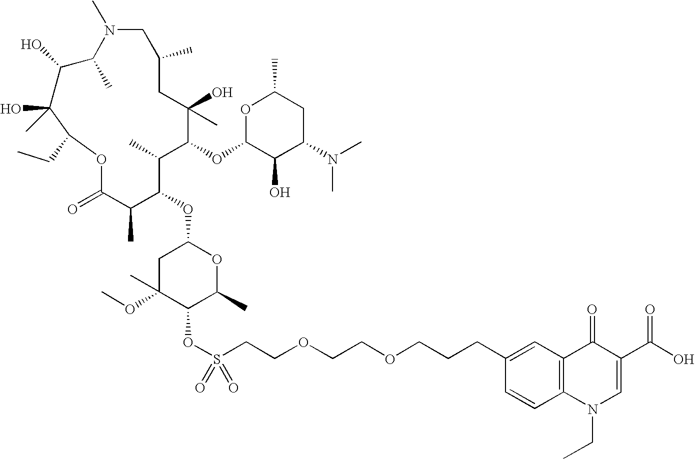

A stirred mixture of crude Intermediate 50 (0.747 g), Intermediate 46 (0.541 g) and potassium carbonate (0.01 g) in DMSO (1 mL) was heated at 60° C. After 13.5 h dimethylsulfoxide (0.5 mL) was added, the mixture sonicated, and heating continued. After a further 36 h the mixture was cooled, diluted with EtOAc, and washed with water. The organic extracts were dried (Na₂SO₄), filtered, and concentrated in vacuo to give a residue which was purified by flash chromatography (silica gel, 0-100% THF in diethyl ether), followed by mass directed automatic preparative HPLC to give a white solid (0.206 g); ESMS m/z 1353.1 [M+H]⁺. This material was taken up in DCM (3 mL) and treated successively with pyrrolidine (1 mL) and tetrakis(triphenylphosphine)palladium (0.017 g). The mixture was stirred under argon for 4 h, then additional tetrakis(triphenylphosphine)palladium (0.02 g) was added. Stirring was continued for a further 4 h then the mixture was concentrated in vacuo to give a residue which was taken up in water (20 mL) and EtOAc (20 mL). Aqueous orthophosphoric acid solution (1M) (2 mL) was added to take the mixture to pH 4, and the solution washed with EtOAc (×3). To the aqueous solution was added trisodium orthophosphate (0.13 g) to take the solution to pH 8, and the aqueous then extracted with DCM (×3). The DCM extracts were combined, dried (Na₂SO₄), filtered, and concentrated in vacuo to give a residue which was purified by flash chromatography (silica gel, 0-18% [9:1 MeOH/20 M aq. ammonia] in DCM) to give a white solid (0.158 g); ESMS m/z 573.1 [M+2H]⁺. This material was taken up in chloroform (10 mL) and treated with formic acid (0.021 mL) and formaldehyde (37% aq) (0.023 mL). The mixture was stirred with heating to reflux for 2 h, then diluted with MeCN and water, and concentrated in vacuo to give a residue which was purified by flash chromatography (silica gel, 0-18% [9:1 MeOH/20 M aq. ammonia] in DCM) to give, after freeze drying from dilute aqueous ammonia, the title compound as a white solid (0.143 g); ESMS m/z 580.3 [M+2H]⁺⁺.

### Example 62

### 4″-O-{2-{[2-({2-[(3-Carboxy-1-cyclopropyl-4-oxo-1,4-dihydro-6-quinolinyl)amino]ethyl}oxy)ethyl]oxy}ethanesulfonyl}-azithromycin

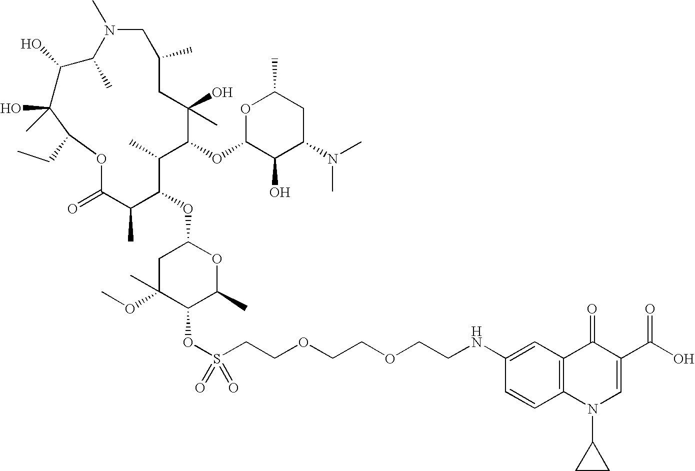

and

### Example 63

### 4″-O-{2-{[2-({2-[(3-Carboxy-1-cyclopropyl-4-oxo-1,4-dihydro-6-quinolinyl)methylamino]ethyl}oxy)ethyl]oxy}ethanesulfonyl}-azithromycin

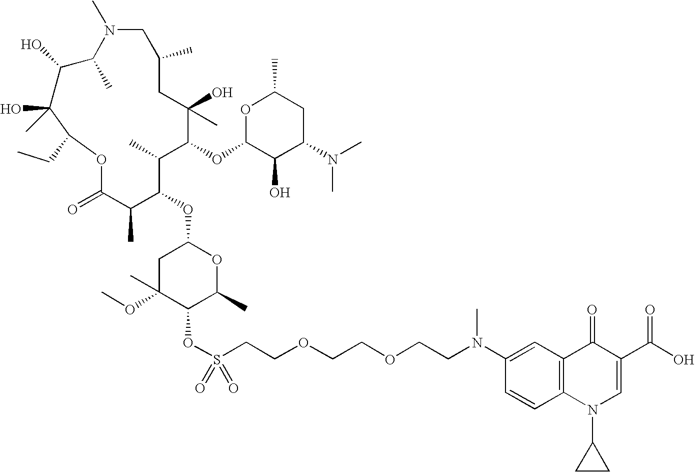

A stirred mixture of crude Intermediate 51 (0.5 g), Intermediate 48 (0.466 g) and potassium carbonate (0.007 g) in DMSO (2 mL) was heated at 60° C. After 14 h the mixture was cooled, diluted with EtOAc, and washed with water. The organic extracts were dried (Na₂SO₄), filtered, and concentrated in vacuo to give a residue which was purified by flash chromatography (silica gel, 0-20% MeOH in DCM) to give a crude pale yellow solid (0.298 g) which was used without further purification; ESMS m/z 1415.9 [M+H]⁺. A portion of this material (0.269 g) in MeOH (20 mL) was hydrogenated over 10% palladium on charcoal (0.06 g) for 23 h. Additional 10% palladium on charcoal (0.03 g) was added and hydrogenation continued. After a further 28 h the mixture was filtered through a Celite pad, which was then washed with DCM and MeOH. The combined filtrates were concentrated in vacuo to give a residue which was purified by flash chromatography (silica gel, 0-16% [9:1 MeOH/20M aqueous ammonia] in DCM) to give a crude pale yellow solid (0.175 g); ESMS m/z 579.7 [M+2H]⁺⁺. This material was taken up in chloroform (6 mL) and treated with formic acid (0.023 mL) and formaldehyde (37% aq.) (0.017 mL). The mixture was stirred with heating to reflux for 21 h, then diluted with MeCN and water and concentrated in vacuo to give a residue which was purified by mass directed automatic preparative HPLC to give two crude products. Each was separately further purified by flash chromatography (silica gel, 0-16% [9:1 MeOH/20M aqueous ammonia] in DCM) to give, after freeze drying from dilute aqueous ammonia, the mono-methylated product Example 62 as a cream solid (0.052 g); ESMS m/z 586.7 [M+2H]⁺⁺ and the di-methylated product Example 63 as a cream solid (0.006 g); ESMS m/z 1185.7 [M+H]⁺.

### Example 64

### 4″-O-{2-[2-(3-Carboxy-1,4-dihydro-1-ethyl-6-fluoro-4-oxo-[1,8]naphthyridin-7-ylamino)ethylamino]ethanesulfonyl}-erythromycin A-(9E)-O-cyanomethyloxime

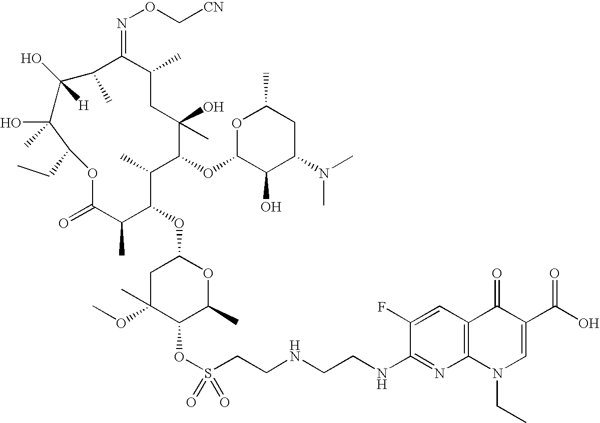

A stirred mixture of Intermediate 52 (0.25 g) and Intermediate 3 (0.118 g) in DMSO (10 mL) and triethylamine (0.5 mL) was heated at 50° C. and stirred overnight, cooled and then concentrated. The residue was purified by mass directed automatic preparative HPLC and then flash chromatography (silica gel, 3.75-15% [10% 0.880 aqueous ammonia in MeOH] in DCM) to give the title compound as a white solid (0.135 g); ESMS m/z 1172.8 [M+H]⁺.

### Example 65

### 4′-O-{2-[3-(3-Carboxy-1,4-dihydro-1-dimethylamino-4-oxo-6-quinolinyl)propylamino]ethanesulfonyl}-erythromycin A-(9E)-O-(5-methylisoxazol-3-yl)-methyloxime

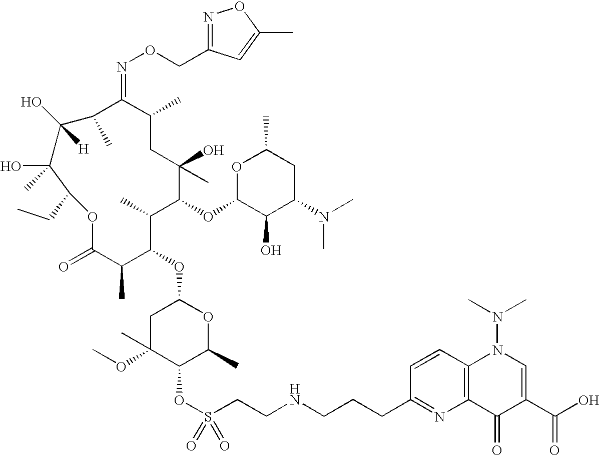

Using a similar procedure to that described in Example 64, Intermediate 53 (0.26 g), and Intermediate 4 (0.13 g) gave the title compound as a yellow solid (0.209 g); ESMS m/z 1223.8 [M+H]⁺.

### Example 66

### 4″-O-{2-[3-(3-Carboxy-1,4-dihydro-1 ethyl-4-oxo-6-quinolinyl)propylamino]ethanesulfonyl}-erythromycin A-(9E)-O—(N,N-dimethylacetamido)-oxime

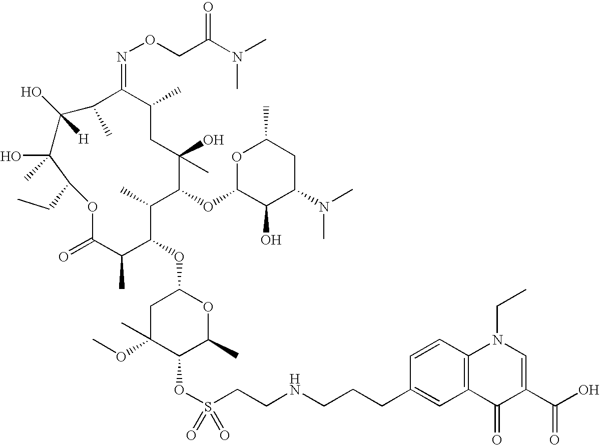

Using a similar procedure to that described in Example 64, Intermediate 54 (0.304 g) and Intermediate 6 (0.143 g) gave the title compound as a white solid (0.167 g); ESMS m/z 1198.9 [M+H]⁺.

### Example 67

### 4″-O-{2-({3-[3-carboxy-1-ethyl-4-oxo-1,4-dihydro-6-quinolinyl]propyl}amino)ethanesulfonyl}-erythromycin A (9E)-O-2-(diethylamino)ethyloxime

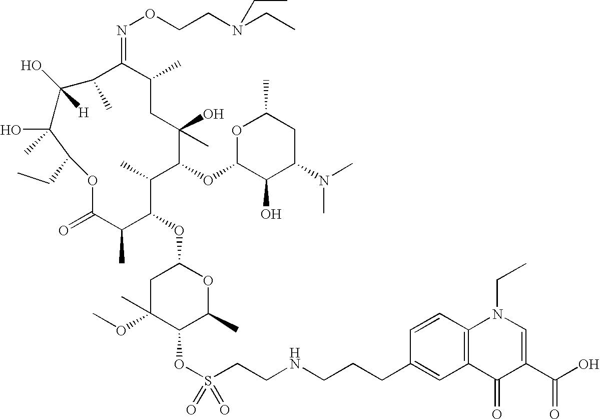

A mixture of Intermediate 55 (0.2 g), Intermediate 6 (0.116 g) and triethylamine (0.4 mL) in DMSO (3 mL) were stirred together at 50° C. for 16 hours. The mixture was evaporated to dryness and the residue was purified by mass directed automatic preparative HPLC followed by chromatography on silica gel eluting with 0-12% (9:1 MeOH/20M aqueous ammonia) in DCM to give the title compound as a white solid (0.144 g); ESMS m/z 607.2 [M+2H]²⁺.

### Example 68

### 4″-O-{2-{[2-(3-Carboxy-1,4-dihydro-1-ethyl-6-fluoro-4-oxo-[1,8]naphthyridin-7-ylamino)ethyl}amino]ethanesulfonyl}-erythromycin A (9E)-O-2-(diethylamino)ethyloxime

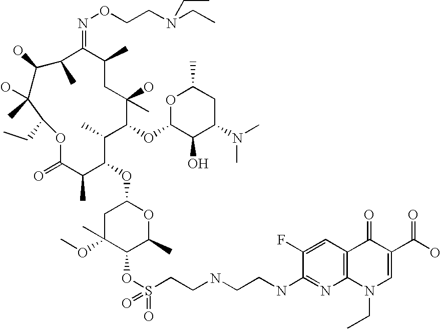

Using a similar procedure to that described in Example 67, Intermediate 55 (0.2 g) and Intermediate 3 (0.122 g) were reacted together for 4 days to give title compound as a white solid (0.104 g); ESMS m/z 617.3 [M+2H]²⁺.

### Example 69

### 4″-O-{2-[3-(3-Carboxy-1,4-dihydro-1-Dimethylamino-4-oxo-6-quinolinyl)propylamino]ethanesulfonyl}-erythromycin A (9E)-2-(diethylamino)ethyloxime

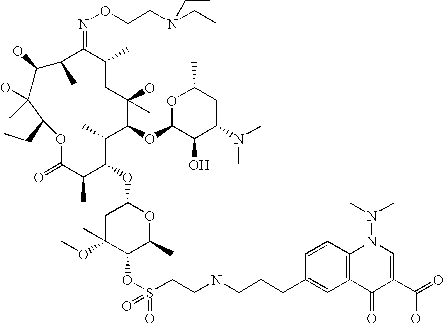

Using a similar procedure to that described in Example 67, Intermediate 55 (0.2 g) and Intermediate 4 (0.102 g) gave the title compound as a white solid (0.081 g); ESMS m/z 614.7 [M+2H]²⁺.

### Example 70

### 4″-O-[(2-{[3-(6-Carboxy-7-oxo-1H,7H-[1,3]oxazino[5,4,3-ij]quinolin-9-yl)propyl]amino}ethanesulfonyl]-erythromycin A (9E)-oxime

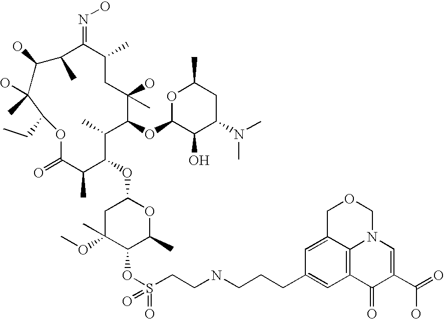

Using a similar procedure to that described in Example 67, but with a 6 h reaction time, Intermediate 18 (0.2 g) and Intermediate 39 (0.13 g) gave the title compound as a white solid (0.091 g); ESMS m/z 564.4 [M+2H]²⁺.

### Example 71

### 4″-O-[(2-{[3-(6-carboxy-7-oxo-1H,7H-[1,3]oxazino[5,4,3-ij]quinolin-9-yl)propyl]amino}ethanesulfonyl]-erythromycin A (9E)-O-cyanomethyloxime

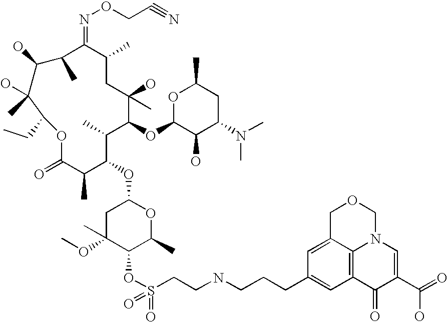

Using a similar procedure to that described in Example 67, but with a 6 h reaction time, Intermediate 52 (0.209 g) and Intermediate 39 (0.13 g) gave the title compound as a white solid (0.137 g); ESMS m/z 583.9 [M+2H]²⁺.

### Example 72

### 4″-O-{2-[3-(7-Carboxy-8-oxo-3,4-dihydro-1H,8H-[1,4]oxazepino[6,5,4-ij]quinoline-10-yl)propylamino]ethanesulfonyl}-6-O-methyl-erythromycin A

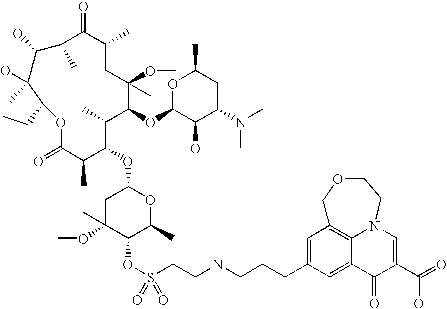

A stirred mixture of Intermediate 1 (0.167 g) and Intermediate 28 (0.105 g) in DMSO (0.8 mL) and triethylamine (0.1 mL) was heated at 50° C. After 3 h the mixture was cooled and concentrated to low volume by evaporation under reduced pressure and freeze drying. The residue was purified by chromatography (silica gel, 0-15% methanolic ammonia [2M] in DCM) to give the title compound as a white solid (0.175 g); ESMS m/z 1140.8 [M+H]⁺.

### Example 73

### 4″-O-{2-({3-[3-Carboxy-1-ethyl-4-oxo-1,4-dihydro-6-quinolinyl]propyl}methylamino)ethanesulfonyl}-erythromycin A-(9E)-oxime formats salt

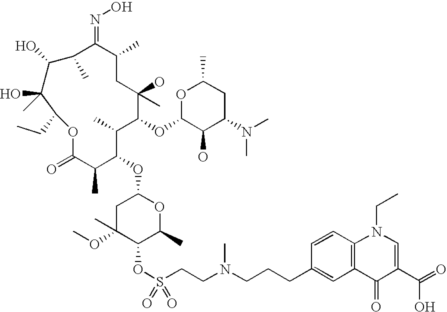

Example 19 (0.05 g) and formaldehyde (37% in water, 0.007 mL) in DCM (3 mL) was hydrogenated at atmospheric pressure over 10% palladium on carbon (0.04 g) for 17 h. The catalyst was filtered off and the solution combined with further Example 19 (0.324 g) and formaldehyde (37% in water, 0.065 mL) in DCM (10 mL) and stirred at 20 C for 1 h. The solution was hydrogenated at atmospheric pressure over 10% palladium on carbon (0.3 g) for 17 h. The catalyst was filtered off and the solution concentrated in vacuo to give a residue which was purified by mass directed automatic preparative HPLC to give the title compound as a white solid, NMR showing 3 equivalents of formic acid, (0.13 g); ESMS m/z 1127.8 [M+H]⁺.

### Biological Data

Using a standard broth dilution method in microtitre, compounds were tested for antibacterial activity. The compounds in the above examples gave minimum inhibitory concentrations (MICs) less than 1 microgram per millilitre against erythromycin-sensitive and erythromycin-resistant strains of Streptococcus pneumoniae and Streptococcus pyogenes.

However, it will appreciated by person skilled in the art that compounds of the invention may have different levels of activity against different strains of the same bacteria.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims:

1. A compound of formula (I) 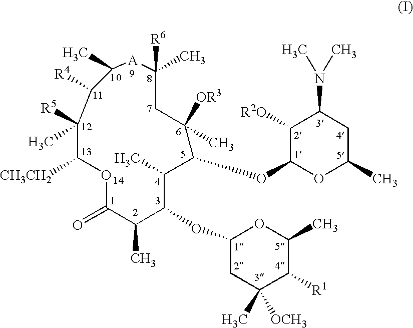
wherein
A is a bivalent radical —C(O)—, —N(R⁷)—CH₂—, —CH(NR⁸R⁹)— or —C(═NR¹⁰)—, or A and R⁴ taken together with the intervening atoms form a cyclic group having the following formula: 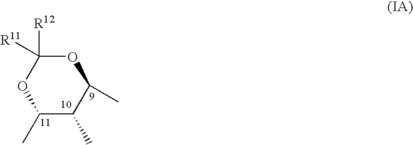
R¹ is —OS(O)₂(CH₂)₂U¹R¹³, 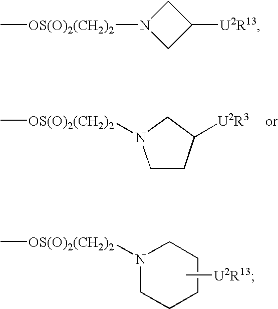
R² is hydrogen or a hydroxyl protecting group;
R³ is hydrogen, C₁₋₄alkyl, or C₃₋₆alkenyl optionally substituted by 9- or 10-membered fused bicyclic heteroaryl;
R⁴ is hydroxy, C₃₋₆alkenyloxy optionally substituted by 9- or 10-membered fused bicyclic heteroaryl, or C₁₋₆alkoxy optionally substituted by C₁₋₆alkoxy or —O(CH₂)_{d}NR⁷R¹⁴, or R⁴ and A taken together with the intervening atoms form a cyclic group of formula (IA),
R⁵ is hydroxy, or
R⁴ and R⁵ taken together with the intervening atoms form a cyclic group having the following formula: 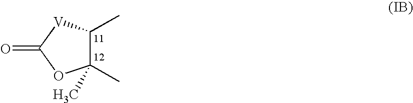
wherein V is a bivalent radical —CH₂—, —CH(CN)—, —O—, —N(R¹⁵)— or —CH(SR¹⁵)—;
R⁶ is hydrogen or fluorine;
R⁷ is hydrogen or C₁₋₆alkyl;
R⁸ and R⁹ are each independently hydrogen, C₁₋₆alkyl or —C(O)R¹⁶, or
R⁸ and R⁹ together form ═CH(CR¹⁶R¹⁷)ₑaryl, ═CH(CR¹⁶R¹⁷)ₑheterocyclyl, ═CR¹⁶R¹⁷ or ═C(R¹⁶)C(O)OR¹⁶, wherein the alkyl, aryl and heterocyclyl groups are optionally substituted by up to three groups independently selected from R¹⁸;
R¹⁰ is —OR¹⁹;
R¹¹ and R¹² are each independently hydrogen, C₁₋₆alkyl, heteroaryl, or aryl optionally substituted by one or two groups independently selected from hydroxyl and C₁₋₆alkoxy;
R¹³ is a heterocyclic group having one of the following formulae: 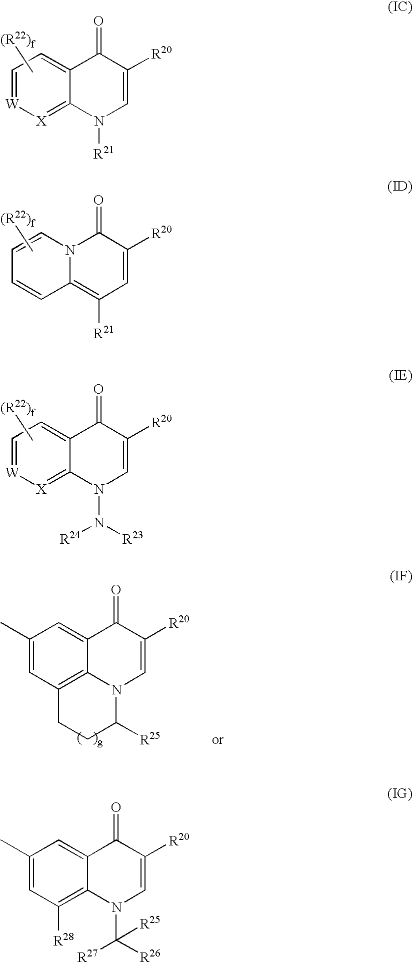
R¹⁴, R¹⁶ and R¹⁷ are each independently hydrogen or C₁₋₆alkyl;
R¹⁵ is hydrogen or C₁₋₄alkyl optionally substituted by a group selected from optionally substituted phenyl, optionally substituted 5- or 6-membered heteroaryl and optionally substituted 9- or I O-membered fused bicyclic heteroaryl;
R¹⁸ is halogen, cyano, nitro, trifluoromethyl, azido, —C(O)R²⁹, —C(O)OR²⁹, —OC(O)R²⁹, —OC(O)OR²⁹—NR³⁰C(O)R³¹, —C(O)NR³⁰R³¹, —NR³⁰R³¹, hydroxy, C₁₋₆alkyl, —S(O)ₕC₁₋₆alkyl, C₁₋₆alkoxy, —(CH₂)ᵢaryl or —(CH₂)ᵢheteroaryl, wherein the alkoxy group is optionally substituted by up to three groups independently selected from —NR¹⁶R¹⁷, halogen and —OR¹⁶, and the aryl and heteroaryl groups are optionally substituted by up to five groups independently selected from halogen, cyano, nitro, trifluoromethyl, azido, —C(O)R³², —C(O)OR³², —OC(O)OR³², —NR³³C(O)R³⁴, —C(O)NR³³R³⁴, —NR³³R³⁴, hydroxy, C₁₋₆alkyl and C₁₋₆alkoxy;
R¹⁹ is hydrogen, C₁₋₆alkyl, C₃₋₇cycloalkyl, C₃₋₆alkenyl, or a 5- or 6-membered heterocyclic group, wherein the alkyl, cycloalkyl, alkenyl, and heterocyclic groups are optionally substituted by up to three groups independently selected from optionally substituted 5- or 6-membered heterocyclic group, optionally substituted 5- or 6-membered heteroaryl, —OR³⁵, —S(O)ⱼR³⁵, —NR³⁵R³⁶, —CONR³⁵R³⁶, halogen and cyano;
R²⁰ is —C(O)OR³⁷, —C(O)NHR³⁷, —C(O)CH₂NO₂ or —C(O)CH₂SO₂R⁷;
R²¹ is hydrogen,
C₁₋₄alkyl optionally substituted by up to three groups independently selected from hydroxyl, C₁₋₄alkoxy and halogen,
C₁₋₄alkoxy,
C₂₋₆alkenyl,
C₃₋₇cycloalkyl, or
optionally substituted phenyl or benzyl, or
R²¹ and R⁴⁴ are linked to form the bivalent radical —(CH₂)ₖ—;
R²² is halogen, C₁₋₄alkyl, C₁₋₄thioalkyl, C₁₋₄alkoxy, —NH₂, —NH(C₁₋₄alkyl) or —N(C₁₋₄alkyl)₂;
R²³ and R²⁴ are each independently hydrogen, C₁₋₄alkyl or C₃₋₇cycloalkyl, wherein the alkyl and cycloalkyl groups are optionally substituted by up to three groups independently selected from hydroxy, cyano, C₁₋₄alkoxy, —CONR³⁸R³⁹ and —NR³⁸R³⁹, R²³ and R²⁴, together with the nitrogen atom to which they are bound, form a 5- or 6-membered heterocyclic ring optionally containing one additional heteroatom selected from oxygen, sulfur and N—R⁴⁰, or
R²³ is C₁₋₄alkyl, X is —C(R⁴⁴)—, and R²⁴ and R⁴⁴ are linked to form a cyclic group having the following formula: 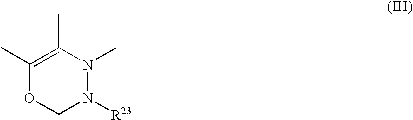
R²⁵ and R²⁶ are each independently hydrogen or methyl;
R²⁷ and R²⁸ are linked to form a bivalent radical —OCH₂—, —CH₂O—, —O(CH₂)₂—, —CH₂OCH₂— or —(CH₂)₂O—;
R²⁹ is hydrogen, C₁₋₁₀alkyl, —(CH₂)ₘaryl or —(CH₂)ₘheteroaryl;
R³⁰ and R³¹ are each independently hydrogen, —OR¹⁶, C₁₋₆alkyl, —(CH₂)ₙaryl or —(CH₂)ₙheterocyclyl;
R³² is hydrogen, C₁₋₁₀alkyl, —(CH₂)ₚaryl or —(CH₂)ₚheteroaryl;
R³³ and R³⁴ are each independently hydrogen, —OR¹⁶, C₁₋₆alkyl, —(CH₂)_{q}aryl or —(CH₂)_{q}heterocyclyl;
R³⁵ and R³⁶ are each independently hydrogen, C₁₋₄alkyl or C₁₋₄alkoxyC₁₋₄alkyl;
R³⁷ is hydrogen,
C₁₋₆alkyl optionally substituted by up to three groups independently selected from halogen, cyano, C₁₋₄alkoxy optionally substituted by phenyl or C₁₋₄alkoxy, —C(O)C₁₋₆alkyl, —C(O)OC₁₋₆alkyl, —OC(O)C₁₋₆alkyl, —OC(O)OC₁₋₆alkyl, —C(O)NR⁴¹R⁴², —NR⁴¹R⁴² and phenyl optionally substituted by nitro or —C(O)OC₁₋₆alkyl,
—(CH₂)ᵣC₃₋₇cycloalkyl,
—(CH₂)ᵣheterocyclyl,
—(CH₂)ᵣheteroaryl,
—(CH₂)ᵣaryl,
C₃₋₆alkenyl, or
C₃₋₆alkynyl;
R³⁸ and R³⁹ are each independently hydrogen or C₁₋₄alkyl;
R⁴⁰ is hydrogen or methyl;
R⁴¹ and R⁴² are each independently hydrogen or C₁₋₆alkyl optionally substituted by phenyl or —C(O)OC₁₋₆alkyl, or
R⁴¹ and R⁴², together with the nitrogen atom to which they are bound, form a 5- or 6-membered heterocyclic group optionally containing one additional heteroatom selected from oxygen, sulfur and N—R⁴⁰;
R⁴³ is hydrogen, C₁₋₄alkyl, C₃₋₇cycloalkyl, optionally substituted phenyl or benzyl, acetyl or benzoyl;
R⁴⁴ is hydrogen or R²², or, when X is —C(R⁴⁴)—, R⁴⁴ and R²⁴ may be linked to form a cyclic group of formula (IH) or R⁴⁴ and R²¹ may be linked to form the bivalent radical —(CH₂)ₖ—;
U¹ is a bivalent radical —Y(CH₂)ₛB—, —Y(CH₂)ₛ—, —Y(CH₂)ₛB(CH₂)ₜD-, —Y(CH₂)₅B(CH₂)ₜ—, —Y(CH₂)ₛB(CH₂)ₜD(CH₂)ᵤE- or —Y(CH₂)ₛB(CH₂)ₜD(CH₂)ᵤ—;
U² is U¹ or a bivalent radical —O—, —N(R⁴³)—, —S(O)ᵥ— or —CH₂—;
Y, B, D and E are each independently a bivalent radical —N(R⁴³)—, —O—, —S(O)ᵥ—, —N(R⁴³)C(O)—, —C(O)N(R⁴³)— or —N[C(O)R⁴³]—;
W and X are each independently —C(R⁴⁴)— or a nitrogen, with the proviso that W and X are not both nitrogen;
d is an integer from 2 to 4;
e, i, m, n, p, q and r are each independently integers from 0 to 4;
f, h, j and v are each independently integers from 0 to 2;
g is 0 or 1;
s, t and u are each independently integers from 2 to 5;
k is 2 or 3;
or a pharmaceutically acceptable derivative thereof.

2. A compound according to claim 1 wherein A is —C(O)—, —N(R⁷)—CH₂— or —C(═NR¹⁰)—, or A and R⁴ taken together with the intervening atoms form a cyclic group having the following formula:

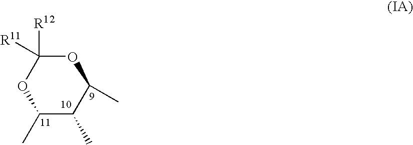

3. A compound according to claim 1 wherein R¹ is —OS(O)₂(CH₂)₂U¹R¹³ or

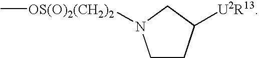

4. A compound according to claim 1 wherein U¹ is —Y(CH₂)ₛB—, —Y(CH₂)ₛ—, —Y(CH₂)ₛB(CH₂)ₜD- or —Y(CH₂)ₛB(CH₂)ₜD(CH₂)ᵤ—.

5. A compound according to claim 1 wherein s is 2 or 3.

6. A compound according to claim 1 wherein R¹³ is a heterocyclic group having one of the following formulae: 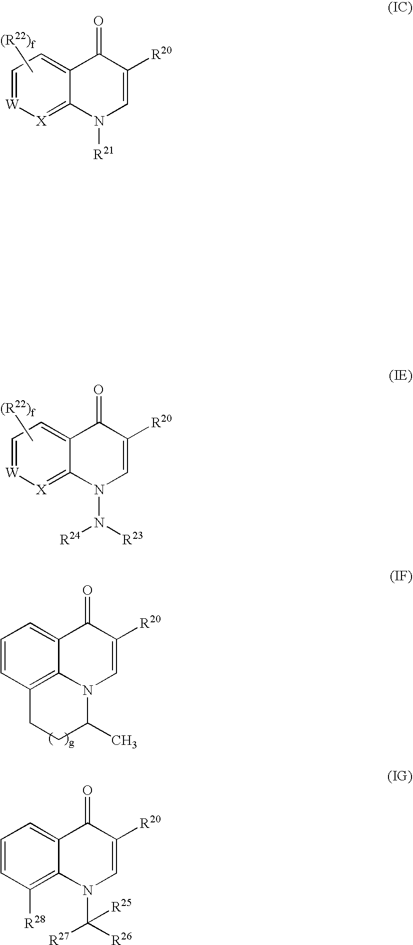
wherein R²¹ to R²⁸ are as defined in claim 1.

7. A compound according to claim 1 as defined in any one of Examples 1 to 54, or a pharmaceutically acceptable derivative thereof.

8. A compound selected from:
4″-O-{2-[3-(3-Carboxy-1,4-dihydro-1-ethyl-4-oxo-6-quinolinyl) propylamino]ethanesulfonyl}-6-O-methylerythromycin A,
4″-O-{2-[3-(3-Carboxy-1,4-dihydro-1-dimethylamino-4-oxo-6-quinolinyl)propylamino]ethanesulfonyl}-9-(S)-dihydroerythromycin A-9,11-ethylidene acetal,
4″-O-{2-[{2-(3-Carboxy-1,4-dihydro-1-ethyl-6-fluoro-4-oxo-[1,8]naphthyridin-7-ylamino)ethyl}amino]ethanesulfonyl}-6-O-methyl-erythromycin A formate,
4″-O-{2-[2-(3-Carboxy-1,4-dihydro-1-ethyl-4-oxo-[1,7]naphthyridin-6-ylsulfanyl)-ethylamino]ethanesulfonyl}-6-O-methyl-erythromycin A,
4″-O-{2-[3-(3-Carboxy-1,4-dihydro-1-dimethylamino-4-oxo-6-quinolinyl)propylamino]ethanesulfonyl}-6-O-methyl-erythromycin A,
4″-O-{2-[3-(3-Carboxy-1,4-dihydro-1-ethyl-4-oxo-[1,8]naphthyridin-6-yl)propylamino]ethanesulfonyl}-6-O-methyl-erythromycin A,
O-{2-[2-(3-Carboxy-1,4-dihydro-1-Dimethylamino-4-oxo-[1,7]naphthyridin-6-ylsulfanyl)-ethylamino]ethanesulfonyl}-6-O-methyl-erythromycin A formate,
4″-O-{2-[3-(6-Carboxy-7-oxo-2,3-dihydro-1H,7H-pyrido[3,2,1-ij]quinolin-9-yl)propylamino]ethanesulfonyl}-6-O-methylerythromycin A formate,
4″-O-{[(2-{[3-(6-Carboxy-3-methyl-7-oxo-1H,7H-[1,3]oxazino[5,4,3-ij]quinolin-9-yl)propyl]amino}ethyl)sulfonyl]-6-O-methylerythromycin A formate,
4″-O-{2-[3-(3-Carboxy-1,4-dihydro-1-(2,2,2-trifluoroethyl)-4-oxo-6-quinolinyl)propylamino]ethanesulfonyl}-6-O-methylerythromycin A,
4″-O-{2-[2-(3-Carboxy-1,4-dihydro-1-dimethylamino-4-oxo-6-quinolinyl)sulfanylethylamino]ethanesulfonyl}-6-O-methylerythromycin A,
4″-O-[(2-{[3-(7-Carboxy-8-oxo-3,4-dihydro-2H,8H-[1,4]oxazepino[2,3,4-ij]quinolin-10-yl)propyl]amino}ethyl)sulfonyl]-6-O-methylerythromycin A,
4″-O-[(2-{[3-(6-Carboxy-3,3-dimethyl-7-oxo-1H,7H-[1,3]oxazino[5,4,3-ij]quinolin-9-yl)propyl]amino}ethyl)sulfonyl]-6-O-methylerythromycin A,
4″-O-{[2-({2-[(2-Carboxy-5-methyl-1-oxo-6,7-dihydro-1H,5H-pyrido[3,2,1-ij]quinolin-9-yl)thio]ethyl}amino)ethyl]sulfonyl)-6-O-methylerythromycin A, 4″-O-(2-[(2-{[6-carboxy-8-dimethylamino-5-oxo-5,8-dihydro-[1,8]naphthyridin-3-yl]thio}ethyl)amino]ethyl}sulfonyl)-6-O-methyl-erythromycin A formate,
4″-O-{2-({3-[6-Carboxy-3,3-dimethyl-7-oxo-1H,7H-[1,3]oxazino[5,4,3-ij]quinolin-9-yl]propyl}amino)ethanesulfonyl}-erythromycin A-(9E)-oxime,
4″-O-{[2-({2-[(2-Carboxy-5-methyl-1-oxo-6,7-dihydro-1H,5H-pyrido[3,2,1-ij]quinolin-9-yl)thio]ethyl}amino)ethyl]sulfonyl}erythromycin A-(9E)-oxime,
4″-O-{2-[3-(3-Carboxy-1,4-dihydro-1-(2-fluoroethyl)-4-oxo-6-quinolinyl)propylamino]ethanesulfonyl}-6-O-methylerythromycin A,
4″-O-{2-({3-[3-Carboxy-1-ethyl-4-oxo-1,4-dihydro-6-quinolinyl]propyl}amino)ethanesulfonyl}-erythromycin A-(9E)-oxime,
4″-O-{2-[3-(3-Carboxy-1,4-dihydro-1-dimethylamino-4-oxo-6-quinolinyl)propylamino]ethanesulfonyl}-azithromycin-11,12-carbonate,
4″-O-{2-[3-(3-Carboxy-1,4-dihydro-1-ethyl-4-oxo-6-quinolinyl) propylamino]ethanesulfonyl}-azithromycin-11,12-carbonate,
4″-O-{2-[3-(3-Carboxy-1,4-dihydro-1-ethyl-4-oxo-6-quinolinyl) propyloxy]ethanesulfonyl}-6-O-methylerythromycin A,
4″-O-{2-({3-[3-Carboxy-1-ethyl-4-oxo-1,4-dihydro-6-quinolinyl]propyl}amino)ethanesulfonyl}-erythromycin A-(9E)-O-methoxymethyloxime,
4″-O-{2-[3-(3-Carboxy-1,4-dihydro-1-Dimethylamino-4-oxo-6-quinolinyl)propylamino]ethanesulfonyl}-erythromycin A-(9E)-oxime-11,12-carbonate,
4″-O-{2-[3-(3-Carboxy-1,4-dihydro-1-ethyl-4-oxo-6-quinolinyl) propylamino]ethanesulfonyl}-erythromycin A-(9E)-oxime-11,12-carbonate,
4″-O-{2-[2-(3-Carboxy-1,4-dihydro-1-ethyl-6-fluoro-4-oxo-[1,8]naphthyridin-7-ylamino)ethylamino]ethanesulfonyl}-erythromycin A-(9E)-oxime-11,12-carbonate,
4″-O-{2-[3-(3-Carboxy-1,4-dihydro-1-(morpholin-4-yl)-4-oxo-6-quinolinyl)propylamino]ethanesulfonyl}-6-O-methylerythromycin A,
4″-O-{2-[3-(3-Carboxy-1,4-dihydro-1-dimethylamino-4-oxo-7-quinolinyl)oxyethylamino]ethanesulfonyl}-6-O-methylerythromycin A,
4″-O-{2-[(3R)-3-({3-[3-carboxy-1-ethyl-4-oxo-1,4-dihydro-6-quinolinyl]propyl}oxy)-1-pyrrolidinyl]ethanesulfonyl}-erythromycin A-(9E)-oxime,
4″-O-{2-[3-(3-Carboxy-1,4-dihydro-1-dimethylamino-4-oxo-6-quinolinyl)propylamino]ethanesulfonyl}-azithromycin diformate salt,
4″-O-{2-[3-(3-Carboxy-1,4-dihydro-1-dimethylamino-4-oxo-6-quinolinyl)propylamino]ethanesulfonyl}-erythromycin A-11,12-carbonate,
4″-O-{2-[3-(3-Carboxy-1,4-dihydro-1-dimethylamino-4-oxo-7-quinolinyl)oxypropylamino]ethanesulfonyl}-6-O-methylerythromycin A,
4″-O-{2-[3-(3-Carboxy-1,4-dihydro-1-dimethylamino-4-oxo-6-quinolinyl)propyloxy]ethanesulfonyl}-6-O-methylerythromycin A,
4″-O-{2-[3-(3-Carboxy-1,4-dihydro-1-amino-4-oxo-7-quinolinyl)propylamino]ethanesulfonyl}-6-O-methylerythromycin A,
4″-O-{[2-({2-[(2-{[2-Carboxy-5-methyl-1-oxo-6,7-dihydro-1H,5H-pyrido[3,2,1-ij]quinolin-9-yl]amino}ethyl)oxy]ethyl}oxy)ethyl]sulfonyl}-6-O-methylerythromycin A,
4″-O-{[2-({2-[(2-{[2-Carboxy-5-methyl-1-oxo-6,7-dihydro-1H,5H-pyrido[3,2,1-ij]quinolin-9-yl]amino}ethyl)oxy]ethyl}amino)ethyl]sulfonyl}-6-O-methylerythromycin A,
4″-O-[(2-{[3-(6-Carboxy-3,3-dimethyl-7-oxo-2,3-dihydro-7H-[1,4]oxazino[2,3,4-ij]quinolin-9-yl)propyl]amino}ethyl)sulfonyl]-6-O-methyl erythromycin A,
4″-O-{2-[3-(3-Carboxy-1,4-dihydro-1-ethyl-4-oxo-6-quinolinyl)propylamino]ethanesulfonyl}-erythromycin A-11,12-carbonate,
4″-O-{2-[2-(3-Carboxy-1,4-dihydro-1-ethyl-6-fluoro-4-oxo-[1,8]naphthyridin-7-ylamino)ethylamino]ethanesulfonyl}-erythromycin A-11,12-carbonate,
4″-O-{2-[3-(3-Carboxy-1,4-dihydro-1-ethyl-4-oxo-6-quinolinyl)propylamino]ethanesulfonyl}-9-(S)-dihydroerythromycin A-9,11-ethylidene acetal,
4″-O-{2-[3-(6-Carboxy-7-oxo-1H,7H-[1,3]oxazino[5,4,3-ij]quinolin-9-yl)propylamino]ethanesulfonyl}-erythromycin A-(9E)-O-methoxymethyloxime,
4″-O-{2-[3-(3-Carboxy-1,4-dihydro-1-methyl-4-oxo-6-quinolinyl) propylamino]ethanesulfonyl}-6-O-methylerythromycin A,
4″-O-{2-[3-(3-Carboxy-1,4-dihydro-1-methyl-4-oxo-6-quinolinyl) propylamino]ethanesulfonyl}-erythromycin A-(9E)-oxime-11,12-carbonate,
4″-O-{2-[3-(3-Carboxy-1,4-dihydro-1-ethenyl-4-oxo-6-quinolinyl) propylamino]ethanesulfonyl}-6-O-methylerythromycin A,
4″-O-{2-{[2-({2-[(3-Carboxy-7-chloro-1-cyclopropyl-4-oxo-1,4-dihydro-6-quinolinyl)amino]ethyl}oxy)ethyl]oxy}ethanesulfonyl}-6-O-methylerythromycin A,
4″-O-{2-{[2-({2-[(3-Carboxy-7-chloro-1-cyclopropyl-4-oxo-1,4-dihydro-6-quinolinyl)amino]ethyl}oxy)ethyl]oxy}ethanesulfonyl}-azithromycin,
4″-O-{2-(2-[(2-{[2-(3-Carboxy-1-ethyl-4-oxo-1,4-dihydro-6-quinolinyl)ethyl]oxy}ethyl)oxy]ethylamino)ethanesulfonyl}-erythromycin A-(9E)-O-methoxymethyloxime,
4″-O-{2-(2-[(2-{[2-(3-Carboxy-1-ethyl-4-oxo-1,4-dihydro-6-quinolinyl)ethyl]oxy}ethyl)oxy]ethylamino)ethanesulfonyl}-6-O-methylerythromycin A,
4″-O-{2-[3-(3-Carboxy-1,4-dihydro-1-methoxy-4-oxo-6-quinolinyl) propylamino]ethanesulfonyl}-6-O-methylerythromycin A,
4″-O-{2-({3-[3-Carboxy-1-methoxy-4-oxo-1,4-dihydro-6-quinolinyl]propyl}amino)ethanesulfonyl}-erythromycin A-(9E)-O-methoxymethyloxime,
4″-O-{2-[2-(3-Carboxy-1,4-dihydro-1-ethyl-4-oxo-7-quinolinyloxy)ethylamino]ethanesulfonyl}-6-O-methylerythromycin A formate,
4″-O-{2-[2-(3-Carboxy-1,4-dihydro-1-ethyl-4-oxo-7-quinolinyloxy)ethylamino]ethanesulfonyl}erythromycin A-(9E)-oxime formate,
4″-O-{2-[2-(3-Carboxy-1,4-dihydro-1-ethyl-4-oxo-7-quinolinyloxy)ethylamino]ethanesulfonyl}erythromycin A-11,12-carbonate formate,
4″-O-{[2-({2-[(2-Carboxy-5-methyl-1-oxo-6,7-dihydro-1H,5H-pyrido[3,2,1-ij]quinolin-9-yl)amino]ethyl}amino)ethyl]sulfonyl}-6-O-methylerythromycin A,
4″-O-{2-[{2-(3-Carboxy-1,4-dihydro-1-ethyl-6-fluoro-4-oxo-[1,8]naphthyridin-7-ylamino)ethyl}amino]ethanesulfonyl-erythromycin A-(9E)-oxime,
4″-O-{2-[{2-(3-Carboxy-1,4-dihydro-1-ethyl-4-oxo-[1,8]naphthyridin-7-ylamino)ethyl)amino]ethanesulfonyl}-}-6-O-methylerythromycin A,
4″-O-{2-[3-(3-Carboxy-1,4-dihydro-1-ethyl-4-oxo-6-quinolinyl) propylamino]ethanesulfonyl}-erythromycin A (9E)-O-methyloxime,
4″-O-{2-[2-(3-Carboxy-1-ethyl-6-fluoro-4-oxo-1,4-dihydro-[1,8]naphthyridin-7-ylamino)ethyl)amino]ethanesulfonyl}-erythromycin A (9E)-O-methyloxime,
4″-O-{2-{2-({3-(3-Carboxy-1-ethyl-4-oxo-1,4-dihydro-6-quinolinyl)]propyl}oxy)ethylamino}ethanesulfonyl}-azithromycin triformate,
4″-O-{2-{[2-({3-[3-Carboxy-1-cyclopropyl-4-oxo-1,4-dihydro-6-quinolinyl]propyl}oxy)ethyl]oxy}ethanesulfonyl}-azithromycin,
4″-O-{2-{[2-({3-[3-Carboxy-1-ethyl-4-oxo-1,4-dihydro-6-quinolinyl]propyl}oxy)ethyl]oxy}ethanesulfonyl}-azithromycin,
4″-O-{2-{[2-({2-[(3-Carboxy-1-cyclopropyl-4-oxo-1,4-dihydro-6-quinolinyl)amino]ethyl}oxy)ethyl]oxy}ethanesulfonyl}-azithromycin,
4″-O-{2-{[2-({2-[(3-Carboxy-1-cyclopropyl-4-oxo-1,4-dihydro-6-quinolinyl)methylamino]ethyl}oxy)ethyl]oxy}ethanesulfonyl}-azithromycin,
4″-O-{2-[2-(3-Carboxy-1,4-dihydro-1-ethyl-6-fluoro-4-oxo-[1,8]naphthyridin-7-ylamino)ethylamino]ethanesulfonyl}-erythromycin A-(9E)-O-cyanomethyloxime,
4″-O-{2-[3-(3-Carboxy-1,4-dihydro-1-dimethylamino-4-oxo-6-quinolinyl)propylamino]ethanesulfonyl}-erythromycin A-(9E)-O-(5-methylisoxazol-3-yl)-methyloxime,
4″-O-{2-[3-(3-Carboxy-1,4-dihydro-1-ethyl-4-oxo-6-quinolinyl)propylamino]ethanesulfonyl}-erythromycin A-(9E)-O—(N,N-dimethylacetamido)-oxime,
4″-O-{2-({3-[3-carboxy-1-ethyl-4-oxo-1,4-dihydro-6-quinolinyl]propyl}amino)ethanesulfonyl}-erythromycin A (9E)-O-2-(diethylamino)ethyloxime,
4″-O-{2-[{2-(3-Carboxy-1,4-dihydro-1-ethyl-6-fluoro-4-oxo-[1,8]naphthyridin-7-ylamino)ethyl}amino]ethanesulfonyl}-erythromycin A (9E)-O-2-(diethylamino)ethyloxime,
4″-O-{2-[3-(3-Carboxy-1,4-dihydro-1-Dimethylamino-4-oxo-6-quinolinyl)propylamino]ethanesulfonyl}-erythromycin A (9E)-2-(diethylamino)ethyloxime,
4″-O-[(2-{[3-(6-Carboxy-7-oxo-1H,7H-[1,3]oxazino[5,4,3-ij]quinolin-9-yl)propyl]amino}ethanesulfonyl]-erythromycin A (9E)-oxime,
4″-O-[(2-{[3-(6-carboxy-7-oxo-1H,7H-[1,3]oxazino[5,4,3-ij]quinolin-9-yl)propyl]amino}ethanesulfonyl]-erythromycin A (9E)-O-cyanomethyloxime,
4″-O-{2-[3-(7-Carboxy-8-oxo-3,4-dihydro-1H,8H-[1,4]oxazepino[6,5,4-ij]quinoline-10-yl)propylamino]ethanesulfonyl}-6-O-methyl-erythromycin A
4″-O-{2-({3-[3-Carboxy-1-ethyl-4-oxo-1,4-dihydro-6-quinolinyl]propyl}methylamino)ethanesulfonyl}-erythromycin A-(9E)-oxime formate salt,
and pharmaceutically acceptable derivatives thereof.

9. A process for the preparation of a compound as claimed in claim 1, or a pharmaceutically acceptable derivative thereof, which comprises reacting a compound of formula (II), wherein R² is optionally a hydroxyl protecting group 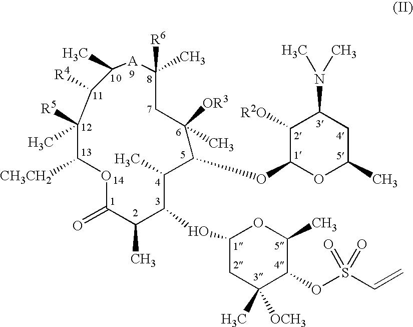
with a compound of formula HU^{1z}R^{13z} (III) or an amine (IV), (IVA) or (IVB) 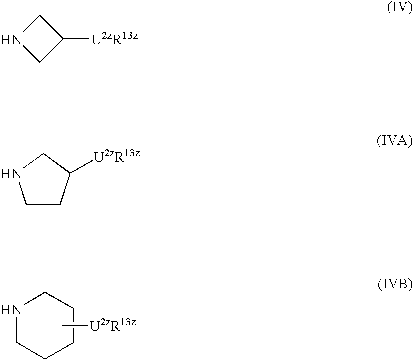
and thereafter, if required, subjecting the resulting compound to one or more of the following operations:
i) removal of the protecting group R²,
ii) conversion of U^{1z}R^{13z} or U^{2z}R^{3z} to U¹R¹³ or U²R¹³, and
iii) conversion of the resultant compound of formula (I) into a pharmaceutically acceptable derivative thereof.

10. A compound as claimed in claim 1, or a pharmaceutically acceptable derivative thereof, for use in therapy.

11. A compound as claimed in claim 1, or a pharmaceutically acceptable derivative thereof, for use in the treatment or prophylaxis of systemic or topical microbial infections in a human or animal body.

12. (canceled)

13. A method for the treatment of the human or non-human animal body to combat microbial infection comprising administration to a body in need of such treatment of an effective amount of a compound as claimed in claim 1, or a pharmaceutically acceptable derivative thereof.

14. A pharmaceutical composition comprising a compound as claimed in claim 1, or a pharmaceutically acceptable derivative thereof, in association with a pharmaceutically acceptable excipient, diluent and/or carrier.
